(19) United States
(12) Patent Application Publication
(10) Pub. No.: US 2025/0228857 A1
(43) Pub. Date: Jul. 17, 2025

(54) BIOSENSOR AND USES THEREOF

(71) Applicant: The Johns Hopkins University, Baltimore, MD (US)

(72) Inventors: Oscar Eduardo Reyes Gaido, Baltimore, MD (US); Mark Anderson, Baltimore, MD (US); Elizabeth Luczak, Baltimore, MD (US)

(21) Appl. No.: 18/850,935

(22) PCT Filed: Mar. 24, 2023
(86) PCT No.: PCT/US2023/016315

§ 371 Date: Sep. 25, 2024

Related U.S. Application Data
(60) Provisional application No. 63/323,968, filed on Mar. 25, 2022.

(51) Int. Cl. A61K 31/519; A61K 31/4709; A61K 31/4745; A61K 31/506; C12Q 1/48

(52) U.S. Cl. CPC A61K 31/519; A61K 31/4709; A61K 31/4745; A61K 31/506; C12Q 1/485; C12Y 207/11017; G01N 2333/912; G01N 2500/10

(57) ABSTRACT

Compositions include biosensors and enzyme substrates for use in the biosensors. Methods of identifying modulators of enzymatic activity include the biosensors.

The present application claims the benefit of priority of U.S. provisional application No. 63/323,968 filed Mar. 25, 2022, which is incorporated by reference herein in its entirety.

### STATEMENT REGARDING FEDERALLY SPONSORED RESEARCH

This invention was made with government support under grant HL140034 awarded by the National Institutes of Health. The government has certain rights in the invention.

### BACKGROUND

Ca²⁺/Calmodulin-dependent protein kinase II (CaMKII) is a highly validated cause or contributor to major cardiac illnesses, including heart failure, myocardial infarction, and arrhythmias. Excessive CaMKII activity causes intracellular Ca²⁺ dysregulation, inflammation, maladaptive transcription, and cell death. Yet, there are currently no approved CaMKII inhibiting therapies. Thus, development of safe and effective CaMKII inhibitors is a translational priority.

### SUMMARY

We now provide a novel biosensor comprising: an enzyme substrate, a detectably labelled protein, and a phospho-amino acid binding protein. In certain embodiments, the enzyme substrate is a kinase. In certain embodiments, the kinase is calcium/calmodulin-dependent protein kinase II (CaMKII). In certain embodiments, the CaMKII substrate comprises an amino acid sequence having at least about 70% sequence identity to MHRQETVDCLK (SEQ ID NO: 1) or to X_{N+1}MHRQETVDCLK Y_{N+1} (SEQ ID NO: 2) wherein X or Y comprise amino acids analogs or variants thereof and N=0, 1, 2, 3, 4 or more. In certain embodiments, the CaMKII substrate comprises an amino acid sequence having at least about 90% sequence identity to MHRQETVDCLK (SEQ ID NO: 1) or SEQ ID NO: 2. In certain embodiments, the CaMKII substrate comprises an amino acid sequence having at least about 95% sequence identity to MHRQETVDCLK (SEQ ID NO: 1) or SEQ ID NO: 2. In certain embodiments, the CaMKII substrate comprises the amino acid sequence MHRQETVDCLK (SEQ ID NO: 1) or SEQ ID NO: 2. In certain embodiments, the detectably labelled protein comprises a radio labeled molecule, a fluorophore, a radiochemical, a luminescent compound, an electron-dense reagent, an enzyme, biotin, a radioactive compound, a non-radioactive compound, digoxigenin or a hapten. In certain embodiments, the detectably labelled protein comprises a fluorophore. In certain embodiments, the biosensor further comprises an intracellular or extracellular localization sequence. In certain embodiments, the localization sequence is fused to the biosensor. In certain embodiments, the localization sequence is covalently linked through a flexible linker. In certain embodiments, the flexible linker is a polypeptide comprising at least five amino acids.

In certain embodiments, an expression vector encoding a biosensor comprising an enzyme substrate, a detectably labelled protein, and a phospho-amino acid binding protein. In certain embodiments, the enzyme substrate is a kinase. In certain embodiments, the kinase is calcium/calmodulin-dependent protein kinase II (CaMKII). In certain embodiments, the CaMKII substrate comprises an amino acid sequence having at least about 70% sequence identity to MHRQETVDCLK (SEQ ID NO: 1) or SEQ ID NO: 2. In certain embodiments, the CaMKII substrate comprises an amino acid sequence having at least about 90% sequence identity to MHRQETVDCLK (SEQ ID NO: 1) or SEQ ID NO: 2. In certain embodiments, the CaMKII substrate comprises an amino acid sequence having at least about 95% sequence identity to MHRQETVDCLK (SEQ ID NO. 1) or SEQ ID NO: 2. In certain embodiments, the CaMKII substrate comprises the amino acid sequence MHRQETVDCLK (SEQ ID NO: 1) or SEQ ID NO: 2.

In certain embodiments, a method of identifying modulators of kinases comprises contacting a biosensor or a cell expressing a biosensor, with one or more candidate agents, wherein the biosensor comprises an enzyme substrate, a detectably labelled protein, and a phospho-amino acid binding protein; assaying for the presence or absence of a signal from the detectable label; and, identifying modulators of kinases. In certain embodiments, a signal from the detectable label is identified as an activator of the kinase. In certain embodiments, the absence of a signal from the detectable label is identified as an inhibitor of the kinase. In certain embodiments, the kinase is calcium/calmodulin-dependent protein kinase II (CaMKII). In certain embodiments, the CaMKII comprises an amino acid sequence having at least about 70% sequence identity to MHRQETVDCLK (SEQ ID NO: 1) or SEQ ID NO: 2. In certain embodiments, the CaMKII comprises an amino acid sequence having at least about 90% sequence identity to MHRQETVDCLK (SEQ ID NO: 1) or SEQ ID NO: 2. In certain embodiments, the CaMKII comprises an amino acid sequence having at least about 95% sequence identity to MHRQETVDCLK (SEQ ID NO: 1) or SEQ ID NO: 2. In certain embodiments, the CaMKII comprises the amino acid sequence MHRQETVDCLK (SEQ ID NO: 1) or SEQ ID NO: 2. In certain embodiments, the detectably labelled protein comprises a radio labeled molecule, a fluorophore, a radiochemical, a luminescent compound, an electron-dense reagent, an enzyme, biotin, a radioactive compound, a non-radioactive compound, digoxigenin or a hapten. In certain embodiments, the detectably labelled protein comprises a fluorophore.

In certain embodiments, a calcium/calmodulin-dependent protein kinase II (CaMKII) substrate comprising an amino acid sequence having at least about 70% sequence identity to MHRQETVDCLK (SEQ ID NO: 1). In certain embodiments, the CaMKII substrate comprises an amino acid sequence having at least about 90% sequence identity to MHRQETVDCLK (SEQ ID NO: 1). In certain embodiments, the CaMKII substrate comprises an amino acid sequence having at least about 95% sequence identity to MHRQETVDCLK (SEQ ID NO: 1). In certain embodiments, the CaMKII substrate comprises the amino acid sequence MHRQETVDCLK (SEQ ID NO: 1). In certain embodiments, CaMKII substrate further comprises a localization sequence. In certain embodiments, the localization sequence is an intracellular or extracellular localization sequence. In certain embodiments, the extracellular localization sequence comprises a ligand or receptor sequence. In certain embodiments, the localization sequence is covalently linked through a flexible linker. In certain embodiments, the flexible linker is a polypeptide comprising at least five amino acids.

In certain embodiments, a calcium/calmodulin-dependent protein kinase II (CaMKII) substrate comprising an amino acid sequence having at least about 70% sequence identity to X_{N+1}MHRQETVDCLK Y_{N+1} (SEQ ID NO: 2) wherein X or Y comprise amino acids analogs or variants thereof and N=0, 1, 2, 3, 4 or more. In certain embodiments, the CaMKII substrate comprises an amino acid sequence having at least about 90% sequence identity to SEQ ID NO: 2. In certain embodiments, the CaMKII substrate comprises an amino acid sequence having at least about 95% sequence identity to SEQ ID NO: 2. In certain embodiments, the CaMKII substrate comprises the amino acid sequence SEQ ID NO: 2. In certain embodiments, the CaMKII substrate further comprises a localization sequence. In certain embodiments, the localization sequence is an intracellular or extracellular localization sequence. In certain embodiments, the extracellular localization sequence comprises a ligand or receptor sequence. In certain embodiments, the localization sequence is covalently linked through a flexible linker. In certain embodiments, the flexible linker is a polypeptide comprising at least five amino acids.

In certain embodiments, a host cell comprises an expression vector encoding SEQ ID NOS: 1 or 2. In certain embodiments, the host cell comprises SEQ ID NOS: 1 or 2.

In certain embodiments, a kit comprises a synthetic biosensor, an expression vector encoding SEQ ID NOS: 1 or 2, the host cell comprising the vector or SEQ ID NOS: 1 or 2.

In certain embodiments, a method of preventing and treating a heart disease comprises administering to a subject in need thereof, a pharmaceutical composition comprising a therapeutically effective amount of ruxolitinib, crenolanib, baricitinib, abemaciclib, silmitasertib, or combinations thereof. In certain embodiments, the pharmaceutical composition comprises a therapeutically effective amount of ruxolitinib. In certain embodiments, the heart disease comprises catecholaminergic polymorphic ventricular tachycardia (CPVT), atrial fibrillation, arrhythmias, heart failure, cardiomyopathy, heart valve disease, pericardial disease, peripheral vascular disease, rheumatic heart disease, post-operative atrial fibrillation, post-myocardial infarction ventricular tachycardia, electrical storm, or combinations thereof. In certain embodiments, the heart disease is an arrhythmia.

In certain embodiments, a method of treating a subject comprises administering to a subject in need thereof, a pharmaceutical composition comprising a therapeutically effective amount of ruxolitinib. In certain embodiments, the subject in need thereof is diagnosed as suffering from Timothy Syndrome, Duchenne's muscular dystrophy, Ankyrin B mutations, glycoside toxicity, heart failure, or alcoholic cardiomyopathy. In certain embodiments, the subject is identified as having an RyR2 mutation.

### Definitions

Unless specifically defined otherwise, all technical and scientific terms used herein shall be taken to have the same meaning as commonly understood by one of ordinary skill in the art (e.g., in cell culture, molecular genetics, and biochemistry).

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the term “about” in the context of a numerical value or range means 10% of the numerical value or range recited or claimed, unless the context requires a more limited range.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

As used herein, the term “agent” or “candidate agent” is meant to encompass any molecule, chemical entity, composition, drug, therapeutic agent, chemotherapeutic agent, or biological agent capable of preventing, ameliorating, or treating a dysfunction or other medical condition. The term includes small molecule compounds, antisense oligonucleotides, siRNA reagents, antibodies, antibody fragments bearing epitope recognition sites, such as Fab, Fab′, F(ab′)₂ fragments, Fv fragments, single chain antibodies, antibody mimetics (such as DARPins, affibody molecules, affilins, affitins, anticalins, avimers, fynomers, Kunitz domain peptides and monobodies), peptoids, aptamers; enzymes, peptides organic or inorganic molecules, natural or synthetic compounds and the like. An agent can be assayed in accordance with the methods of the disclosure at any stage during clinical trials, during pre-trial testing, or following FDA-approval.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

As used in this specification and the appended claims, the term “or” is generally employed in its sense including “and/or” unless the content clearly dictates otherwise.

A “derivative” polypeptide or peptide is one that is modified, for example, by glycosylation, pegylation, phosphorylation, sulfation, reduction/alkylation, acylation, chemical coupling, or mild formalin treatment. A derivative may also be modified to contain a detectable label, either directly or indirectly, including, but not limited to, a radioisotope, fluorescent, and enzyme label.

The term “identical” or percent “identity,” in the context of two or more nucleic acids or polypeptide sequences, refer to two or more sequences or subsequences that are the same or have a specified percentage of amino acid residues or nucleotides that are the same (e.g., 50%, 55%, 60%, 65%, 70%, 75%, 80%, 85%, 90%, 91%, 92%, 93%, 94%, 95%, 96%, 97%, 98%, 99%, or more identity over a specified region, e.g., of an entire polypeptide sequence or an individual domain thereof), when compared and aligned for maximum correspondence over a comparison window, or designated region as measured using a sequence comparison algorithm or by manual alignment and visual inspection. Such sequences that are at least about 80% identical are said to be “substantially identical.” In some embodiments, two sequences are 100% identical. In certain embodiments, two sequences are 100% identical over the entire length of one of the sequences (e.g., the shorter of the two sequences where the sequences have different lengths). In various embodiments, identity may refer to the complement of a test sequence. In some embodiments, the identity exists over a region that is at least about 10 to about 100, about 20 to about 75, about 30 to about 50 amino acids or nucleotides in length. In certain embodiments, the identity exists over a region that is at least about 50 amino acids in length, or more preferably over a region that is 100 to 500, 100 to 200, 150 to 200, 175 to 200, 175 to 225, 175 to 250, 200 to 225, 200 to 250 or more amino acids in length.

For sequence comparison, typically one sequence acts as a reference sequence, to which test sequences are compared. In various embodiments, when using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window” refers to a segment of any one of the number of contiguous positions (e.g., least about 10 to about 100, about 20 to about 75, about 30 to about 50, 100 to 500, 100 to 200, 150 to 200, 175 to 200, 175 to 225, 175 to 250, 200 to 225, 200 to 250) in which a sequence may be compared to a reference sequence of the same number of contiguous positions after the two sequences are optimally aligned. In various embodiments, a comparison window is the entire length of one or both of two aligned sequences. In some embodiments, two sequences being compared comprise different lengths, and the comparison window is the entire length of the longer or the shorter of the two sequences. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

In various embodiments, an algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 may be used, with the parameters described herein, to determine percent sequence identity for nucleic acids and proteins. Software for performing BLAST analyses is publicly available through the National Center for Biotechnology Information, as known in the art. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff& Henikoff, Proc. Natl. Acad. Sci. USA 89:10915 (1989)) alignments (B) of 50, expectation (E) of 10, M=5, N=−4, and a comparison of both strands.

A “label” or a “detectable label” is a composition detectable by spectroscopic, photochemical, biochemical, immunochemical, or chemical means. For example, useful labels include radio labeled molecules fluorophores, radiochemical, luminescent compounds, electron-dense reagents, enzymes (e.g., as commonly used in an ELISA), biotin, radioactive compounds, non-radioactive compounds, digoxigenin, or haptens and proteins which can be made detectable, e.g., by incorporating a label into the peptide.

The term “ligand,” includes any compound, composition or molecule capable of specifically or substantially specifically (that is with limited cross-reactivity) binding another compound or molecule, which, in the case of immune-recognition contains an epitope. In many instances, the ligands are antibodies, such as polyclonal or monoclonal antibodies. “Ligands” also include derivatives or analogs of antibodies, including without limitation: Fv fragments; single chain Fv (scFv) fragments; Fab′ fragments; F(ab′)₂ fragments; humanized antibodies and antibody fragments; camelized antibodies and antibody fragments; and multivalent versions of the foregoing. Multivalent binding reagents also may be used, as appropriate, including without limitation: monospecific or bispecific antibodies, such as disulfide stabilized Fv fragments, scFv tandems ((scFv)fragments), diabodies, tribodies or tetrabodies, which typically are covalently linked or otherwise stabilized (i.e., leucine zipper or helix stabilized) scFv fragments. “Ligands” also include peptoids, peptide or nucleic acid aptamers, or antibody mimetics such as DARPins, affibody molecules, affilins, affitins, anticalins, avimers, fynomers, Kunitz domain peptides and monobodies.

As used herein, “modulate,” “modulates” or “modulation” refers to enhancement (e.g., an increase) or inhibition (e.g., diminished, reduced or suppressed) of the specified activity or expression of a gene, polynucleotides, oligonucleotides, proteins, polypeptides, peptides or combinations thereof. Accordingly, a “modulator” enhances or inhibits expression, function or activity of gene, polynucleotides, oligonucleotides, etc.

The term “enhancement,” “enhance,” “enhances,” or “enhancing” refers to an increase in the specified parameter (e.g., at least about a 1.1-fold, 1.25-fold, 1.5-fold, 2-fold, 3-fold, 4-fold, 5-fold, 6-fold, 8-fold, 10-fold, twelve-fold, or even fifteen-fold or more increase) and/or an increase in the specified activity of at least about 5%, 10%, 25%, 35%, 40%, 50%, 60%, 75%, 80%, 90%, 95%, 97%, 98%, 99% or 100%.

The term “inhibit,” “diminish,” “reduce” or “suppress” refers to a decrease in the specified parameter (e.g., at least about a 1.1-fold, 1.25-fold, 1.5-fold, 2-fold, 3-fold, 4-fold, 5-fold, 6-fold, 8-fold, 10-fold, twelve-fold, or even fifteen-fold or more increase) and/or a decrease or reduction in the specified activity of at least about 5%, 10%, 25%, 35%, 40%, 50%, 60%, 75%, 80%, 90%, 95%, 97%, 98%, 99% or 100%. These terms are intended to be relative to a reference or control.

The term “fluorophore” includes any compound, composition or molecule capable of emitting light in response to irradiation. In many instances, fluorophores emit light in the visible region of light. In other instances, the fluorophores can emit light in the non-visible regions of light, such as ultraviolet, near-ultraviolet, near-infrared, and infrared. For example and without limitation, examples of fluorophores include: quantum dots; nanoparticles; fluorescent proteins, such as green fluorescent protein and yellow fluorescent protein; heme-based proteins or derivatives thereof, carbocyanine-based chromophores, such as IRDye 800CW, Cy 3, and Cy 5; coumarin-based chromophores, such as (7-diethylamino-3-(4′-maleimidylphenyl)-4-methylcoumarin) (CPM); fluorine-based chromophores, such as fluorescein, fluorescein isothiocyanate (FITC); and numerous ALEXA FLUOR™ chromophores and ALEXA FLUOR™ bioconjugates, which absorb in the visible and near-infrared spectra. The emission from the fluorophores can be detected by any number of methods, including but not limited to, fluorescence spectroscopy, fluorescence microscopy, fluorimeters, fluorescent plate readers, infrared scanner analysis, laser scanning confocal microscopy, automated confocal nanoscanning, laser spectrophotometers, fluorescent-activated cell sorters (FACS), image-based analyzers and fluorescent scanners (e.g., gel/membrane scanners).

As used herein, the term “chromophore” refers to a substituent which, with another chromophore, can be used for energy transfer (e.g., FRET assay).

The term “chemiluminescent compound” includes any compound, composition or molecule capable of emitting light in response to a chemical reaction. A “bioluminescent compound” refers to a naturally occurring form of a chemiluminescent compound. Examples of chemiluminescent compounds include: lucigenin, luminol. Examples of bioluminescent compounds include: luciferins, coelenterazines. The emission from chemiluminescent compounds can be detected by luminometers or scanning spectrometers.

The term “luminescent component” or “luminescent compound” as used herein refers to a component capable of absorbing energy, such as electrical (e.g., electro-luminescence), chemical (e.g., chemi-luminescence) or acoustic energy and then emitting at least some fraction of that energy as light over time. The term “component” as used herein includes discrete compounds, molecules, bioluminescent proteins and macro-molecular complexes or mixtures of luminescent and non-luminescent compounds or molecules that act to cause the emission of light.

The term “radiochemical” is intended to encompass any organic, inorganic or organometallic compound comprising a covalently-attached radioactive isotope, any inorganic radioactive ionic solution (e.g., Na[¹⁸F]F ionic solution), or any radioactive gas (e.g., [¹¹C]CO₂), particularly including radioactive molecular imaging probes intended for administration to a patient (e.g., by inhalation, ingestion, or intravenous injection) for tissue imaging purposes, which are also referred to in the art as radiopharmaceuticals, radiotracers, or radioligands. The compounds could also be readily adapted for synthesis of any radioactive compound comprising a radionuclide, including radiochemicals useful in other imaging systems, such as single photon emission computed tomography (SPECT).

The term “sample” as used herein refers to a biological sample obtained for the purpose of evaluation in vitro. With regard to the methods disclosed herein, the sample or patient sample preferably may comprise any body fluid or tissue. In some embodiments, the bodily fluid includes, but is not limited to, blood, plasma, serum, lymph, breast milk, saliva, mucous, semen, vaginal secretions, cellular extracts, inflammatory fluids, cerebrospinal fluid, feces, vitreous humor, or urine obtained from the subject. In some aspects, the sample is a composite panel of at least two of a blood sample, a plasma sample, a serum sample, and a urine sample. In exemplary aspects, the sample comprises blood or a fraction thereof (e.g., plasma, serum, fraction obtained via leukopheresis). Preferred samples are whole blood, serum, plasma, or urine. A sample can also be a partially purified fraction of a tissue or bodily fluid.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### BRIEF DESCRIPTION OF THE DRAWINGS

FIGS. 1A-1I demonstrate that CaMKAR is a sensitive and specific CaMKII activity biosensor. (FIG. 1A) Schematic depiction of CaMKII Activity Reporter (CaMKAR)—(cpGFP: circularly permutated green fluorescent protein; PAABD: phosphorylated amino acid binding domain). The phospho-substrate threonine shown in red. (FIG. 1B) Fluorescence confocal microscopy of CaMKAR-expressing 293T cells treated with ionomycin (5 μM). R=ratio of 488 nm-excited and 405 nm-excited intensities. Scale bar=20 μm. Third row is pseudo-colored to display fold-change over baseline. (FIG. 1C) Summary data for CaMKAR signal in HEK293T cells treated with vehicle (n=2,488-2,615 cells), ionomycin (5 μM; n=1,733-2,220 cells); pre-treatment with the CaMKII inhibitor (inh) AS100397 (10 μM; n=1,305-1,348 cells) or post-treatment with EGTA (5 mM; n=1,474-1,746). No exogenous CaMKII added. R/R0=R normalized to mean baseline. (FIG. 1D) CaMKAR signal in HEK293T cells expressing doxycycline-induced wild type, kinase dead (KD; K43M mutation), or constitutively active (CA; T287D mutation) CaMKIIδC. N=3 wells per condition. (FIG. 1E) DeadCaMKAR(T6A mutation)-expressing HEK293T cells infected with wild-type (WT; n=mean of 3 wells), constitutively active (CA, n=mean of 3 wells), kinase dead (KD; n=mean of 3 wells) CaMKIIδC or uninfected (n=mean of 3 wells) and then stimulated with ionomycin. (FIG. 1F) CaMKAR-expressing HEK293T cells co-transfected with constitutively active (CA) CaMKI (n=1,471 cells), CaMKIV (n=1,732 cells), or CaMKIIδ (n=1,816 cells). (FIG. 1G) CaMKAR-expressing 293T cells treated with Fsk (50 μM)/IBMX (100 μM; n=1,834-1891 cells) or ionomycin (n=1,997-2,293). (FIG. 1H) CaMKAR-expressing 293T cells treated with PMA (100 ng/mL; n=696-737 cells), ionomycin (5 μM; n=767-916), or vehicle. (FIG. 1I) CaMKAR-expressing 293T cells treated with PDBu (200 nM; n=1706-1736 cells) or ionomycin (5 μM; n=1206-1228). Data shown as mean±SEM (unless too small to display). Arrows denote treatment start. All observations taken from >3 biological replicates. ns=p>0.05, ****p<0.0001; significance was determined via two-way ANOVA and Sidak's multiple comparisons test (FIGS. 1D, 1G-II); linear regression (FIG. 1E); one-way ANOVA with Dunnett's multiple comparisons test (FIG. 1F).

FIGS. 2A-2E demonstrate that CaMKAR-based screen identifies CaMKII inhibitors amongst drugs in clinical use. (FIG. 2A) A schematic depiction of CaMKAR-based high throughput screen. K562 cells were co-infected with CaMKAR- and constitutively active CaMKIIδT287D-encoding lentiviruses. K562CaMKII-CaMKAR cells were screened against the Johns Hopkins Drug Library v3.0. (FIG. 2B) Drugs ranked according to in cellulo CaMKII inhibition in primary screen. CaMKII inhibition % defined by min-max normalization using means of control groups (right; same data as FIG. 11B). 118 selected hits shown in blue. Positive control staurosporine shown in magenta. Dashed line represents hit selection threshold (see Methods). Data shown are subset from complete data set). (FIG. 2C) Hits from FIG. 2B ranked according to in vitro CaMKII inhibition as detected by CaMKAR secondary screen. CaMKII inhibition normalized against untreated control. 13 identified hits in blue. Staurosporine shown in magenta. Data shown are subset from complete data set. (FIG. 2D) Five CaMKII inhibitory drugs and their intended on-targets in the human kinase homology dendrogram. Kinase families: AGC=Containing PKA, PKG, PKC families, CAMK=Calcium/calmodulin-dependent protein kinase, CKI=Casein Kinase 1, CMGC=Containing CDK, MAPK, GSK3, CLK, STE=Homologs of yeast Sterile 7, Sterile 11, Sterile 20, TK=Tyrosine kinase, TKL=Tyrosine kinaselike. (FIG. 2E) IC50 (left) and cell viability (right) curves from 293T cells expressing CaMKAR and CaMKIIT287D and exposed to drugs in FIG. 2E and tool inhibitor (AS100397, 10 mM). Measurements in FIG. 2E done in biological triplicate, complete data set shown in FIGS. 12A, 12B.

FIGS. 3A-3H show which drugs in clinical use inhibit CaMKII in cardiac cells. (FIG. 3A) Fluorescence imaging timelapse of CaMKAR expressing neonatal rat ventricular cardiomyocytes (NRVMs) and (FIG. 3B) summary data during 3 Hz field pacing (n=1,065-1,160 cells). R/R0=R normalized to mean R prior to stimulation. Scale bar=50 μm. (FIG. 3C) Effect of treatment with vehicle (n=317 cells), AS100397 (n=151), ruxolitinib (n=148), crenolanib (n=156), abemaciclib (n=138), baricitinib (n=136), and silmitasertib (n=149) on pacing-induced CaMKII activity in NRVMs. CaMKII inhibition % defined by min-max normalization between untreated and maximally stimulated CaMKAR signal. (FIG. 3D) CaMKII inhibition in NRVMs treated with drugs adjusted to their maximum human plasma concentration during 3 Hz pacing: ruxolitinib (1.51 μM, n=384 cells), crenolanib (478 nM, n=302), abemaciclib (243 nM, n=302), baricitinib (58 nM n=334), and silmitasertib (3.42 μM, n=285). (FIG. 3E) Summary data from D at 60 seconds post stimulation. (FIG. 3F) CaMKAR timelapse imaging in NRVMs treated with ruxolitinib after initiation of pacing and CaMKII activation. Black arrow denotes pacing start, blue arrow denotes addition of ruxolitinib (n=3 wells) or control vehicle (n=3 wells). (FIG. 3G) IC₅₀ curves for ruxolitinib (n=308-384 per data point) and DiOHF (n=305-363) in NRVMs against pacing-induced CaMKII activity. (FIG. 3H) Cell viability after 48 hour compound incubation. All measurements are taken from >3 biological replicates. ns=p>0.05, ****p<0.0001; significance determined via one-way ANOVA and Dunnett's multiple comparisons test (FIGS. 3C,3 E); two-way ANOVA and Tukey's multiple comparisons test (FIG. 3F).

FIGS. 4A-4C demonstrate that ruxolitinib inhibits CaMKII in vivo. (FIG. 4A) immunoblot and (FIGS. 4B to 4C) quantitation of phospholamban (PLN) and threonine-17 phosphorylated phospholamban in whole heart lysates from mice treated with intraperitoneal ruxolitinib (Rux) for 10 minutes prior to isoproterenol (ISO) stimulation. P=pentameric PLN; M=monomeric PLN. Data points represent individual mice. ****p<0.0001; significance determined via one-way ANOVA and Tukey's

FIGS. 5A-5F demonstrate that ruxolitinib suppresses abnormal calcium signaling in a cellular model of CPVT. Induced pluripotent stem cells (iPSCs) with the dominant RyR2 mutation (p.S404R) were obtained from a patient with a clinical diagnosis of catecholaminergic polymorphic ventricular tachycardia (CPVT) and differentiated into functional cardiomyocytes (iPSC-CMs). (FIG. 5A) After plating on glass-bottom dishes, RyR2^{S40R/WT} and WT iPSC-CMs were loaded with the fluorescent Ca²⁺ indicator (Fluo-4) and electrically paced at 1 Hz for 10 seconds. The appearance of abnormal Ca²⁺ release events (aCREs) after the cessation of pacing is indicative of deranged Ca²⁺ signaling in CPVT iPSC-CMs (right panel). Raw data in blue and filtered data in orange overlay. (FIG. 5B) Quantification of aCREs in wildtype (WT) and RyR2^{S40R/WT} iPSC-CMs with pre-incubation of 2 μM ruxolitinib compared to vehicle alone (DMSO). Automated analysis of Ca²⁺ transient parameters during 1 Hz pacing of iPSC-CMs for (FIG. 5C) mean amplitude, (FIG. 5D) Ca²⁺ transient duration at 50% of peak amplitude (TCa₅₀), (FIG. 5E) Upstroke velocity, and (FIG. 5F) downstroke velocity. The number of analyzed cells (n) is annotated on each graph. Statistics were performed by one-way ANOVA (non-parametric) and Kruskal-Wallis multiple comparisons test: ns=p>0.1, *p<0.05, **p<0.01, ***p<0.005, ****p<0.0001 multiple comparisons test.

FIGS. 6A-6E demonstrate that ruxolitinib prevents the arrhythmia phenotype in a murine model of CPVT. Adult murine cardiomyocytes (CMs) were isolated by retrograde perfusion of collagenase from WT and CPVT mice with the pathogenic mutation RyR2^{R176Q/WT}. (FIG. 6A) After loading with Rhod-2, cells were paced for 30 seconds at 1 Hz and the emergence of spontaneous aCREs after the cessation pacing was recorded. (FIG. 6B) Effects of pre-incubation with 2 μM ruxolitinib (or DMSO) on the frequency of aCREs for WT and RyR2R176Q/WT CMs. (FIG. 6C) To investigate the effect of ruxolitinib on arrhythmia induction, mice were treated with ruxolitinib (75 mg/kg) or vehicle by intraperitoneal injection. Ten minutes after drug treatment, an octopolar electrophysiology (EP) catheter inserted in the right ventricle, delivered programmed ventricular stimulation. (FIG. 6D) Concurrent stimulation with isoproterenol (2 mg/kg) and epinephrine (4 mg/kg) induced ventricular arrhythmias in RyR2R176Q/WT animals with vehicle but not after treatment with ruxolitinib. (FIG. 6E) Quantification of the duration of arrhythmias induced by ventricular pacing. Statistics were performed by one-way ANOVA (non-parametric) and Kruskal-Wallis multiple comparisons test, for continuous variables or Chi-Squared for discrete variables: ns=p>0.1, *p<0.05, **p<0.01, ***p<0.005, ****p<0.0001.

FIGS. 7A-7E demonstrate that ruxolitinib prevents and rescues atrial fibrillation (AF) in mice. (FIG. 7A) Representative type 1 diabetic mouse tracings immediately after atrial burst pacing. DMSO treated mice demonstrate atrial fibrillation (upper panel), whereas 75 mg/kg ruxolitinib (Rux) treated mice (single dose 10 minutes prior, intraperitoneal) retain sinus rhythm (lower panel). (FIG. 7B) Atrial fibrillation (AF) inducibility percentage from mice in FIG. 7A. Number of mice analyzed per group shown in each column: 9/16 vehicle treated mice had AF vs. 2/15 Rux-treated mice. (FIG. 7C) Sequential atrial fibrillation (AF) inducibility in OGT-transgenic mice. Mice were treated with vehicle immediately prior to AF induction and then treated with ruxolitinib (75 mg/kg) prior to pacing for a second time. Number of mice analyzed per group shown in each column (Vehicle=5/5 had AF, Rux=1/5 had AF). (FIG. 7D) Percent time in AF or sinus rhythm after ruxolitinib 75 mg/kg treatment in CREM-IbAC-X mice (n=10, paired). (FIG. 7E) Percent time in AF in individual CREM-IbAC-X mice after treatment with vehicle vs ruxolitinib 24 hours later. Statistical comparisons were performed using 2-tailed Fischer's exact test (FIGS. 7B, 7C); two-way ANOVA with Sidak's multiple comparison's test (FIG. 7D); paired Student's T-test (FIG. 7E); *p<0.05, ****p<0.0001.

FIGS. 8A-8G demonstrate that ruxolitinib does not impair short term or spatial memory in mice. (FIG. 8A) Wild type C57BL/6J mice were treated with single dose (1 hour prior) or multiple doses (7 days, twice daily) of ruxolitinib prior to behavioral testing via the novel object recognition test (NORT) and the Y maze spatial recognition memory test. (FIGS. 8B, 8C) Percent of time spent with novel object. (FIGS. 8D, SE) Total distance traveled while in testing chamber. (FIGS. 8F, 8G) Percent of time spent in novel arm of Y maze. Each data point represents a mouse, n=9-10 mice for all conditions. ns=p>0.05, *p<0.05; significance determined by one-way ANOVA and Tukey's multiple comparisons test.

FIGS. 9A-9H demonstrate the CaMKAR development and validation. (FIG. 9A) Screening to identify peptide that renders sensor sensitive to CaMKIIδ: plate reader fluorometry of 293T cells transfected with CaMKIIδ (CA=constitutively active, T287D mutation; KD=kinase dead, K43M mutation) and prototype sensors. Each peptide was fused to the 5′ end of kinase sensing cpGFP-FHA1. Sensor signal (R) defined as ratio of 488-nm-excited intensity divided by 405-nm-excited intensity; R₀=R in control condition or baseline. N=3 wells per observation. (B) Maximal stimulation of CaMKAR in 293T cells by expression of CaMKII^{T87D} plus ionomycin treatment (5 μM; n=3 wells) or untreated (n=3 wells). (FIG. 9C) Intensiometric CaMKAR signal (Ex. 488 nm, Em. 520 nm) after vehicle (Veh, n=2,488-2,615 cells) or ionomycin stimulation (n=1,733-2,220 cells), from same dataset as FIG. 1C. (FIG. 9D) Structure of CaMKII inhibitor AS100397. (FIG. 9E) In vitro CaMKII kinase assay (HotSpot substrate phosphorylation) with varying concentrations of AS100397 and ATP. (FIG. 9F) CaMKAR signal in 293T cells pre-treated with vehicle (n=1,746-1,930 cells) or Calyculin A (10 nM; n=1,051-1,595), then stimulated with ionomycin. (FIG. 9G) CaMKAR response in control (CTRL; n=151) cells or after transfection of constitutively active CaMKIIα (n=194 cells), β (n=229), δ (n=201), or γ (n=194) and (H) control (CTRL; n=772) vs constitutively active CaMKIIδ splice variants 9 (n=1,136) and B (n=1,140). Data shown as mean±SEM (unless too small to display). All observations done in biological triplicate. ns=p>0.05, ****p<0.0001; significance was determined via one-way ANOVA and Sidak's multiple comparisons test (FIG. 9A) or Dunnett's multiple comparisons test (FIGS. 9G and 9H); two-way ANOVA and Sidak's multiple comparison test (FIG. 9F); unpaired Student's T-test (FIG. 9B).

FIGS. 10A-10M demonstrate the CaMKAR benchmarking and specificity. (FIG. 10A) CaMKAR and Camui-NR3 signal response to ionomycin in 293T cells: assayed for signal-to-noise ratio (CaMKAR n=447 cells; Camui n=469 cells), and (FIG. 10B) activation kinetics (CaMKAR n=73 cells; Camui n=3,576-4,147 cells). (FIG. 10C) CaMKAR activity tracking in rat hippocampal neurons pre-treated with CaMKII inhibitors and then stimulated with ionomycin (added at 2 minute mark). (FIG. 10D) Normalized mean sensor signal across all timepoints post-stimulation in FIG. 10C. Three baseline values were captured prior to ionomycin stimulation; datapoints represent individual cells. (FIG. 10E) Camui activity tracking in rat hippocampal neurons pre-treated with CaMKII inhibitors and then stimulated with ionomycin (added at 2 minute mark). (FIG. 10F) Normalized mean sensor signal across all timepoints post-stimulation in FIG. 10E. Three baseline values were captured prior to ionomycin stimulation; datapoints represent individual cells. (FIG. 10G) Effect size comparison of KN-93 and AS100397 for each sensor. (FIG. 10H) Sensor-expressing rat hippocampal neurons stimulated with ionomycin and tracked for sensor activity over time. (FIG. 10I) Maximal signal change from FIG. 10H. (FIG. 10J) Left: CaMKAR-expressing neonatal ventricular cardiomyocytes (NRVMs) were pretreated with AS100397 (10 μM, n=722-749 cells), anti-CAMKIIδ siRNA (n=665-700) or scrambled siRNA (n=571-594) and stimulated with 2 Hz pacing (arrow). Right: validation of anti-CaMKIIδ siRNA (immunoblot and densitometry) in technical triplicate from the same cell preparation as left. (FIG. 10K) 293T cells expressing PKA sensor ExRai-AKAR2 treated with vehicle (n=1,066-1,092 cells) or Fsk (50 μM)/IBMX (100 μM)(n=908-1,135 cells). Treatment addition at arrow. (FIG. 10L) 293T cells expressing PKC sensor ExRai-CKAR treated with vehicle (n=1,490-1,591 cells), PMA (100 ng/mL; n=932-970), or PMA+G6 6976 (500 nM; n=770-823). Treatment addition at arrow. (FIG. 10M) Effect of vehicle (n=722-792) or pre-treatment with G6 6976 (500 nM; n=509-587) on ionomycin CaMKAR response. Data shown as mean±SEM (unless too small to display). All observations done in biological triplicate. ns=p>0.05,**p<0.01, ****p<0.0001; significance was determined via unpaired Student's T-test (FIG. 10A); nonlinear regression (FIG. 10B); one-way ANOVA and Dunnet's multiple comparison test (FIGS. 10D, 10 F, and 10I); two-way ANOVA and Sidak's multiple comparisons test (FIGS. 10G, 10K, and 10M) and Tukey's multiple comparisons test (FIGS. 10J and 10L).

FIGS. 11A-11E demonstrate that CaMKAR is functional in vitro. Purified recombinant CaMKAR was co-incubated with CaMKIIδc, Ca²⁺/CaM±ATP and assayed via fluorescence plate reader for (FIG. 11A) excitation spectrum at 520 nm emission, (FIG. 11B) emission spectrum at 400 nm excitation, (FIG. 11C) emission spectrum at 488 nm excitation, and (FIG. 11D) maximal CaMKAR ratio (see methods). (FIG. 11E) Purified CaMKAR co-incubated with increasing concentrations of purified CaMKIIδc (see methods). Data shown as mean±SEM (unless error is too small to graph). Observations done in technical triplicate (FIGS. 11A-11D) or single replicate (FIG. 11E).****=p<0.0001; significance determined via unpaired two-tailed t-test.

FIGS. 12A-12B demonstrate that K562 cells tolerate CaMKIIδ^{T287D} overexpression and are suitable for screening. K562 cells infected with lentiviruses encoding CMV-driven CaMKAR and TetON-driven kinase-dead or constitutively active CaMKIIδc were treated with doxycycline for 24 hours and assayed for viability via CellTiterGlo 2.0 luminescence. Cells displayed doxycycline-associated toxicity but tolerated hyperactive CaMKII. Observations done in biological triplicate. Data points represent individual wells normalized to untreated condition. ns=p<0.05, *p<0.05; significance determined via two-way ANOVA and Tukey's multiple comparisons test. (FIG. 12B) CaMKAR signal in K562^{CaMKAR/CaMKII} cells co-incubated with AS100397 (10 μM, n=240 wells) or vehicle (n=504 wells) in 384 well plate format. Data minmax normalized to 0-100% based on means of the two groups. ****p<0.0001; significance determined via unpaired Student's t-test.

FIG. 13 demonstrates the CaMKII inhibition and cell viability in 293T cells. CaMKAR- and CaMKII^{T287D}-expressing 293T cells were treated with hits identified in FIG. 3C, AS100397 as positive control. Compounds were incubated for 12 hours and assayed via high content imaging for CaMKII activity (black) and via CellTiter-Glo 2.0 luminescence for cell viability (red). Data represent mean±SEM from 3 independent wells with overlayed dose-response IC₅₀ curves.

FIG. 14 shows the chemical structures of identified CaMKII inhibitors.

FIGS. 15A-15D demonstrates that CaMKII inhibition is independent of JAK1/2 inhibition. (FIG. 15A) Known JAK1/2 inhibitors included in the primary screen (from FIG. 2B) are displayed in red (analyzed drugs shown on right). (FIG. 15B) JAK1/2 inhibitors from from FIG. 15A plotted by their known in vitro IC50 against JAK1 and JAK2 versus their CaMKII inhibition score. Insert: linear regression analysis to determine significance of correlation. (FIG. 15C) Pacing-induced CaMKII activity (arrow) in CaMKAR-expressing NRVMs pre-treated with vehicle (n=284-354) JAK inhibitor filgotinib (0.5 μM; n=291-335; 2 μm n=290-330 cells). (FIG. 15D) In vitro kinase activity assay (HotSpot radioactive phosphorous substrate labeling) was performed with varying concentrations of ruxolitinib and ATP. As ATP concentration increases, the apparent IC₅₀ of ruxolitinib also increases, consistent with ATP-competition.

FIGS. 16A-16E comparing other top candidates against ruxolitinib. Human iPSCs with the pathogenic mutation RyR2^{S40R/WT} were differentiated into cardiomyocytes and loaded with Rhod-2 as a Ca²⁺ indicator. Each test compound was pre-incubated for >10 minutes at a concentration of 2 μM. Cells were paced at 1 Hz for 10 s with continuation of high-speed Ca²⁺ imaging after the cessation of pacing to detect (FIG. 16A) abnormal Ca²⁺ release events. Automated analysis of paced Ca²⁺ transients revealed the (FIG. 16B) peak amplitude, (FIG. 16C) upstroke velocity, (FIG. 16D) Downstroke velocity, and (FIG. 16E) Ca²⁺ transient duration measured at 50% of the peak (TCa₅₀). The number of analyzed cells (n) for each parameter is annotated on E. Statistics were performed by one-way ANOVA (nonparametric) against ruxolitinib and Kruskal-Wallis multiple comparisons test: ns=p>0.1, *p<0.05, ****p<0.0001.

FIGS. 17A-17C demonstrate that Ruxolitinib inhibits adrenergically induced increases in heart rate. Wild type (WT) and RyR2^{R176Q/WT} animals were instrumented with a 2 lead ECG to monitor heart rate. Prior to stimulation, animals were treated with ruxolitinib (75 mg/kg) or vehicle only (DMSO) by intraperitoneal injection followed by Isoproterenol (ISO; 4 mg/kg) or epinephrine (EPI; 2 mg/kg). Continuous monitoring of the heart rate in (FIG. 17A) WT or (FIG. 17B) RyR2^{R176Q/WT} animals, demonstrated the inhibitory effects of ruxolitinib. (FIG. 17C) The fractional change from baseline was calculated for each animal and compared for each condition. Statistics were performed by one-way ANOVA (non-parametric) and Kruskal-Wallis multiple comparisons test: ns=p>0.1, *p<0.05, **p<0.01, ****p<0.0001.

FIGS. 18A and 18B demonstrate that ruxolitinib inhibits CaMKII in CPVT mice. (FIG. 18A) Whole heart lysates prepared from WT and RyR2^{R176Q/WT} animals treated with ruxolitinib and/or isoproterenol (ISO) were separated by SDS-page electrophoresis and probed with specific antibodies to vinculin, phospholamban (PLN), phosphorylated phospholamban (pPLN), and GAPDH. (FIG. 18B) Quantification of immunoblot from FIG. 18A. Data points represent individual mice. Statistics were performed by one-way ANOVA (non-parametric) and Kruskal-Wallis multiple comparisons test: ns=p>0.1, *p<0.05, **p<0.01, ***p<0.005, ****p<0.0001.

FIGS. 19A and 19B demonstrate that ruxolitinib inhibits CaMKII in atria. (FIG. 19A) Atrial heart lysates prepared from type 1 diabetic (T1D) animals treated with ruxolitinib (Rux) or vehicle (Ctrl) were separated by SDS-page electrophoresis and probed with specific antibodies to phospholamban (PLN), phosphorylated phospholamban (pPLN). P=pentameric PLN; M=monomeric PLN. (FIG. 19B) Quantification of immunoblot from FIG. 19A. Aggregate of both pentameric and monomeric bands. Data points represent individual mice. Significance was determined via unpaired T-test; ****p<0.0001.

FIGS. 20A-20E demonstrate that ruxolitinib does not impair performance in the Barnes maze test. (FIG. 20A) Wild type C57BL/6J mice were treated with intraperitoneal ruxolitinib twice daily starting 1 day prior to longitudinal training in the Barnes maze. Mice received 4 training sessions and one probe session (day 6 of ruxolitinib treatment). (FIG. 20B) Latency to escape the Barnes maze during the probe trial (day 6). There was no significant deficit in ruxolitinib (Rux) treated mice over vehicle (Veh). Datapoints represent individual mice. (FIGS. 20C-20E) Latency to escape maze, primary errors, and total errors of mice subjected to Barnes maze during the 4 training trials. Statistical testing shown below each graph with proportion and significance of variance by each parameter. Data shown as mean±SEM. Significance was determined via Student's T-test (FIG. 20B), or two-way ANOVA (FIGS. 20C-20E).

FIG. 21 is a schematic representation of the overall CaMKAR design and function.

FIG. 22A is a plot demonstrating substrate screening to identify a sensor that is sensitive to CaMKII. HEK293 cells were transfected with candidate sensor designs and stimulated with constitutively active (T287D) or kinase dead (K43M) CaMKII. Of note, ExRai-AKAR2 is very sensitive to CaMKII activity.

FIG. 22B is a graph demonstrating that ExRai-AKAR2, but not CaMKAR, responds to PKA activation (Fsk/IBMX). R/R is the fluorescence ratio between the two CaMKAR channels; ratio is normalized to baseline.

FIGS. 23A and 23B demonstrate that CaMKAR sensitively responds to chemical and genetic CaMKII activation. FIG. 23A: HEK293 cells transfected with CaMKAR-encoding plasmid were exposed to Ionomycin (Ca²⁺ overload) or vehicle. FIG. 23B: HEK293 cells transfected with CaMKAR-encoding plasmid were co-transfected with constitutively active (T287D) or kinase-dead (K43M) CaMKII. CaMKAR activity was determined via fluorescence microscopy in FIG. 23A and plate reader in FIG. 23B. R/R₀ is the fluorescence ratio between the two CaMKAR channels; ratio is normalized to baseline.

FIGS. 24A-24E are a series of plots demonstrating that CaMKAR is superior to previous CaMKII sensor “Camui” in terms of Dynamic range, signal-to-noise ratio, and time-on kinetics. HEK293 cells transfected with CamuiNR3-encoding or CaMKAR-encoding plasmid were exposed to Ionomycin (Ca²⁻ overload) or vehicle and sensor signal was tracked via fluorescence microscopy.

FIG. 25 is a series of images of fluorescent stains demonstrating that CaMKAR can be introduced to create a stable cell line via Lentivirus. Additionally, CaMKAR can be directed to specific cellular compartments by attaching different localizing tags. HEK293 cells were infected with CaMKAR lentivirus expressing differing tags and assayed via 100× objective fluorescence microscopy. NES=cytosol localizing tag, NLS=nucleus localizing tag, mito=mitochondria localizing tag.

FIG. 26 is a graph demonstrating that Purified recombinant CaMKAR is fully functional and sensitive to CaMKII in microplate reader assay. Purified CaMK AR signal becomes weaker at 405 nm excitation and stronger at 488 nm excitation after incubation with CaMKII (this is the basis for ratiometric signal R/Rₒ).

FIG. 27 (includes FIGS. 27A-27D) and FIG. 28 depict results that show CaMKAR is insensitive to activators of related serine-threonine kinases, CaMKI, CaMKIV, PKA, PKD and PKC.

### DETAILED DESCRIPTION

The Ca²⁺ and Calmodulin dependent protein kinase II (CaMKII) is a known driver of heart injury, promoting heart failure, arrhythmias and death. There is great interest to further understand CaMKII signaling in the heart and to make CaMKII inhibitors to prevent heart injury and death. Both of these goals have been hindered by the lack of tools for measuring CaMKII activity with precise temporal and subcellular resolution in living cells.

### Biosensors

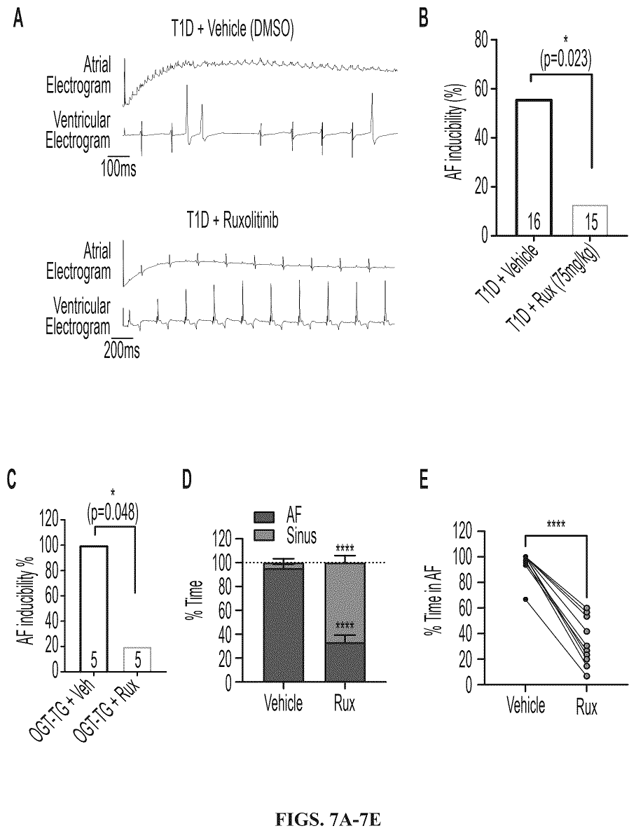

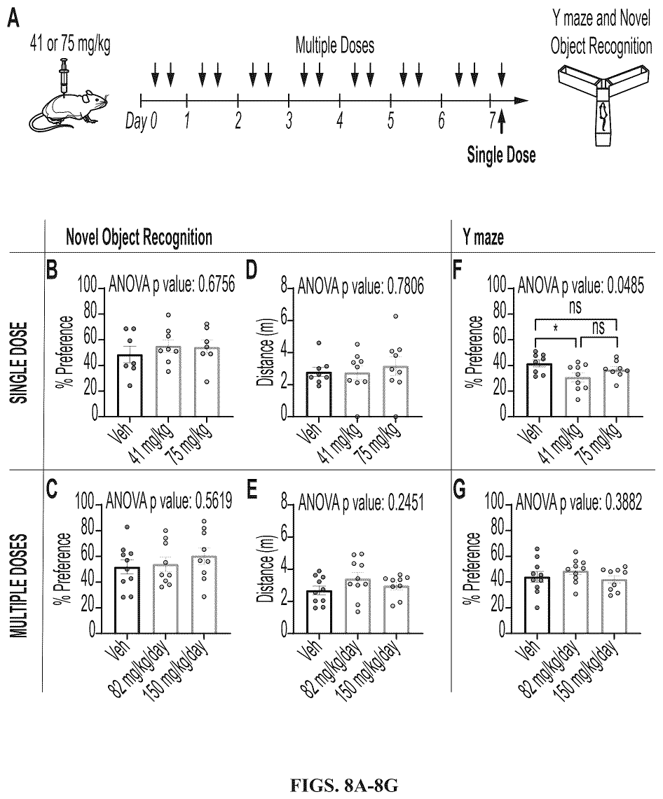

To address this need, the present disclosure is based on the discovery of a CaMKAR (CaMKII Activity Reporter) a novel, genetically encoded fluorescent biosensor that reports CaMKII activity in living cells and in vitro. The biosensor is comprised of an amino acid sequence containing a CaMKII substrate, a fluorescent protein, and a phospho-amino acid binding protein. When CaMKII is active, it phosphorylates the substrate, which causes the fluorescent protein to become detectably brighter. The biosensor signal is reported as a ratio collected at two wavelengths (405; which decreases in active state, and 488: which increases). The results show that this phosphorylation is due to CaMKII activity and triggers increased fluorescence that is measurable by microscopy, plate reader, and flow cytometry. The biosensor sensitively reports CaMKII activity using pharmacological (Ionomycin-mediated Ca²⁺ overload) and genetic (CaMKIIT287D, constitutively active mutation) activators of CaMKII. The instantly described biosensor has vastly superior dynamic range, signal-to-noise ratio, and activation kinetics compared to currently available sensors. Furthermore, the results show that CaMKAR is insensitive to activators of related serine-threonine kinases, CaMKI, CaMKIV, PKA and PKC. In addition, by attaching different localizing signals to CaMKAR, the biosensor can be restricted to specific subcellular compartments. See FIGS. 7 and 8.

Accordingly, in certain embodiments, the synthetic biosensor comprises: an enzyme substrate, a detectably labelled protein, and a phospho-amino acid binding protein. In certain embodiments, the enzyme substrate is a kinase. In certain embodiments, the kinase is calcium/calmodulin-dependent protein kinase II (CaMKII). In certain embodiments, the CaMKII substrate comprises an amino acid sequence having at least about 70% sequence identity to MHRQETVDCLK (SEQ ID NO: 1). In certain embodiments, the CaMKII substrate comprises an amino acid sequence having at least about 90% sequence identity to MHRQETVDCLK (SEQ ID NO: 1). In certain embodiments, the CaMKII substrate comprises an amino acid sequence having at least about 95% sequence identity to MHRQETVDCLK (SEQ ID NO: 1). In certain embodiments, the CaMKII substrate comprises the amino acid sequence MHRQETVDCLK (SEQ ID NO: 1).

In certain embodiments, the biosensor comprises a calcium/calmodulin-dependent protein kinase II (CaMKII) substrate comprising an amino acid sequence having at least about 70% sequence identity to X_{N+1}MHRQETVDCLK Y_{N+1} (SEQ ID NO: 2) wherein X or Y comprise amino acids analogs or variants thereof and N=0, 1, 2, 3, 4 or more. In certain embodiments, the CaMKII substrate comprises an amino acid sequence having at least about 90% sequence identity to SEQ ID NO: 2. In certain embodiments, the CaMKII substrate comprises an amino acid sequence having at least about 95% sequence identity to SEQ ID NO: 2. In certain embodiments, the CaMKII substrate comprises the amino acid sequence SEQ ID NO: 2.

In certain embodiments, a calcium/calmodulin-dependent protein kinase II (CaMKII) substrate comprises an amino acid sequence having at least about 70% sequence identity to MHRQETVDCLK (SEQ ID NO: 1). In certain embodiments, the CaMKII substrate comprises an amino acid sequence having at least about 90% sequence identity to MHRQETVDCLK (SEQ ID NO: 1). In certain embodiments, the CaMKII substrate comprises an amino acid sequence having at least about 95% sequence identity to MHRQETVDCLK (SEQ ID NO: 1). In certain embodiments, the CaMKII substrate comprises the amino acid sequence MHRQETVDCLK (SEQ ID NO: 1).

In certain embodiments, a calcium/calmodulin-dependent protein kinase II (CaMKII) substrate comprising an amino acid sequence having at least about 70% sequence identity to X_{N+1}MHRQETVDCLK Y_{N+1} (SEQ ID NO: 2) wherein X or Y comprise amino acids analogs or variants thereof and N=0, 1, 2, 3, 4 or more. In certain embodiments, the CaMKII substrate comprises an amino acid sequence having at least about 90% sequence identity to SEQ ID NO: 2. In certain embodiments, the CaMKII substrate comprises an amino acid sequence having at least about 95% sequence identity to SEQ ID NO: 2. In certain embodiments, the CaMKII substrate comprises the amino acid sequence SEQ ID NO: 2.

In certain embodiments, the components are linked together to provide a unimolecular biosensor. In certain embodiments, the components are covalently attached by a linker, preferably a flexible polypeptide linker. In an embodiment, the flexible polypeptide linker has a length corresponding to the length of a random amino acid sequence of about 50 to about 500-1000 amino acids, for example corresponding to the length of a random amino acid sequence of about 100 to about 400-500 amino acids, preferably about 200-400 amino acids, for example about 300. In a further embodiment, the flexible linker comprises a random amino acid sequence of about 50 to about 500-1000 amino acids, for example a random amino acid sequence of about 100 to about 400-500 amino acids, preferably a random amino acid sequence of about 200-400 amino acids, for example about 300 amino acids. Methods for designing flexible amino acid linkers, and more specifically linkers with minimal globularity and maximal disorder, are known in the art. This may be achieved, for example, using the Globplot 2.3 program. The sequence may be further optimized to eliminate putative aggregation hotspots, localization domains, and/or interaction and phosphorylation motifs.

In certain embodiments, the biosensor comprises one or more amino acid variants. The variant as used herein refers to a protein/polypeptide having has an identity or similarity of at least 60% with a reference (e.g., native) sequence and retains a desired activity thereof, for example the capacity to bind to a target protein and/or to translocation to a cellular compartment. In further embodiments, the variant has a similarity or identity of at least 65, 70, 75, 80, 85, 90, 91, 92, 93, 94, 95, 96, 97, 98 or 99% with a reference (e.g., native) sequence and retains a desired activity thereof. “Similarity” and “identity” refers to sequence similarity/identity between two polypeptide molecules. The similarity or identity can be determined by comparing each position in the aligned sequences. A degree of similarity or identity between amino acid sequences is a function of the number of matching or identical amino acids at positions shared by the sequences. Optimal alignment of sequences for comparisons of similarity or identity may be conducted using a variety of algorithms, such as the local homology algorithm of Smith and Waterman, 1981, Adv. Appl. Math 2: 482, the homology alignment algorithm of Needleman and Wunsch, 1970, J. Mol. Biol. 48: 443, the search for similarity method of Pearson and Lipman, 1988, Proc. Natl. Acad. Sd. USA 85: 2444, and the computerized implementations of these algorithms (such as GAP, BESTFIT, FASTA and TFASTA in the Wisconsin Genetics Software Package, Genetics Computer Group, Madison, Wis., U.S.A.). Sequence similarity or identity may also be determined using the BLAST algorithm, described in Altschul et al., 1990, J. Mol. Biol. 215: 403-10 (using the published default settings). Software for performing BLAST analysis may be available through the National Center for Biotechnology Information web site (ncbi.nlm.nih.gov/).

The term “amino acid” as used herein refers to naturally occurring and synthetic α, β, γ, and δ amino acids, and includes but is not limited to, amino acids found in proteins, i.e. glycine, alanine, valine, leucine, isoleucine, methionine, phenylalanine, tryptophan, proline, serine, threonine, cysteine, tyrosine, asparagine, glutamine, aspartate, glutamate, lysine, arginine and histidine. Alternatively, the amino acid can be a derivative of alanyl, valinyl, leucinyl, isoleucinyl, prolinyl, phenylalaninyl, tryptophanyl, methioninyl, glycinyl, serinyl, threoninyl, cysteinyl, tyrosinyl, asparaginyl, glutaminyl, aspartoyl, glutaroyl, lysinyl, argininyl, histidinyl, β-alanyl, β-valinyl, β-leucinyl, β-isoleucinyl, β-prolinyl, β-phenylalaninyl, β-tryptophanyl, β-methioninyl, β-glycinyl, β-serinyl, β-threoninyl, β-cysteinyl, β-tyrosinyl, β-asparaginyl, β-glutaminyl, β-aspartoyl, β-glutaroyl, β-lysinyl, β-argininyl or β-histidinyl. When the term amino acid is used, it is considered to be a specific and independent disclosure of each of the esters of a, β, γ, and δ glycine, alanine, valine, leucine, isoleucine, methionine, phenylalanine, tryptophan, proline, serine, threonine, cysteine, tyrosine, asparagine, glutamine, aspartate, glutamate, lysine, arginine and histidine in the D and L-configurations.

Accordingly, the amino acids in the biosensors can include natural and synthetic amino acid substitutions.

Detectable Label: Any fluorescent polypeptide (also referred to herein as a fluorescent label) well known in the art is suitable for use as a domain of the subject biosensor polypeptides of the present invention. A suitable fluorescent polypeptide will be one that can be expressed in a desired host cell, such as a mammalian cell, and will readily provide a detectable signal that can be assessed qualitatively (positive/negative) and quantitatively (comparative degree of fluorescence). Exemplary fluorescent polypeptides include, but are not limited to, yellow fluorescent protein (YFP), cyan fluorescent protein (CFP), GFP, mRFP, RFP (tdimer2), HCRED, etc., or any mutant (e.g., fluorescent proteins modified to provide for enhanced fluorescence or a shifted emission spectrum), analog, or derivative thereof. Further suitable fluorescent polypeptides, as well as specific examples of those listed herein, are provided in the art and are well known.

In some embodiments where multiple biosensor polypeptides are present in a cell, the fluorescent polypeptide of the biosensor is selected so that each biosensor polypeptide in the cell has a detectably different emission spectrum. For example, in such embodiments, the G1BP fluorescent label, the SBP fluorescent label, and the MBP fluorescent label, when present in the same cell, are designed to have detectably distinct emission spectra to facilitate detection of a distinct signal from each biosensor (e.g., through use of different filters in the imaging system).

In certain embodiments, the label may be radioactive. Some examples of useful radioactive labels include ³²P, ¹²⁵I, ¹³¹I, and ³H. Use of radioactive labels have been described in U.K. 2,034,323, U.S. Pat. Nos. 4,358,535, and 4,302,204. Some examples of non-radioactive labels include enzymes, chromophores, atoms and molecules detectable by electron microscopy, and metal ions detectable by their magnetic properties.

Some useful enzymatic labels include enzymes that cause a detectable change in a substrate. Some useful enzymes and their substrates include, for example, horseradish peroxidase (pyrogallol and o-phenylenediamine), β-galactosidase (fluorescein β-D-galactopyranoside), and alkaline phosphatase (5-bromo-4-chloro-3-indolyl phosphate/nitro blue tetrazolium). The use of enzymatic labels has been described in U.K. 2,019,404, EP 63,879, and by Rotman, Proc. Natl. Acad. Sci. USA, 47, 1981-1991 (1961).

Useful chromophores include, for example, fluorescent, chemiluminescent, and bioluminescent molecules, as well as dyes. Some specific chromophores useful in the present disclosure include, for example, fluorescein, rhodamine, Texas red, phycoerythrin, umbelliferone, luminol.

The labels may be conjugated to the biosensor by methods that are well known in the art. The labels may be directly attached through a functional group on the probe. The probe either contains or can be caused to contain such a functional group. Some examples of suitable functional groups include, for example, amino, carboxyl, sulfhydryl, maleimide, isocyanate, isothiocyanate. Alternatively, labels such as enzymes and chromophores may be conjugated to the antibodies or nucleotides by means of coupling agents, such as dialdehydes, carbodiimides, dimaleimides, and the like.

In certain embodiment, the biosensors of the disclosure can be used for imaging. In imaging uses, the complexes are labeled so that they can be detected outside the body. Typical labels are radioisotopes, usually ones with short half-lives. The usual imaging radioisotopes, such as ¹²³I, ¹²⁴I, ¹²⁵I, ¹³¹I, ^{99m}TC, ¹⁸⁶Re, ¹⁸⁸Re, ⁶⁴Cu, ⁶⁷Cu, ²¹²Bi, ²¹³Bi, ⁶⁷C, ⁹⁰Y, ¹¹¹In, ¹⁸F, ³H, ¹⁴C, ³⁵S or ³²P can be used. Nuclear magnetic resonance (NMR) imaging enhancers, such as gadolinium-153, can also be used to label the complex for detection by NMR. Methods and reagents for performing the labeling, either in the polynucleotide or in the protein moiety, are considered known in the art.

Reporter genes useful in the present disclosure include acetohydroxy acid synthase (AHAS), alkaline phosphatase (AP), beta galactosidase (LacZ), beta glucoronidase (GUS), chloramphenicol acetyltransferase (CAT), green fluorescent protein (GFP), red fluorescent protein (RFP), yellow fluorescent protein (YFP), cyan fluorescent protein (CFP), horseradish peroxidase (HRP), luciferase (Luc), nopaline synthase (NOS), octopine synthase (OCS), and derivatives thereof. Multiple selectable markers are available that confer resistance to ampicillin, bleomycin, chloramphenicol, gentamycin, hygromycin, kanamycin, lincomycin, methotrexate, phosphinothricin, puromycin, and tetracycline. Methods to determine modulation of a reporter gene are well known in the art, and include, but are not limited to, fluorometric methods (e.g. fluorescence spectroscopy, Fluorescence Activated Cell Sorting (FACS), fluorescence microscopy), antibiotic resistance determination.

Localization Sequence: The term “localization sequence” refers to a biomolecule, such as a polypeptide or peptide, which, when attached to the synthetic biosensor embodied herein (as a fusion protein, for example), targets them to a particular compartment, organelle or localization within the cell, such as for example the plasma membrane (or a particular subdomain of the plasma membrane, such as lipid rafts), the endosomes (e.g. early and/or late endosomes), the lysosomes, the phagosomes, the ribosomes, the mitochondria, the endoplasmic reticulum, the Golgi apparatus, the nucleus, etc. Peptides that target proteins to specific compartment, organelle or localization within the cell are known in the art and include endoplasmic reticulum (ER) signal peptide or ER-retrieval sequence, nuclear localization signal (NLS) peptide, and mitochondrial localization signal (MLS) peptide, for example.

In certain embodiments, the localization sequence is a plasma membrane (PM) targeting sequence. Any localization sequence capable of recruiting the biosensor to the PM may be used in the biosensors. The biosensor may thus be fused to any protein found at the plasma membrane (e.g., receptors or any other protein found at the PM), or fragments thereof. Examples or localization sequences include peptides/polypeptides comprising a signal sequence for protein lipidation/fatty acid acylation, such as myristoylation, palmitoylation and prenylation, as well as polybasic domains. Several proteins are known to be myristoylated, palmitoylated and/or prenylated (e.g., protein kinases and phosphatases such as Yes, Fyn, Lyn, Lck, Hck, Fgr, G, proteins, nitric oxide synthase, ADP-ribosylation factors (ARFs), calcium binding proteins and membrane or cytoskeleton-associated structural proteins such as MARCKS (see, e.g., Wright et al., J Chem Biol. March 2010; 3(1): 19-35; Alcart-Ramos et al., Biochimica et Biophysica Acta (BBA)—Biomembranes, Volume 1808, Issue 12, December 2011, Pages 2981-2994), and thus the myristoylation, palmitoylation and prenylation signal sequences from any of these proteins may be used in the biosensor. In certain embodiments, the myristoylation and/or palmitoylation sequence is from the Lyn kinase.

In certain embodiments, the localization sequence comprises a cytokine sequence which can bind to its receptor, e.g., IL-2 or comprises a receptor sequence which binds to the cytokine. In other embodiments, the localization sequence can be an aptamer or binding fragment of an antibody, e.g., scFv.

In certain embodiments, the localization sequence is an endosomal targeting moiety. Several endosomal targeting moieties/markers are known in the art and include the Rab family of proteins (RAB4, RAB5, RAB7, RAB9 and RAB11), mannose 6-phosphate receptor (M6PR), caveolin-1 and -2, transferrin and its receptor, clathrin, as well as proteins comprising a FYVE domain such as early endosome autoantigen 1 (EEA1), Rabenosyn-5, Smad anchor for receptor activation (SARA), Vps27p and Endofin. Some markers are more specific to early endosomes (e.g., RAB4, Transferrin and its receptor, and proteins comprising a FYVE domain), others are more specific to late endosomes (e.g., RAB7, RAB9, and M6PR) and others are more specific to recycling endosomes (e.g., RAB11, RAB4). Thus, these proteins or suitable fragments thereof may be fused to the synthetic biosensor embodied herein to link/target them to an endosomal localization.

In certain embodiments, the localization sequence is a lysosomal targeting moiety, such as for example LAMP1 and LAMP2. Thus, these proteins or suitable fragments thereof may be fused to the synthetic biosensor embodied herein to link/target them to a lysosomal localization.

In certain embodiments, the localization sequence is a peroxisomal targeting moiety, such as for example PMP70, PXMP2 and Catalase. Thus, these proteins or suitable fragments thereof may be fused to the synthetic biosensor embodied herein to link/target them to a peroxisomal localization.

In certain embodiments, the localization sequence is an autophagosomal targeting moiety, such as for example ATG (AuTophaGy related) family proteins (ATG4, ATG5, ATG16, ATG12, see Lamb et al., Nature Reviews Molecular Cell Biology 14, 759-774 (2013)), LC3A/B and SQSTMI/p62. Thus, these proteins or suitable fragments thereof may be fused to the synthetic biosensor embodied herein to link/target them to an autophagosomal localization.

In certain embodiments, the localization sequence is a ribosome targeting moiety. Several endosomal targeting moieties/markers are known in the art and include the Ribosomal Proteins (L7a, S3 and S6). Thus, these proteins or suitable fragments thereof may be fused to the synthetic biosensor embodied herein to link/target them to a ribosomal localization.

In certain embodiments, the localization sequence is an endoplasmic reticulum (ER) targeting moiety. Several ER targeting moieties/markers are known in the art and include ERp72, ERp29, Protein disulphide isomerase (PDI), HSP70 family proteins such as GRP78 (HSPA5), GRP94 (HSP90B1) and GRP58 (PDIA3), Calnexin and Calreticulin. Thus, these proteins or suitable fragments thereof may be fused to the synthetic biosensor embodied herein to link/target them to an ER localization.

In certain embodiments, the localization sequence is a Golgi targeting moiety. Several Golgi targeting moieties/markers are known in the art and include eNOS (e.g., the N-terminal portion thereof, J. Liu et al., Biochemistry, 35 (1996), pp. 13277-13281), GM130, Golgin-97, the 58K protein, Trans-Golgi network membrane protein 2 (TGOLN2), TGN46, TGN38, Mannosidase 2, Syntaxin 6, GM130 (GOLGA2), Golgin-160, Membrin (GS27), GS28, Coatomer proteins, Rbet1 and RCAS1. Thus, these proteins or suitable fragments thereof may be fused to the synthetic biosensor embodied herein to link/target them to a Golgi apparatus localization.

In certain embodiments, the localization sequence is a mitochondria targeting moiety. Several mitochondria targeting moieties/markers are known in the art and include AIF, COX IV, Cytochrome C, hexokinase 1, SOD1, SDHA, Pyruvate dehydrogenase, VDAC, TOMM22, UCP1, UCP2, UCP3, PHB1 Galpha12 (or the N-terminal portion thereof; Andreeva et al., FASEB J. 2008 August; 22(8):2821-31. Epub 2008 Mar. 26), a protein of the BcI-family member or a fragment thereof (Mossalam et al., Mol Pharm. 2012 May 7; 9(5): 1449-1458). Thus, these proteins or suitable fragments thereof may be fused to the synthetic biosensor embodied herein to link/target them to a mitochondrial localization. The nuclear targeting moiety may also comprise a mitochondrial targeting signal, which is a 10-70 amino acid long peptide that directs newly synthesized proteins to the mitochondria. It is found at the N-terminus and consists of an alternating pattern of hydrophobic and positively charged amino acids to form an amphipathic helix. Mitochondrial targeting signals can contain additional signals that subsequently target the protein to different regions of the mitochondria, such as the mitochondrial matrix.

In certain embodiments, the localization sequence is a nuclear targeting moiety. Several nuclear targeting moieties/markers are known in the art and include Lamin A/C, Nudeoporins (NUP), ASHL2, ESET, Histones, LSDI, DNA repair enzymes such as PARP, and P84/THOC1. Thus, these proteins or suitable fragments thereof may be fused to The synthetic biosensor embodied herein to link/target them to a nuclear localization. The nuclear targeting moiety may also comprises a nuclear localization signal or sequence (NLS), which is an amino acid sequence that tags a protein for import into the cell nucleus by nuclear transport. Typically, this signal consists of one or more short sequences of positively charged lysines or arginines exposed on the protein surface. The best characterized transport signal is the classical NLS (cNLS) for nuclear protein import, which consists of either one (monopartite) or two (bipartite) stretches of basic amino acids. Monopartite cNLSs are exemplified by the SV40 large T antigen NLS and bipartite cNLSs are exemplified by the nucleoplasmin NLS.

In certain embodiments, the localization sequence is a nuclear export sequence (NES). NES is a short amino acid sequence (typically 4 hydrophobic residues) in a protein that targets it for export from the cell nucleus to the cytoplasm through the nuclear pore complex using nuclear transport. The sequence of such NES may be for example LxxxLxxLxL, where “L” is a hydrophobic residue (often leucine) and “x” is any other amino acid. In proteins that are translocated from cytosol to nucleus (such as ERK or MDM2), a decrease in the BRET signal is detected using an NES moiety.

In certain embodiments, the localization sequence is a cytoskeleton targeting moiety, for example actin or a fragment thereof, or a protein comprising an actin-binding domain (ABD), such as the N-terminal F-actin binding domain of Inositol-1,4,5-trisphosphate-3-kinase-A (ITPKA) (Johnson and Schell, Mol. Biol. Cell Dec. 15, 2009 vol. 20 no. 24 5166-5180).

The localization sequence can be fused or linked to the N- and/or C-termini of the biosensor. Other domains or linkers may be present at the N-terminal, C-terminal or within the components of the biosensor. In embodiments, the synthetic biosensor embodied herein may be covalently linked to the localization sequence either directly (e.g., through a peptide bond) or “indirectly” via a suitable linker moiety, e.g., a linker of one or more amino acids (e.g., a polyglycine linker) or another type of chemical linker (e.g., a carbohydrate linker, a lipid linker, a fatty acid linker, a polyether linker, PEG, etc. In an embodiment, one or more additional domain(s) may be inserted before (N-terminal), between or after (C-terminal) the components of the biosensor. In certain embodiments, the linker comprises about 4 to about 50 amino acids, about 4 to about 40, 30 or 20 amino acids, or about 5 to about 15 amino acids, e.g., 4, 5, 6, 7, 8, 9, 10, 11, 12, 13, 14, 15, 16, 17, 18, 19 or 20 amino acids.

Vectors: The present disclosure provides a nucleic acid encoding the biosensor. In certain embodiments, the nucleic acid is present in a vector/plasmid, in a further embodiment an expression vector/plasmid. Such vectors comprise a nucleic acid sequence capable of encoding the above-defined first and/or second component(s) operably linked to one or more transcriptional regulatory sequence(s), such as promoters, enhancers and/or other regulatory sequences.

The term “vector” refers to a nucleic acid molecule, which is capable of transporting another nucleic acid to which it has been linked. One type of vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively linked are referred to herein as “expression vectors”. A recombinant expression vector of the present invention can be constructed by standard techniques known to one of ordinary skill in the art and found, for example, in Sambrook et al. (1989) in Molecular Cloning: A Laboratory Manual. A variety of strategies are available for ligating fragments of DNA, the choice of which depends on the nature of the termini of the DNA fragments and can be readily determined by persons skilled in the art. The vectors of the present invention may also contain other sequence elements to facilitate vector propagation and selection in bacteria and host cells. In addition, the vectors of the present invention may comprise a sequence of nucleotides for one or more restriction endonuclease sites. Coding sequences such as for selectable markers and reporter genes are well known to persons skilled in the art.

A recombinant expression vector comprising a nucleic acid sequence of the present invention may be introduced into a cell (a host cell), which may include a living cell capable of expressing the protein coding region from the defined recombinant expression vector. The living cell may include both a cultured cell and a cell within a living organism. Accordingly, the invention also provides host cells containing the recombinant expression vectors of the invention. The terms “cell”, “host cell” and “recombinant host cell” are used interchangeably herein. Such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

Vector DNA can be introduced into cells via conventional transformation or transfection techniques. The terms “transformation” and “transfection” refer to techniques for introducing foreign nucleic acid into a host cell, including calcium phosphate or calcium chloride co-precipitation, DEAE-dextran-mediated transfection, lipofection, electroporation, microinjection and viral-mediated transfection. Suitable methods for transforming or transfecting host cells can for example be found in Sambrook et al. (Molecular Cloning: A Laboratory Manual, 2nd Edition, Cold Spring Harbor Laboratory press (1989)), and other laboratory manuals. “Transcriptional regulatory sequence/element” is a generic term that refers to DNA sequences, such as initiation and termination signals, enhancers, and promoters, splicing signals, polyadenylation signals which induce or control transcription of protein coding sequences with which they are operably linked. A first nucleic acid sequence is “operably-linked” with a second nucleic acid sequence when the first nucleic acid sequence is placed in a functional relationship with the second nucleic acid sequence. For instance, a promoter is operably-linked to a coding sequence if the promoter affects the transcription or expression of the coding sequences. Generally, operably-linked DNA sequences are contiguous and, where necessary to join two protein coding regions, in reading frame. However, since for example enhancers generally function when separated from the promoters by several kilobases and intronic sequences may be of variable lengths, some polynucleotide elements may be operably-linked but not contiguous.

Host Cells: In another aspect, the present disclosure provides a cell comprising or expressing the biosensors embodied herein. In an embodiment, the cell has been transfected or transformed with a nucleic acid encoding the above-defined first and/or second component(s). The disclosure further provides a recombinant expression system, vectors and cells, such as those described above, for the expression of the biosensors, using for example culture media and reagents well known in the art. The cell may be any cell capable of expressing the first and second component(s) defined above. Suitable host cells and methods for expression of proteins are well known in the art. Any cell capable of expressing the component(s) defined above may be used. For example, eukaryotic host cells such as mammalian cells may be used (e.g., rodent cells such as mouse, rat and hamster cell lines, human cells/cell lines). In another embodiment, the above-mentioned cell is a human cell line, for example an embryonic kidney cell line (e.g., HEK293 or HEK293T cells).

### Methods of Use

In certain embodiments, biosensor embodied herein is used in an assay, such as for example a high-throughput screening assays for assessing or identifying modulators of kinases. Accordingly, in certain embodiments, a method of identifying modulators of kinases comprises contacting a biosensor or a cell expressing a biosensor, with one or more candidate agents, wherein the biosensor comprises an enzyme substrate, a detectably labelled protein, and a phospho-amino acid binding protein; assaying for the presence or absence of a signal from the detectable label; and, identifying modulators of kinases. In certain embodiments, a signal from the detectable label is identified as an activator of the kinase. In certain embodiments, the absence of a signal from the detectable label is identified as an inhibitor of the kinase. In certain embodiments, the kinase is calcium/calmodulin-dependent protein kinase II (CaMKII). In certain embodiments, the CaMKII comprises an amino acid sequence having at least about sequence identity to MHRQETVDCLK (SEQ ID NO: 1) or SEQ ID NO: 2.

### Kits

The present disclosure provides for kits. As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. As used herein, the term “fragmented kit” refers to a delivery systems comprising two or more separate containers that each contain a subportion of the total kit components. The containers may be delivered to the intended recipient together or separately. For example, a first container may contain an enzyme for use in an assay, while a second container contains oligonucleotides. The term “fragmented kit” is intended to encompass kits containing Analyte specific reagents (ASR's) regulated under section 520(e) of the Federal Food, Drug, and Cosmetic Act, but are not limited thereto. Indeed, any delivery system comprising two or more separate containers that each contains a subportion of the total kit components are included in the term “fragmented kit.” In contrast, a “combined kit” refers to a delivery system containing all of the components of a reaction assay in a single container (e.g., in a single box housing each of the desired components). The term “kit” includes both fragmented and combined kits.

Accordingly, in certain embodiments, a kit comprises a synthetic biosensor embodied herein. In certain embodiments, a kit comprises an expression vector encoding a synthetic biosensor embodied herein. In certain embodiments, a kit comprises a host cell comprising a synthetic biosensor embodied herein or vector encoding a synthetic biosensor embodied herein. In certain embodiments, a kit comprises the calcium/calmodulin-dependent protein kinase II (CaMKII) substrate comprising SEQ ID NOS: 1, 2 or both.

### EXAMPLES

### Example 1 Novel CaMKII Biosensor Enables Identification of Potent CaMKII Inhibitors

Ca²⁺/Calmodulin-dependent protein kinase II (CaMKII) is a highly validated cause or contributor to major cardiac illnesses, including heart failure, myocardial infarction, and arrhythmias. Excessive CaMKII activity causes intracellular Ca²⁺ dysregulation, inflammation, maladaptive transcription, and cell death. Yet, there are currently no approved CaMKII inhibiting therapies. Thus, development of safe and effective CaMKII inhibitors is a translational priority.

Identification of potent CaMKII inhibitors has been hampered by a lack of a CaMKII reporter compatible with high throughput, live cell measurements. To address this, a novel genetically-encoded fluorescent CaMKII biosensor (CaMKAR:CaMKII Activity Reporter) was engineered. CaMKAR provides an ultrasensitive dynamic range of 364±0.029%, fast reporting kinetics with a half-time of ˜5.1 sec, and can operate in both intensiometric and ratiometric modes. Unlike previous biosensors, CaMKAR acts as a substrate for CaMKII and thus reports bonafide phosphorylation events. It was shown that CaMKAR is specific to CaMKII when tested against a panel of predicted off-target kinases (PKA, PKC, PKD, CaMKI, CaMKIV). Moreover, purified recombinant CaMKAR was used to develop an in vitro kinase assay that can measure active CaMKII present in cell and tissue lysates or with cell-free purified protein. Most importantly, the easy-to-measure CaMKAR fluorescent signal allows for unprecedented high throughput measurements in live cells via plate reader and flow cytometry.

As a proof of principle, CaMKAR was used to screen CaMKII inhibitor small molecules. A library of currently approved drugs was used, in part, because concerns for CaMKII inhibition as a therapeutic strategy relate to potential adverse consequences of CaMKII blockade on learning and memory. It was reasoned that if CaMKII inhibitors were successfully identified from drugs in common use, it would dispel these concerns. The Johns Hopkins Drug Library of 4,475 clinically-approved (FDA, EMA, CFDA, PMDA) compounds was screened on cells expressing constitutively active CaMKII. Primary hits were further screened in vitro to differentiate bonafide CaMKII inhibitors from autofluorescent compounds and phosphatase activators. This yielded 39 novel CaMKII inhibitors. Surprisingly, the majority of these compounds are not known to be kinase inhibitors.

### Example 2: Novel Biosensor Identifies Ruxolitinib as a Potent and Cardioprotective

### CaMKII Inhibitor

Cardiovascular diseases are leading causes of premature death, with over half a billion patients affected worldwide (1). The multifunctional Ca²⁺/calmodulin-dependent protein kinase II (CaMKII) contributes to physiological regulation of Ca²⁺ cycling in cardiomyocytes, but is surprisingly dispensable for cardiac function (2-4). In contrast, excessive CaMKII activity is cardiotoxic and CaMKII inhibition is protective in numerous models of inherited and acquired heart diseases and arrhythmias (5-13). CaMKII hyperactivity is, in part, a consequence of catecholamine stimulation but clinically approved drugs that inhibit β-adrenergic receptors ('s blockers') are ineffective at preventing increased CaMKII activity in myocardium obtained from heart failure patients (14). Thus, developing safe and effective direct CaMKII inhibitor drugs is a major unmet translational goal.

Although several inhibitory modalities have been described, none have reached the clinic due to various limitations. KN-62 and KN-93 were the first CaMKII inhibitors (15, 16). These allosteric inhibitors alleviate CaMKII-toxicity in animal models (7, 11, 17). While these remain popular as tool compounds, they were clinically hampered by low potency (IC₅₀>2 μM), prohibitive off-target toxicities, and most importantly, they fail to inhibit autonomously hyperactive CaMKII (18-20). In fact, KN-93 has been recently shown to be a calmodulin inhibitor, which explains its inability to inhibit active CaMKII (21). CaMKII inhibitory peptides (e.g., CaMKII-IN and AIP) have served as powerful genetic tools with impressive affinity and specificity (22, 23), but have failed to translate due to the challenges of peptide delivery: short plasma half-life, cell impermeability, and suboptimal viral delivery to human myocardium (24). While peptides can be modified to enhance cell penetrance and have reached clinical trials for other indications, these modifications have not yielded clinically applicable CaMKII inhibition. Aiming to address these limitations, several ATP-competitor small molecules have been identified or developed (25-29). From these, only the natural derivative DiOHF has made it to a human trial (30); this study intended to inhibit CaMKII after ischemia/reperfusion injury but yielded negative results-thought to be due to low potency against CaMKII (31). Since CaMKII is important for long-term potentiation and is abundant in excitatory synapses (32), concern for adverse effects on learning and memory has been a major obstacle to developing CaMKII inhibitors (24). It was reasoned that discovery of an approved medication, preferably in broad use, with potent CaMKII inhibitory properties would provide critical insights to inform translational researchers, patients, and industry about the viability of CaMKII inhibitors for patients.

Development of small molecule inhibitors could be facilitated by a biosensor with high throughput capability, but existing CaMKII sensors are not suitable for this. A seminal sensor, Camui, suffers from low dynamic range and reports on conformational changes rather than enzymatic activity (33, 34). A more recent sensor, FRESCA, elegantly bypassed this obstacle by sensing CaMKII activity via direct substrate phosphorylation of the sensor (35). This improvement over Camui is limited by FRESCA's dynamic range which is even lower than Camui (˜2.7%). Based on these limitations, it was sought to develop a CaMKII activity reporter that features both high sensitivity and direct kinase sensing-unlike FRESCA or Camui, which possess only one-thereby making it tractable for in cellulo screening.

In the present study, the two major gaps outlined above were address. First, a new genetically encoded CaMKII activity biosensor was engineered for live cell screening featuring the highest sensitivity and kinetics reported for a CaMKII sensor. Then, the safe-for-human pharmacopeia (4,475 compounds) was screened and several compounds capable of inhibiting hyperactive CaMKII were identified. From these, ruxolitinib displayed outstanding repurposing characteristics, including high potency at human dose equivalents, low toxicity, and known low brain penetrance. Ruxolitinib prevented inherited and acquired arrhythmias arising from CaMKII hyperactivity: catecholaminergic polymorphic ventricular tachycardia (CPVT) and atrial fibrillation, in validated murine disease models without impairing cognitive function.

### Materials and Methods

### Study Design

The goal of this study was to engineer a CaMKII biosensor suitable for high throughput screening and identify drugs that can be repurposed as CaMKII inhibitors. All animal studies were approved by appropriate animal care and use committees. Mice of both sexes were used unless noted in the methods. Animal number was determined by prior experience and published reports. Mice were randomly assigned to treatment conditions, and when an experiment spanned across multiple days, all conditions were represented in each cohort. Behavioral tests were performed by an independent experimenter blinded to the conditions. Experiments determining protection of ruxolitinib in CPVT models (cells and mice) were performed by experimenters blinded to the identity of the compound. Imaging data analysis was done in a computer-automated fashion to avoid human bias.

### Plasmids and Molecular Biology

To create CaMKAR, cpGFP and FHA1 domains were amplified from pcDNA3.1(+)-ExRai-AKAR2 (gift from Jin Zhang, Addgene plasmid #161753) into pcDNA3.1 and pET-6×His/TEV plasmid backbones. CaMKII substrates were added to the 5′ end of cpGFP by site-directed mutagenesis (KDL Enzyme Mix, NEB). deadCaMKAR^{T6A} was generated by site-directed mutagenesis of pcDNA3.1-CMV-CaMKAR. For lentiviral delivery of CaMKAR and CaMKIIδ, CaMKAR was cloned into pLV-hEF1a plasmid backbone to create pLV:hEF1a-CaMKAR-P2A-BlastR (VectorBuilder); CaMKIIδ was cloned into pLVX:TetONE-Puro-hAXL (gift from Kenneth Pienta, Addgene plasmid #124797). For neuronal expression, CaMKAR was subcloned into pCAGGS. PKC sensor pcDNA3.1-ExRai-CKAR was a gift from Jin Zhang (Addgene plasmid #118409). Constitutively active CaMKI^{T17D} was synthesized by Twist Bioscience. Constitutively active CaMKIV-dCT was a gift from Douglas Black (Addgene plasmid #126422). pcDNA3-Camui-NR3 was a gift from Dr. Michael Lin.

### CaMKAR-Based High Throughput Screening

A polyclonal K562^{CaMKAR-CaMKII} line was created by infecting 3M cells with lentivirus containing pLVX:EF1a-CaMKAR-BlastR (multiplicity of infection=5) and pLVX:TetONE-CaMKII^{T287D}-P2A-mCherry (multiplicity of infection=1). These cells were expanded to 600M and incubated with 1 μg/mL doxycycline 24 hours prior to screening. Cells were resuspended in Live Imaging Cell Solution (Gibco) supplemented with doxycycline and 4.5 g/L glucose. These cells were then distributed across 15 clear-bottom 384 well plates at 50 k cells per well. 5 μL of 10× compound from the Johns Hopkins Drug Library v3.0 was added at a final concentration of 5 μM per compound. The Johns Hopkins Drug Library v3 was assembled by combining the Selleckchem FDA-Approved Drug Library (3,174 compounds, Catalog No: L1300) with non-duplicate compounds in APExBIO DiscoveryProbe FDA-approved Library (483 compounds, Catalog No. L1021) and the MicroSource US Drug Collection (817 compounds). The library was formatted for use in 384-well plates and stored in DMSO at −80° C. Each plate contained 2 columns of untreated cells, and 1 column of cells treated with AS100397 10 μM. Cells were incubated for 12 hours before reading by high content imager (MolDev IXM High Content Imager). Images were automatically segmented and quantified using CellProfiler as above. CaMKII inhibitory % was calculated by min-max normalizing the data between untreated and AS100397-treated samples. For secondary screen, see supplementary methods.

### Mouse Models

All mouse studies were carried out in accordance with guidelines and approval of the Johns Hopkins University Animal Care and Use Committee (Protocol #M020M274) and Boston Children's Hospital (Protocol 20-05-4139). 8-12 week old Male C57BL/6J mice (The Jackson Laboratory, ME, USA) were housed in a facility with 12 hour light/12 hour dark cycle at 22±1° C. and 40 t 10% humidity. Teklad global 18% protein rodent diet and tap water were provided ad lithium.

### Cell Culture

HEK293T/17 cells (ATCC CRL-11268), K562 cells (ATCC CCL-243), and NRVMs were maintained in DMEM (L-glutamine, Sodium Pyruvate, Non-essential amino acids; Gibco) supplemented with 10% FBS (Gibco) and Pen/Strep (Gibco). Cells were maintained between 10%-95% confluence (HEK293T) or 100 k-1M cells/mL (K562). NRVMs were isolated from Sprague-Dawley rats as previously described (87). Rat hippocampal neurons were cultured as previously described (38). All cells were maintained in a humidified incubator at 37 C with 5% CO2. To determine viability, cells were lysed in CellTiter-Glo 2.0 (Promega) reagent and read with a Synergy MX microplate reader.

Recombinant CaMKAR: Purified CaMKAR was generated by transforming NEBExpress Competent E. coli (NEB) with pET-6×his/TEV-CaMKAR. After addition of Isopropyl Thiogalactoside (IPTG, 0.4 mM) and incubation at 15° C. for 24 hours, cells were lysed and sonicated. CaMKAR was isolated from soluble lysate by nickel chromatography NEBExpress columns (NEB). Soluble protein was quantified by Pierce BCA assay (ThermoFisher) and stored in 50% glycerol at −80 C.

### Gene Transfer to Cultured Cells

Lentivirus production and infection: HEK293T/17 cells under passage #6 were cultured in 10 cm dishes at 400 k cells/mL. These cells were transfected using TransIT-Lenti (Mirus Bio) using a ratio of 5:3.75:1.25 of packaging plasmid:psPAX2:pMD2.G for a total of 10 μg per dish. After 48 hours, supernatant was collected, clarified, and concentrated 10-fold using Lenti-X concentrator (Takara). Lentivirus aliquots were stored at −80° C. until functional titration and use. Infection occurred at indicated multiplicity of infection in the presence of 10 μg/mL polybrene reagent (Sigma).

Plasmid transfection: HEK293T/17 cells were plated into well plates at 400 k cells/mL unto PDL coated 24 well plates. Each well was transfected with 500 ng of DNA complexed with 1 μL of JetPrime reagent. Cells were examined 24-48 hours post transfection. Rat neurons were transfected with the indicated plasmids using Lipofectamine 2000 (Invitrogen) at 13-18 days in vitro.

Adenovirus and siRNA in NRVMS: CMV-CaMKAR-encoding Ad5 adenovirus was synthesized by Vector Biolabs. NRVMs were infected 24 hours post isolation at multiplicity of infection 100. Anti-CaMKIIδ (s127546, Thermo Fisher) and scrambled siRNA (AM4611, Thermo Fisher) were transfected using 10 pmol complexed with Lipofectamine RNAiMAX (Thermo Fisher).

### Microscopy

Timelapse microscopy was performed using an Olympus IX-83 inverted widefield microscope equipped with an ORCA Flash 4.0 and Lumencor SOLA light source. CaMKAR signal was captured at 200 ms exposure using the following channels: excitation filters ET402/15x and ET490/20x and emission filter ET525/35m (Chroma Technology). CaMKAR Signal (R) is defined as the ˜488-nm-excited intensity divided by the ˜400-nm-excited intensity. Calcium imaging was collected in the TRITC channel ex 555/em. Confocal imaging was performed using a Zeiss LSM880 Airyscan FAST. Using excitation lasers 405 nm and 488 nm and collecting emission window at 520±10 nm. Otsu segmentation was used to track individual cells and their intensity in 488 nm and 405 nm channels at every timepoint in CellProfiler.

Rat neurons were imaged 3-7 days after transfection on a Zeiss spinning-disk confocal microscope. Dual excitation-ratio imaging was performed using the 488- and 405-nm lasers and a 525/50 emission filter. Image analysis was performed using custom scripts in Fiji/ImageJ.

Induced Pluripotent Stem Cells (iPSCs) Generation, iPSC-Cardiomyocyte Differentiation and Imaging

Patients with pathogenic RYR2 mutations manifesting catecholaminergic polymorphic ventricular tachycardia (CPVT) provided informed consent to participate in the study under protocols approved by the Boston Children's Hospital Institutional Review Board. Peripheral blood mononuclear cells were reprogrammed to pluripotency with the CytoTune Sendai reprogramming kit (ThermoFisher). iPSCs were differentiated to iPSC-CM and their purity was assessed by flow cytometry (13).

### Kinetic Image Cytometry

For adult cardiomyocytes: Isolated adult cardiomyocytes were stained with 8 μM Rhod-2AM for 15 minutes at 37 C, washed with Tyrode solution and incubated with DMSO (1:1000), experimental compounds (2 μM), isoproterenol (100 nM), or isoproterenol (100 nM) plus autocamtide-2-related inhibitory peptide (AIP) (3 μM) for calcium transient measurements after electrical stimulation (30 pulses) in a CyteSeer Scanner (Vala Sciences) at 8 Volts.

For iPSC-CMs: iPSC-CMs were stained with 5 μM Fluo-4 for 15 minutes at 37 C, washed with RPMI and incubated with DMSO (1:1000), experimental compound (2 μM), isoproterenol (1 μM), or isoproterenol (1 μM) plus autocamtide-2-related inhibitory peptide (AIP) (3 μM) for calcium transient measurements after electrical stimulation (10 pulses) in a CyteSeer Scanner (Vala Sciences) at 15 Volts. After CyteSeer data acquisition, offline data analysis was performed, where ImageJ was used for manual cell segmentation, and a custom MATLAB (Mathworks) script was used for calcium transient data filtration, parameter calculation, and post-pacing abnormal calcium release event analysis. Post-pacing abnormal calcium release events were defined by a peak amplitude >5% of the median pacing calcium transient amplitude for a given cell. Microsoft Excel, and GraphPad Prism 9.3 software were then used for data compilation, statistical analysis, and graphical representation. Imaging data obtained from adult cardiomyocytes or iPSC-CMs were analyzed using mixed ANOVA followed by Wald's chi-squared test (88).

### Secondary Drug Screen

118 candidates from in cellulo screen were then subjected in vitr CaMKAR screen. In vitro CaMKAR reaction was carried in 384-well plates with 25 μL total volume, 12.5 μL 2× buffered PBS, 1.25 μL 10 mM CaCl, 0.5 μL 50 μM CaM, 0.11 μL CaMKAR (25 μmol), 0.0625 μL CaMKII (125 fmol), completed with nuclease-free water. Plate was read at baseline via Tecan Safire microplate reader, then 5 μL of 5× drug was added (5 μM final), plate read again, then 5 μL ATP was added (100 μM final) and kinetic assay was read for 60 mins. Slope in the change of CaMKAR ratio was used as metric for CaMKII activity. Data was min-max normalized between untreated and AS100397-treated wells. Hits for both screens were defined by significant deviation from untreated distribution by the z-Statistic p-Value. This statistical approach was reviewed by the Johns Hopkins Biostatistics, Epidemiology, and Data Management Core.

### Cell-Free Kinase Activity Assays

In vitro CaMKIIδ assays were performed by Reaction Biology Corporation as follows: Substrate is prepared in freshly prepared Reaction Buffer (20 mM Hepes (pH 7.5), 10 mM MgCl₂, 1 mM EGTA, 0.01% Brij35, 0.02 mg/ml BSA, 0.1 mM Na₃VO₄, 2 mM DTT, 1% DMSO) along with CaMKII-specific co-factors. Kinase is delivered into the substrate solution. Compounds are delivered in 100% DMSO into the kinase reaction mixture by Acoustic technology (Echo550; nanoliter range) followed by incubation for 20 min at room temp. ³³P-ATP is delivered into the reaction mixture to initiate the reaction. Incubate for 2 hours at room temperature. Kinase activity is detected by P81 filter-binding. In vitro assays for confirmation of secondary chemical screening (Datafile S2) were performed by Eurofins DiscoverX Corporation using the scanELECT assay.

### Experimental Mouse Procedures

Isoproterenol challenge: ruxolitinib phosphate (MedChemExpress) was suspended in sterile saline with 10% DMSO. Drug was administered via intraperitoneal injection. 10 minutes later, 5 mg/kg isoproterenol (Sigma-Aldrich) in saline was injected via intraperitoneal injection. Mice were euthanized 10 minutes after the isoproterenol challenge.

Cardiomyocyte Isolation: Adult ventricular cardiomyocytes were isolated from wildtype or transgenic Ryr2^{R176Q/+} CPVT mice by retrograde perfusion of the aorta with enzymatic digestion (collagenase type 11, Worthington) in a Langendorff apparatus (13), and plated on laminin-coated 96-well glass-bottomed plate for calcium transient measurements initiated by electrical stimulation (8 Hz). Kinetic Image Cytometry (KIC) measurements were performed to the attached cardiomyocytes after staining with Rhod-2AM (15 minutes at 37° C.). The cells were constantly maintained in Tyrode solution containing 137 mM NaCl, 20 mM HEPES, 10 mM D-glucose, 5.4 mM KCl, 1.2 mM MgCl₂×6H₂O, 1.2 mM NaH₂PO₄×2H₂O, 10 mM Taurine, 10 mM BDM, 1 mM CaCl₂)×2H₂O, pH 7.5.

Diabetic Mouse Model. Type-1 diabetes (T1D) was induced as previously described (57) in adult male C57BL/6J mice (The Jackson Laboratory, ME, USA) by a single intraperitoneal injection of streptozotocin (STZ)(185 mg/kg, Sigma-Aldrich) dissolved in a citrate buffer (citric acid and sodium citrate, pH 4.0) after a six hour fast. Mice were maintained on normal chow diet (NCD) (7913 irradiated NIH-31 modified 6% mouse/rat diet—15 kg, Envigo, Indianapolis, TN). Mice were considered diabetic if blood glucose level was ≥300 mg/dl via tail vein blood checked with a glucometer—OneTouch Ultra 2 (LifeScan, Zug, Switzerland).

Electrophysiological studies in CPVT mice: Adult mice (aged 8-12 weeks old) were anesthetized and instrumented to a 2-lead (leads I and II) ECG with a 1.1 F octapolar catheter (ADInstruments) being inserted into the right ventricle via a right jugular vein. Dimethylsulfoxide (DMSO) (1:1000), ruxolitinib (75 mg/Kg), isoproterenol (2 mg/kg) and epinephrine (3 mg/kg) were administered intraperitoneally, while surface and intracardiac ECGs were recorded for 3 to 15 minutes post-injection. More than 3 PVCs or a single couplet during this recording period was considered a positive response. Programmed ventricular stimulation was performed using a digital stimulator (ADInstruments) that delivered 8 stimuli (S1 8 times) followed by 2 early extra-stimuli (S2 and S3). Ventricular arrhythmias were defined at ≥3 induced ventricular beats. The duration of arrhythmias was measured from the last paced beat (13). QT dispersion was measured by comparing the absolute difference in the corrected QT interval between ECG leads I and II, while the heart rate-corrected QT (QTc) interval was calculated with an automated algorithm in LabChart (13).

Atrial fibrillation induction: Anesthetized mice were injected via intraperitoneal route with either ruxolitinib phosphate (MedChemExpress)—75 mg/kg dissolved in sterile saline with 10% dimethylsulfoxide (DMSO) and 2.5% tween-20—or vehicle 10 minutes prior to rapid atrial burst pacing to assess atrial fibrillation (AF) inducibility. 75 mg/kg dosing was chosen due to previous studies determining this dose to be equivalent to the 20-25 mg dose in humans based on plasma concentration (47-49). In vivo electrophysiology (EP) studies were performed as previously reported (57) in mice anesthetized with isoflurane (2% for induction and 1.5% for maintenance of anesthesia; Isotec 100 Series Isoflurane Vaporizer; Harvard Apparatus). AF was defined as the occurrence of rapid and fragmented atrial electrograms with irregular AV-nodal conduction and ventricular rhythm for at least 1 second. If 1 or more atrial bursts (out of 5) were an AF episode, the mouse was considered to have inducible AF. Mice were euthanized immediately following the procedure and atrial tissue from both right and left atria were obtained and flash frozen in liquid nitrogen and then stored at −80° C.

Spontaneous AF. 8-months-old CREM-IbAC-X mice were monitored via surface ECG for 15 minutes to establish the baseline for time spent in atrial fibrillation (AF). AF was defined as loss of p-waves and irregularly irregular R-R intervals for >10 seconds. Each mouse was monitored at least twice with more than 24 hours interval between two recordings. After establishing the baseline, the mice were injected with placebo (10% DMSO, 2.5% Tween-20 in saline; i.p. injections) and the 15-minute ECGs were recorded between 10-25 minutes post injection. Ruxolitinib injections (75 mg/kg in 10% DMSO, 2.5% Tween-20 in saline; i.p. injections) were performed with more than 24 hours intervals and the ECGs were recorded between 10-25 minutes post injection.

### Behavioral Paradigms

Memory tests were performed at the JHU Behavioral Core by a blinded experimentalist on male mice. Short term memory was assessed using the novel object recognition test. The test consisted of three phases over two days. On day 1 mice were habituated to the arena for 10 minutes. On day 2, mice were allowed to explore the arena consisting of two identical objects, referred to as the familiar objects, for 10 minutes and were then placed back in the home cage for 30 minutes. Following the delay, the mice were placed back into the arena with one “familiar” object and one “novel” object and allowed to explore the objects for 5 minutes. Distance travelled and time spent investigating each object was automatically recorded using Anymaze tracking software (Stoelting Co., Wood Dale, IL, USA).

Spatial memory was tested via the Y-maze spatial recognition test. The Y-maze consists of three 38 cm-long arms (San Diego Instruments). During the training phase, one arm of the Y-maze was blocked. The mouse was placed at the end of one of the two open arms and allowed to explore for 5 min. After a 30-min inter-trial interval, the test phase began: the blockade was removed, and the mouse was allowed to explore all three arms of the maze for 5 min. Distance traveled and time spent in each arm was automatically recorded using Anymaze tracking software. Data from the first 2 min of the test phase were used to evaluate percent time spent in the novel arm.

For the Barnes maze: A brightly lit (1100 lux) Barnes maze with 20 evenly spaced holes and an escape box placed under one of the holes was used (Maze Engineers). During training, each mouse was placed in the center of the maze and allowed to explore the maze for 3 minutes per trial. During the trial, the number of head dips and the latency to find and then enter the escape box were recorded. Mice were given two trials per day for four days. 24 hours following training, mice were given a probe trial, with the number of head dips and latency to find and then enter the escape box recorded.

### Western Blotting

Mouse hearts were disrupted using a tissue blender in 1% Triton X-100 containing protease and phosphatase inhibitors. To detect phospho- and total phospholamban, samples were left unboiled to preserve pentameric form. Samples were run on 4-12% bis-tris acrylamide gels and transferred onto nitrocellulose membranes. Total and phosphorylated-T17 phospholamban were assayed by incubation with respective antibodies (Phospholamban pT17 pAb (Badrilla A010-13), 1:5000; Phospholamban (Thermofisher/Pierce MA3-922), 1:2000) followed by secondary labeling (Goat Anti-Rabbit IgG Antibody 680LT (Licor 926-68021) 1:10,000; Goat Anti Mouse IgG Antibody 800CW (Licor 926-32210) 1:10,000) and quantified using a LICOR Odyssey imager and ImageStudio.

For RyR2^{R176Q/WT} animals: Heart tissues were shock frozen and homogenized in a buffer (˜500 μL) containing: 120 mM NaCl, 1 mM EDTA, 10 mM Glycerophosphate, 40 mM HEPES, 40 mM NaF, 0.3% CHAPS, 1% Triton X-100 and 1% HALT, pH 7.5. Proteins were quantified using Pierce BCA protein assay (Thermo Fischer Scientific). Samples were boiled at 95-C for 5 min and 10-15 μg of total protein were loaded per lane and subjected to 15% SDS-PAGE and immunoblot analysis. For uncalibrated optical density, all scanned blots were analyzed by ImageJ software.

### Schematics

Schematics and drawings made with Biorender.com. CaMKAR Ratio pseudocoloring for fluorescence microscopy performed with ImageJ using the Ratio Plus plugin. Kinase dendrogram illustration made with KinMap (89) and reproduced courtesy of Cell Signaling Technology, Inc. (cellsignal.com).

### Statistics

Imaging summary data was condensed and organized with R Studio. Statistical testing done with GraphPad Prism v8.2.0 as described in each figure. Signal-to-noise ratio was calculated as described (37): single-cell maximal ratio change after stimulation divided by the standard deviation across 4 baseline timepoints.

### Results

### Development of a CaMKII Biosensor Suitable for High Throughput Screening

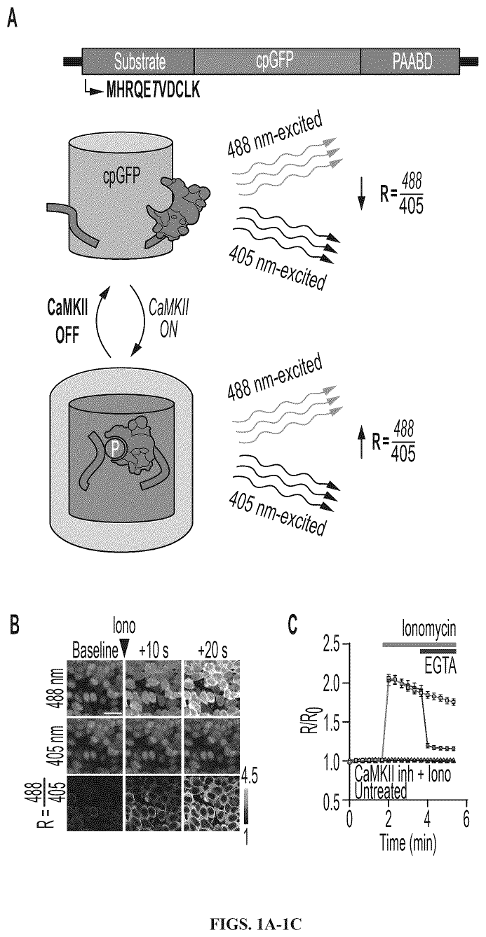

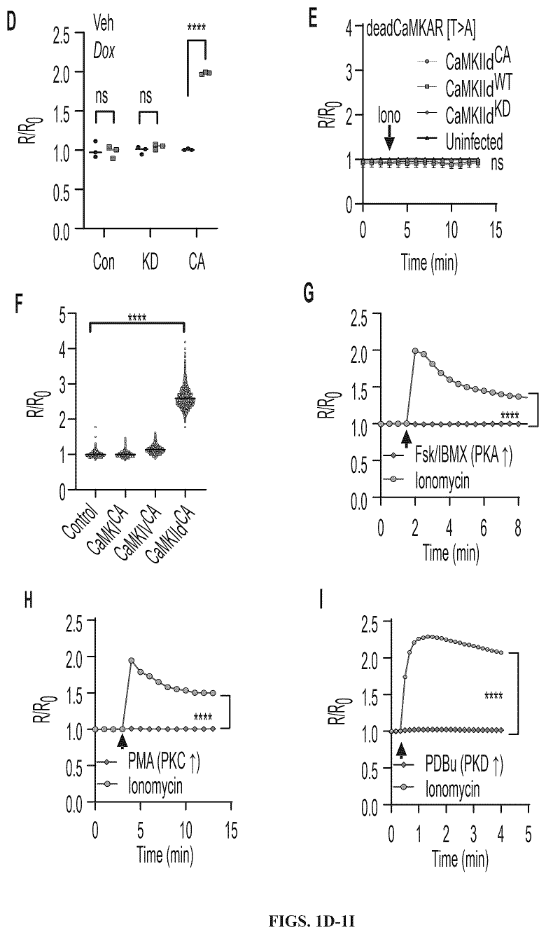

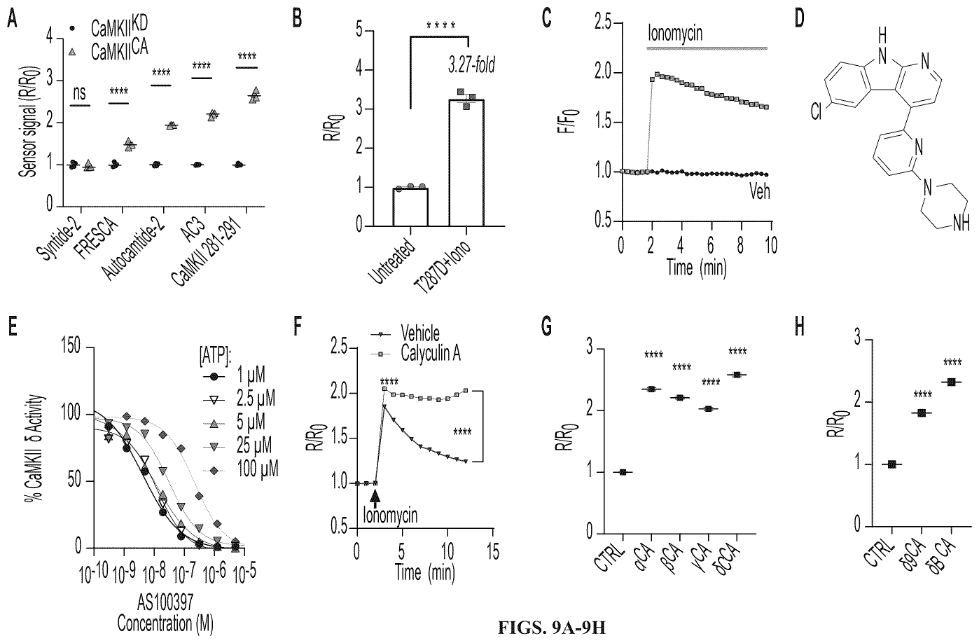

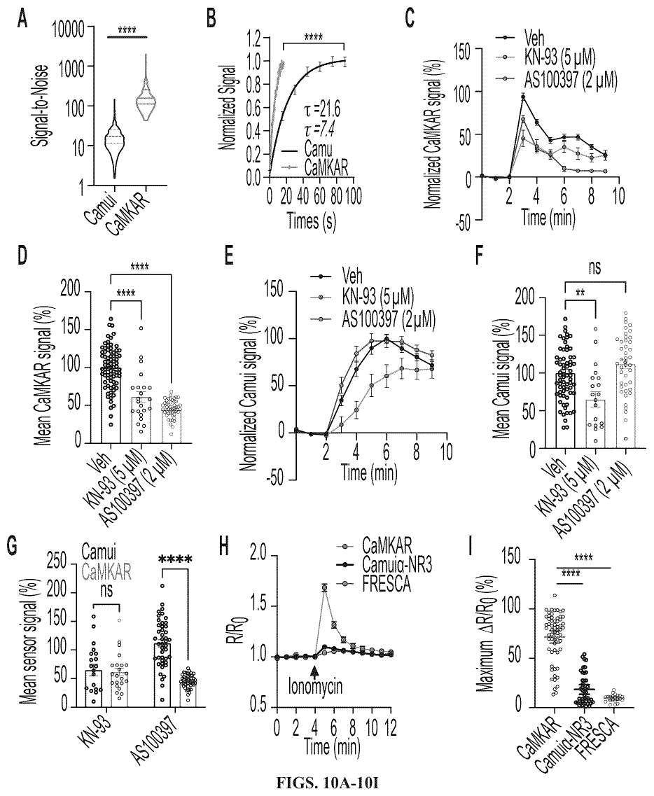

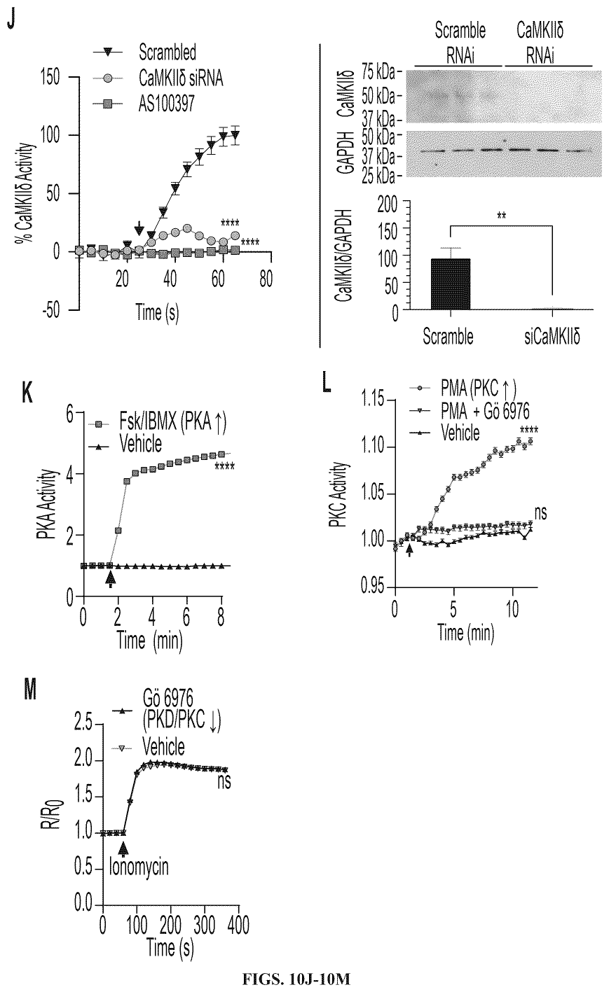

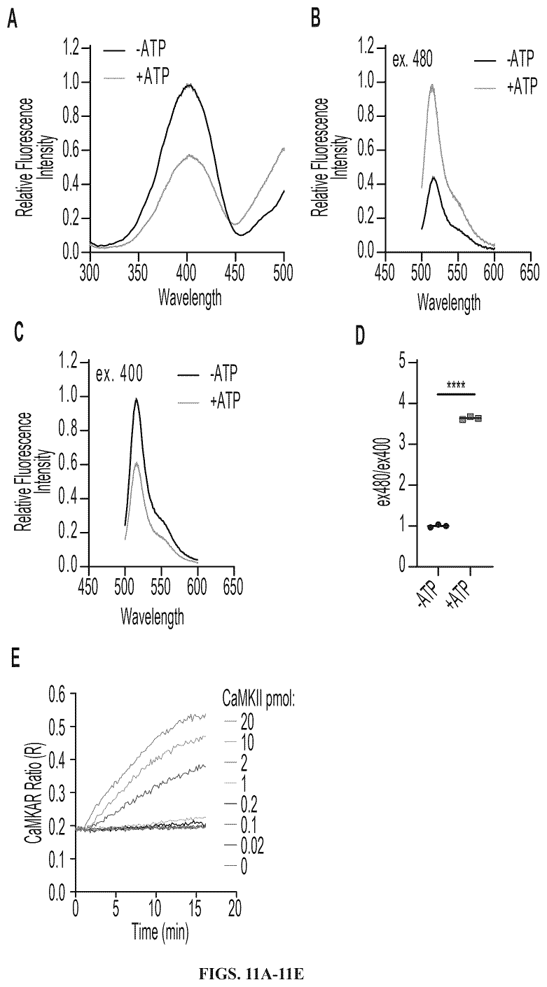

The discovery of circularly permuted green fluorescent protein (cpGFP) has led to numerous sensors that detect ions, metabolites, and enzyme activity by coupling reconstitution of GFP fluorescence with the process of interest (36-38). Using kinase-sensing cpGFP (38), a new sensor that reports on CaMKII activity was engineered. Screening among known CaMKII substrates, the CaMKII autophosphorylation peptide MHRQETVDCLK (amino acids 281-291 from human CAMKIIδ) fused to the 5′ end of kinase-sensing cpGFP led to the highest CaMKII-dependent response (FIG. 9A). This sensor was referred to as CaMKAR (CaMKII Activity Reporter), composed of the substrate peptide, cpGFP, and a phosphoresidue binding domain. Upon phosphorylation of the substrate by CaMKII, the phosphoresidue binding domain causes an intramolecular conformational change that restores GFP fluorescence (FIG. 1A). Akin to predecessor sensors, this reconstitution is excitation-ratiometric: GFP emission (˜520 nm light) excited by ˜488 nm light increases whereas GFP emission excited by ˜405 nm is unchanged or decreases (37, 38). CaMKAR signal was therefore expressed as the ratio (R) of these two channels (FIG. 1A). To test CaMKAR dynamics, CaMKAR-expressing HEK293T cells were treated with ionomycin, causing intracellular Ca²⁺ influx and activation of endogenous CaMKII (FIGS. 1B and 1C). This resulted in a rapid increase in R relative to baseline (R/R₀). Stimulation with ionomycin in HEK293T cells expressing constitutively active CaMKII revealed an in cellulo maximal dynamic range of 3.27 fold, or 227±11.1% (FIG. 9B). Since the 405 nm channel is largely unchanged upon stimulation, CaMKAR can also be used intensiometrically by only quantifying the 488 nm channel intensity; this modality retains 96.4±1.6% of the dynamic range compared to ratiometric mode (FIG. 9C). Pretreatment with the tool CaMKII-inhibitor AS100397 (FIGS. 9D and 9E)(39, 40) eliminates and post-treatment with the Ca²*-chelator EGTA rapidly reduces CaMKAR signal (FIG. 1C). It was validated that CaMKAR senses bona fide CaMKII-catalyzed phosphorylation by 3 orthogonal approaches: 1) expression of constitutively active CaMKIIT2D, but not kinase-defective CaMKII^{K43M}, increases CaMKAR signal (FIG. 1D); 2) CaMKAR sensing to genetic and pharmacological activation of CaMKII is completely abolished when the substrate threonine residue is mutated to alanine (FIG. 1E); and 3) CaMKAR signal is enhanced and fails to resolve when co-incubated with the phosphatase inhibitor Calyculin A (FIG. 9F). Lastly, CaMKAR is sensitive to all 4 human CaMKII isoforms (α, β, γ, δ) and the 3 prevalent splice variants of CaMKIIδ (δC, δB, δ9) (FIGS. 9G and 9H).

Next, it was sought to benchmark CaMKAR's performance and specificity. Camui was the first CaMKII biosensor developed and is still widely used (33, 34). However, it has important limitations: Camui contains and overexpresses brain isoform CaMKIIα, has relatively low dynamic range (˜5-70% versus CaMKAR's 227±11.1%), and, most notably, it reports conformational change rather than enzymatic activity (Table S1) (34, 41). In HEK293T cells, CaMKAR displays nearly 10-fold greater signal-to-noise (FIG. 10A) and is ˜3-fold faster (_{TCaMKAR}=7.4 vs _{TCamui}=21.6) (FIG. 10B). In cultured rat hippocampal neurons, Camui failed to detect catalytic inhibition of CaMKII, whereas CaMKAR detected both catalytic and allosteric inhibition (FIGS. 10C-10G). Head-to-head comparison in these cells demonstrated that CaMKAR is more sensitive than both Camui and FRESCA under the same stimulus (FIGS. 10H and 10I). Thus, CaMKAR enjoys improved sensitivity and kinetics. Since Ca²⁺ mobilizing agents can activate many kinases, it was validated that CaMKAR is not reporting on closely related/Ca²⁺-responsive kinases. Supporting specificity, CaMKAR signal was abrogated in cardiomyocytes treated with AS100397 and anti-CaMKIIδ siRNA (FIG. 10J). CaMKAR is insensitive to constitutively active CaMKI and CaMKIV (FIG. 1F). CaMKAR also failed to sense Forskolin/IBMX-mediated PKA activation, PMA-mediated PKC activation, and PDBu-mediated PKD activation (FIGS. 1G-II). It was verified that the concentrations of Forskolin/IBMX and PMA used in these studies were sufficient to elicit PKA (FIG. 10K) and PKC activity (FIG. 10L). Similarly, CaMKAR's response to ionomycin was unhindered by G66976, a dual PKC/PKD inhibitor (FIG. 10M).

Given its unimolecular architecture, it was sought to leverage CaMKAR into an in vitro assay. Recombinant CaMKAR is fully functional and exhibits appropriate spectral changes upon incubation with purified CaMKII (FIG. 11A). These changes are ATP-dependent, further confirming a dependence on phosphorylation for sensor function (FIGS. 11A-11C). CaMKAR displays a larger dynamic range in vitro (264±2.1%) compared to cell-based assays (227 f 11.1%), and the rate of the reaction is determined by the amount of CaMKII (FIGS. 11D and 11E). Altogether, it was demonstrated that CaMKAR is a bonafide CaMKII activity biosensor with unprecedented sensitivity, fast kinetics, specificity, and suitability for live cell and in vitro drug screening.

### CaMKAR-Based Screen of Drugs in Clinical Use

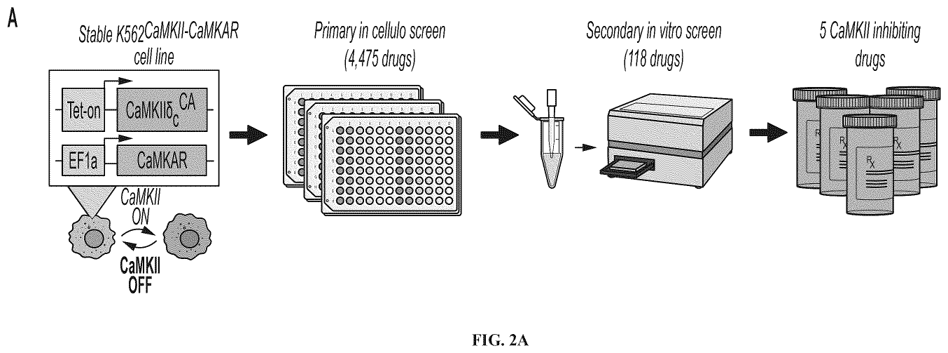

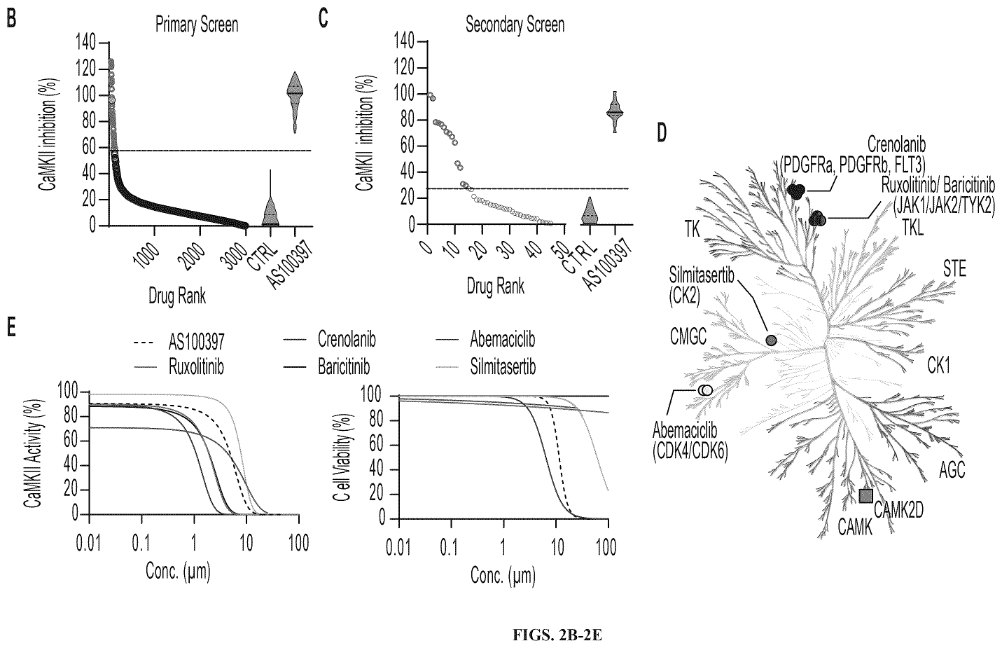

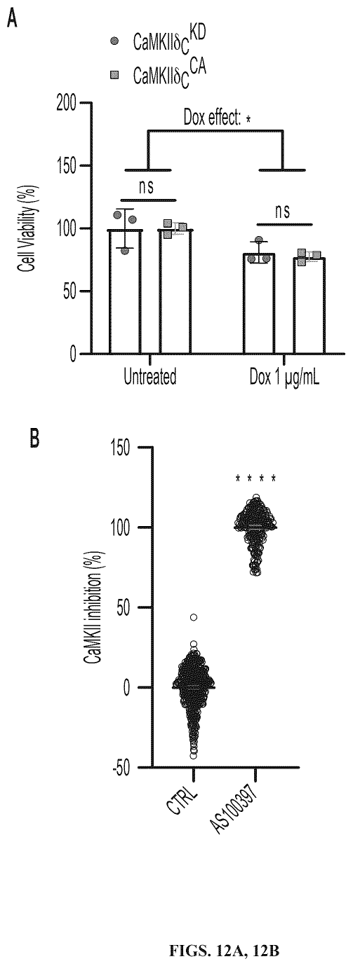

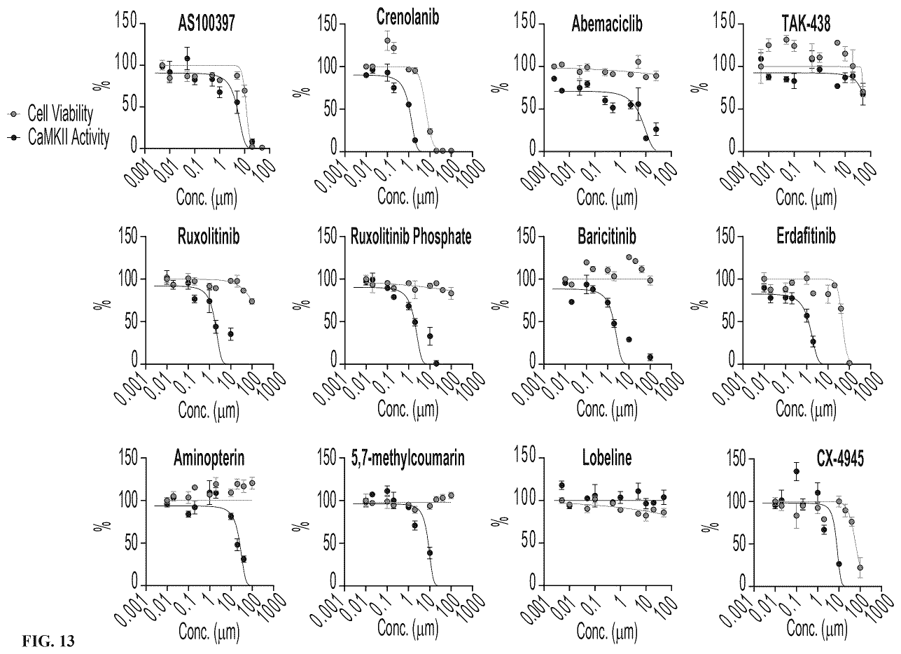

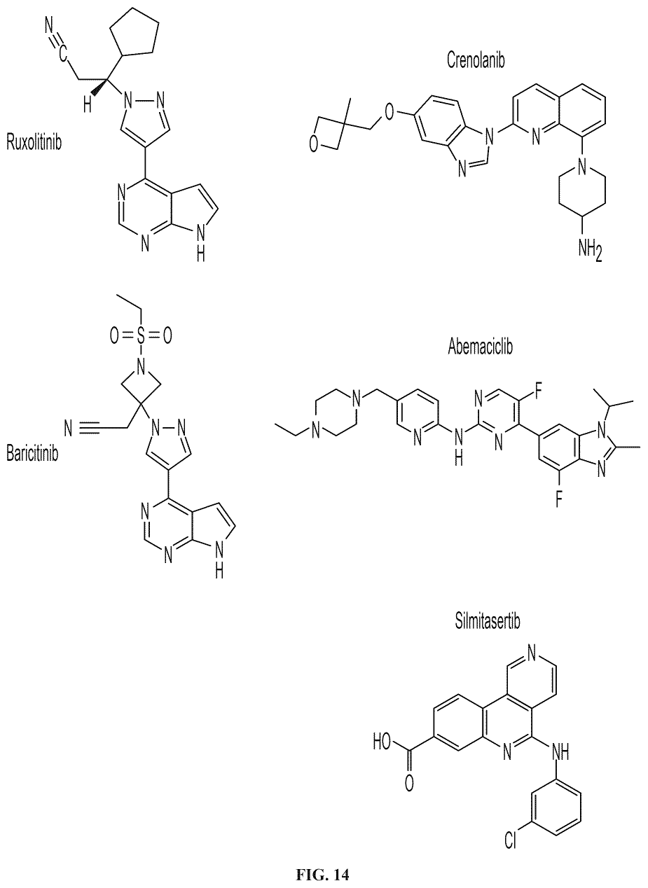

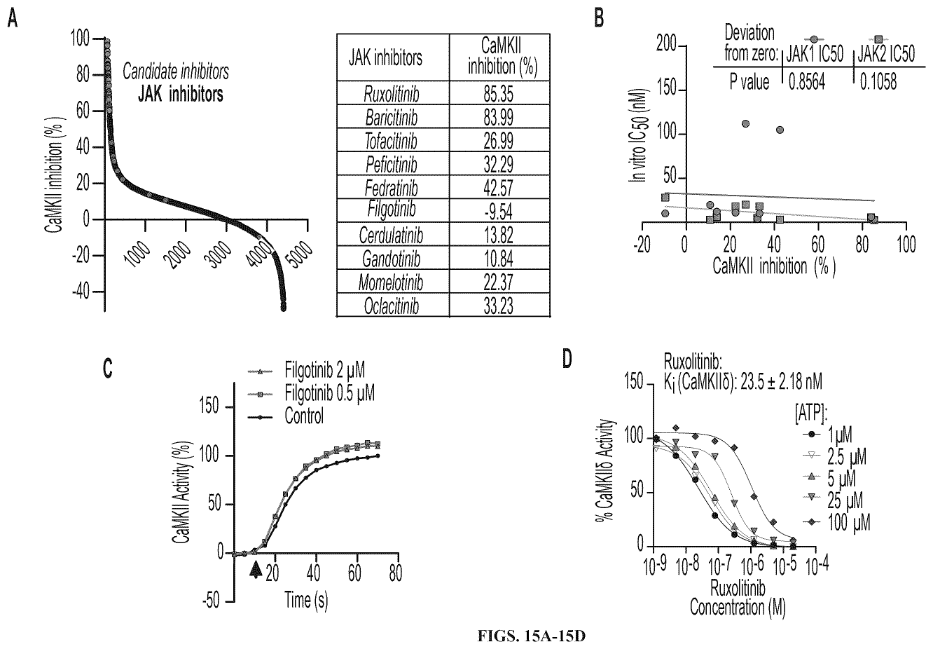

Next, the clinically-approved pharmacopeia was canvassed for drugs that are safe for human pharmacotherapy and potent CaMKII inhibitors. Due to its high throughput tractability, CaMKAR is uniquely configured to address this task. For screening, a stable line of human K562 cells were first created that co-express CaMKAR and CaMKIIδ^{CA}: K562^{CaMKII-CaMKAR} (FIG. 2A). K562 cells were chosen because they grow at high density and remain viable after expression of active CaMKII (FIG. 12A). The use of constitutively active CaMKII instead of Ca²⁺/calmodulin-activated CaMKII increases the likelihood of identifying catalytic inhibitors rather than calmodulin inhibitors (such as KN-93). The sensitivity of CaMKAR enables screening to occur in small culture volumes: CaMKII inhibition by AS100397 is detectable in as little as 30 μL of cell culture (FIG. 12B). As a primary screen, this cell line was tested against the Johns Hopkins Drug Library v3.0—constructed by pooling 4,475 compounds approved for human use by regulatory agencies from the U.S., Europe, Japan, and China. This library targets 44 different pathway families and contains over 100 kinase inhibitors. After 12 hours of treatment, cells were assayed for CaMKAR signal using high content imaging. Among drugs with an inhibitory signal, 118 compounds were found that reduced CaMKII activity by 60% or more, representing a false discovery-adjusted p value cutoff of <3×10⁻⁵ (FIG. 2B). It was reasoned that these hits likely contained a mixture of genuine inhibitors and false positives (e.g., auto-fluorescent compounds, indirect inhibitors, and phosphatase activators). To discern true inhibitors from false positives, a secondary screen was performed using the CaMKAR in vitro assay. In this assay, fluorescence was measured in a mixture of CaMKII, Ca²⁺-bound calmodulin (Ca²⁺/CaM), and CaMKAR at baseline, after drug addition, and after catalysis was initiated with ATP. Thus, this screen simultaneously determined whether a drug is auto-fluorescent and/or a direct inhibitor. This revealed 13 compounds with statistically significant inhibition (FIG. 2C). Importantly, both screening steps returned the positive control, pan-kinase inhibitor staurosporine. To remove weak inhibitors and further validate the results, these compounds were re-tested in HEK293T cells (FIG. 13) and in vitro by an independent commercial laboratory. This confirmed 5 potent CaMKII inhibitors: ruxolitinib, crenolanib, baricitinib, abemaciclib, and silmitasertib; all known to be ATP-competitive kinase inhibitors (FIGS. 2D, 14). Remarkably, none of the compounds were designed against kinases in the CAMK superfamily, and 3 out of the 5 compounds are intended against tyrosine kinases-one of the most dissimilar kinase families to CAMK by kinase domain homology (FIG. 2D). Unlike KN-93 (23), all 5 compounds inhibited Ca²⁺-independent, autonomously hyperactive CaMKII in HEK293T cells (FIG. 2E, left panel). In this assay, crenolanib, ruxolitinib, and baricitinib were more potent than AS100397, and all drugs except crenolanib were less toxic after a 12-hour exposure (FIG. 2E, right panel). Thus, ruxolitinib and baricitinib stand out as being more potent and less toxic than the tool compound in cultured cells.

### Clinically Approved Drugs Inhibit CaMKII in Cardiac Cells

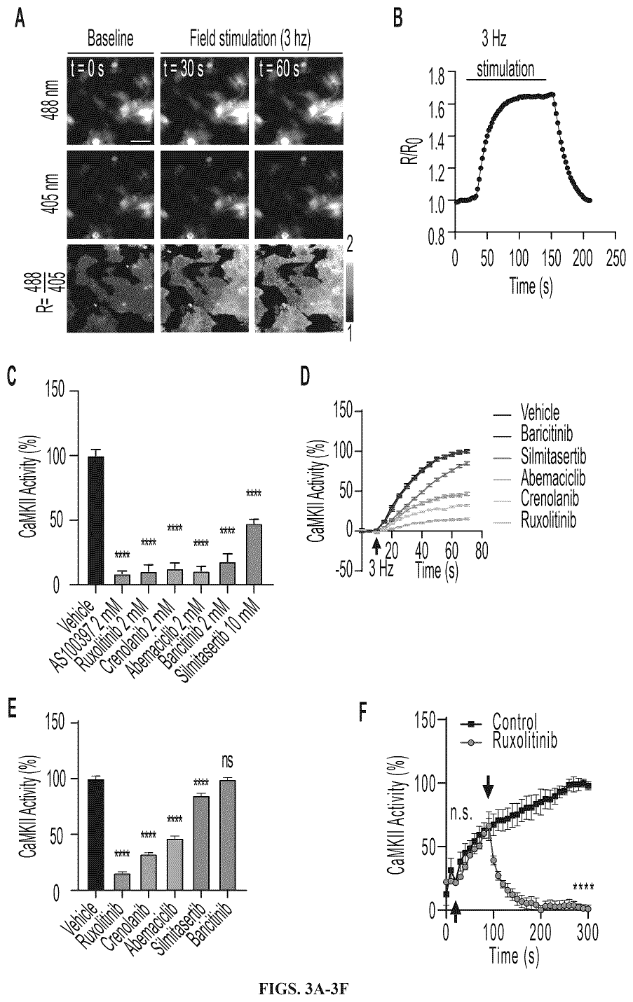

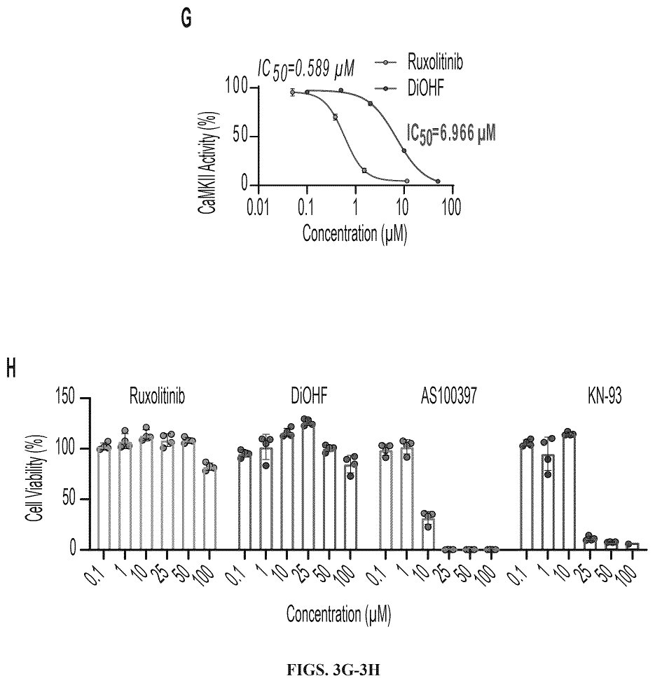

Because of the established benefits of CaMKII inhibition in cardiovascular disease models, it was queried whether these 5 compounds could inhibit CaMKII in cardiomyocytes. In neonatal rat ventricular myocytes (NRVMs), CaMKII activity can be stimulated by rapid field pacing (FIGS. 3A and 3B). All five compounds inhibited this response at high doses (FIG. 3C). However, to determine which compounds are likely to have an effect at clinically achievable doses, each concentration was adjusted to their respective maximal human plasma concentration (42-46). This revealed that ruxolitinib, crenolanib, abemaciclib, and silmitasertib, but not baricitinib, sustained inhibition (FIGS. 3D and 3E). Due to its high potency, low toxicity, and favorable safety profile among the hits (Table S2), ruxolitinib was henceforth focused on. It was first ascertained that ruxolitinib is capable of inhibiting pre-activated CaMKII, returning activity levels to baseline within 30 seconds in cardiomyocytes under continuous pacing (FIG. 3F). It was found that ruxolitinib is over 10-fold more potent than 3′,4′-dihydroxyflavonol (DiOHF) (FIG. 3G) (29, 30). This inhibitory effect appears to be independent of ruxolitinib's JAK1/2 inhibition. For one, multiple JAK1/2 inhibitors were found in the screen that failed to reduce CaMKAR signal (FIG. 15A). Among these compounds, their known IC₅₀ values against JAK1/2 did not significantly correlate with CaMKAR inhibition in our screen (FIG. 15B). Filgotinib, another FDA-approved drug with similar potency against JAK1/2 failed to inhibit pacing-induced CaMKII activity (FIG. 15C). Lastly, an in vitro biochemical assay confirmed inhibition of CaMKII in the absence of JAK1/2; this revealed that ruxolitinib has an inhibitory constant (Kᵢ) of 23.4 nM±2.18 (compared to DiOHF which has a published IC₅₀ of 250 nM) and verified an ATP-competitive mechanism (FIG. 15D). 48-hour exposure in NRVMs revealed that ruxolitinib is well tolerated up to 100 μM. This was similar to DiOHF and vastly superior to KN-93 and AS100397 (FIG. 3H). It was concluded that the drugs identified in the screen share the ability to inhibit CaMKII in cardiac cells. But among these, ruxolitinib appears to be best suited for repurposing due to its potency relative to its human dose and low toxicity.

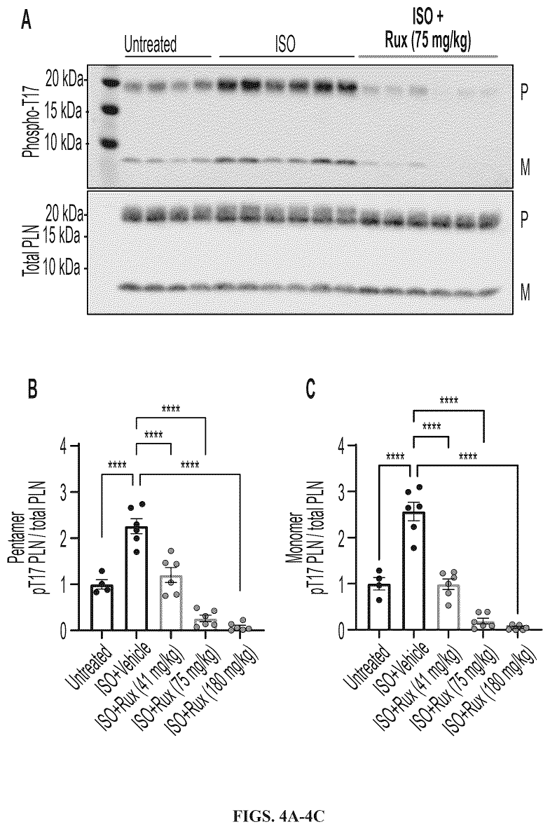

It was then evaluated whether ruxolitinib could replicate its CaMKII inhibition in vivo. 60-90 mg/kg dosing in mice is considered equivalent to the prescribed 20-25 mg doses in humans based on equivalent resulting plasma concentrations (47-49). 10 minute systemic pre-treatment with ruxolitinib at 41, 75, and 180 mg/kg suppressed isoproterenol-induced phosphorylation of phospholamban at threonine 17—a validated marker of CaMKII activity (50-52)—in a concentration-dependent manner (FIGS. 4A-4C). Altogether, the data demonstrate that ruxolitinib inhibits CaMKII in cardiomyocytes at clinically attainable concentrations, is well tolerated, and functions rapidly in vivo.

### Ruxolitinib Inhibits Arrhythmias in Mouse and Patient-Derived Models

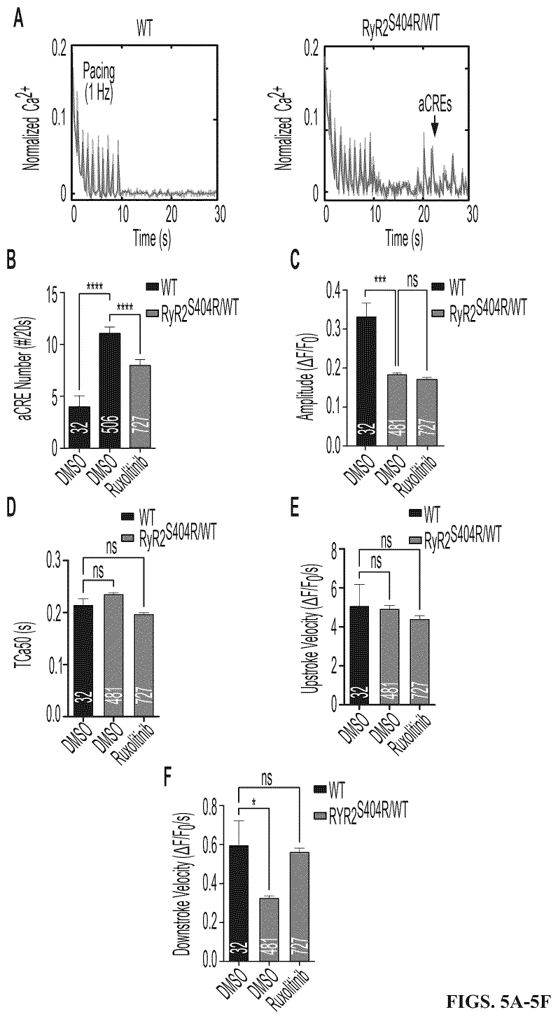

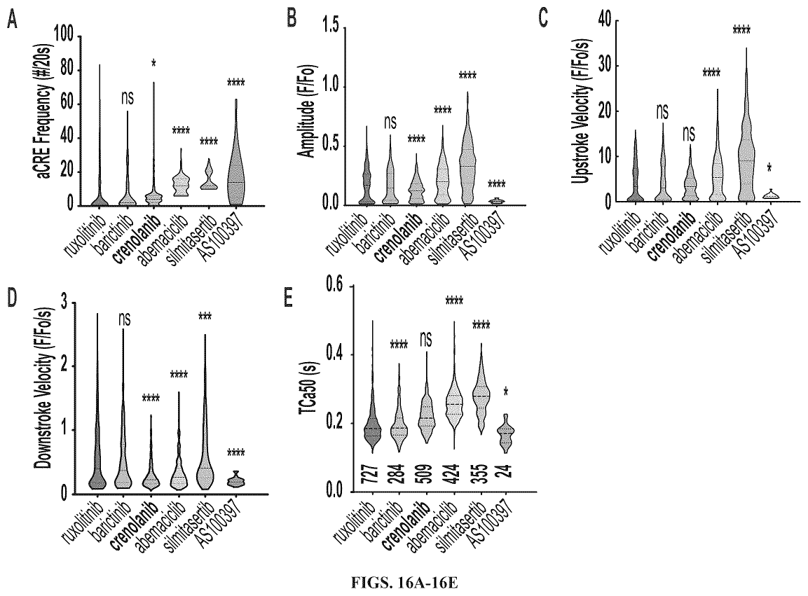

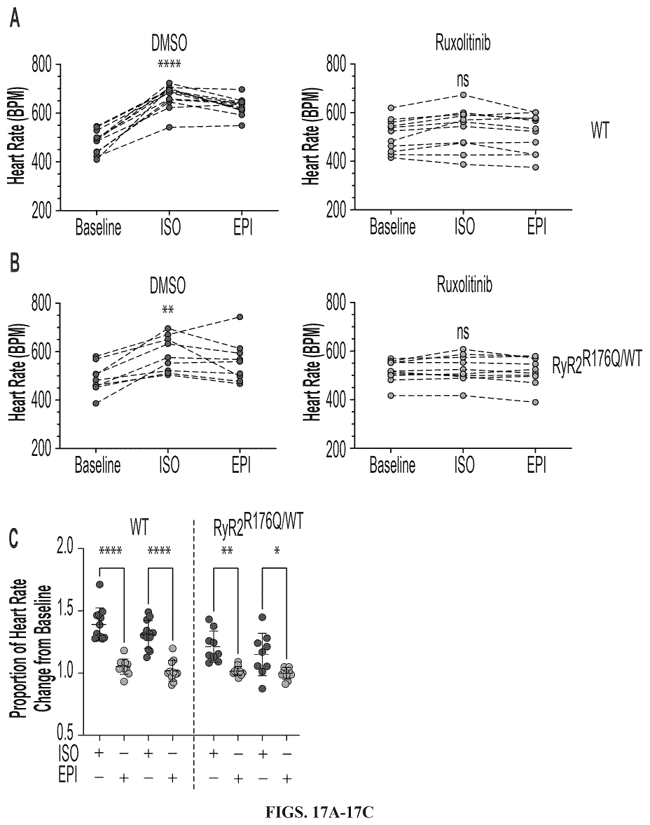

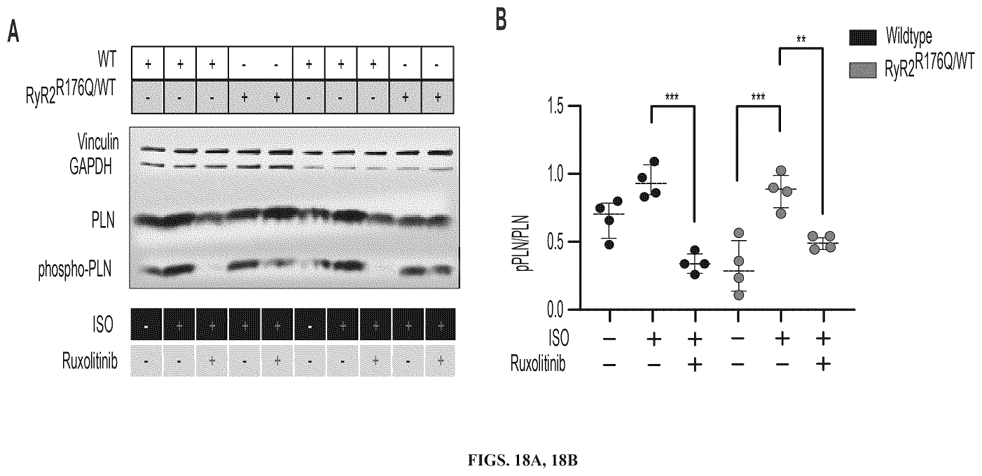

Given its robust inhibition of CaMKII in cellulo and in vivo, it was tested whether ruxolitinib can ameliorate CaMKII-associated cardiac pathology. CPVT was first examined since CaMKII hyperactivity plays an essential role in this arrhythmia (10, 11, 13, 53). A patient with recurrent exercise-induced arrhythmia and a dominant mutation in the ryanodine receptor type 2 (RyR2^{S40R/WT}) was identified for reprogramming into induced pluripotent stem cells (iPSC) (15). After differentiation into functional cardiomyocytes (iPSC-CMs) steady-rate electrical pacing was performed at 1 Hz for 10 seconds followed by cessation of pacing during continuous Ca²⁺ imaging. The frequency of spontaneous abnormal Ca²⁺ release events (aCREs) after the cessation of pacing reflects the cellular mechanism for CPVT and is dramatically increased in iPSC-CMs with pathogenic CPVT mutations (FIG. 5A). Pre-incubation with ruxolitinib effectively suppressed aCREs in RyR2^{S404R/WT} iPSC-CMs compared to vehicle only (FIG. 5B). Automated analysis of Ca²⁺ transients during steady-rate pacing did not reveal any significant effect on peak Ca²⁺ amplitude (FIG. 5C), transient duration (FIG. 5D) or upstroke velocity (FIG. 5E). Additionally, ruxolitinib treatment normalized the downstroke velocity of paced Ca²⁻ transients RyR2^{S404R/RT} iPSC-CMs to wild-type (WT) values (FIG. 5F). The effects of the other drugs identified by the screen were also investigated to test if any were superior to ruxolitinib in reversing this disease phenotype. Among these, ruxolitinib best suppressed aCREs in RyR2^{S404R/WT} iPSC-CMs (FIG. 16A) and least perturbed Ca²⁺ transient parameters (FIGS. 16B-16E).

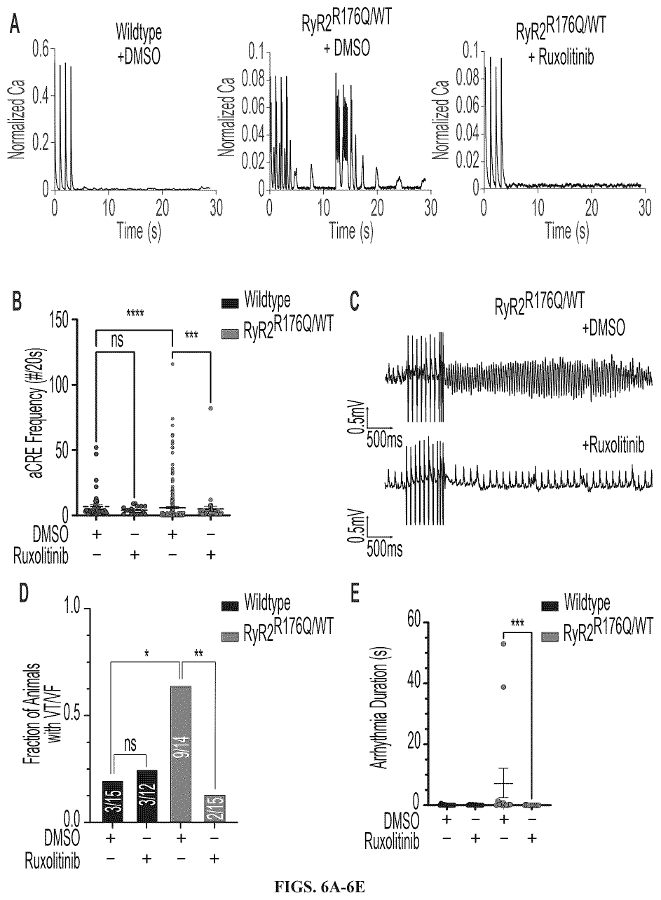

Arrhythmias are emergent derangements of tissues and not just single cells. Thus, a validated mouse model of CPVT was used. Mice with the knock-in mutation Ryr2^{R176Q} have spontaneous and inducible ventricular arrhythmias in response to adrenergic stimulation and ventricular pacing (54). To first test that ruxolitinib could suppress the single cell arrhythmogenic phenotype in the Ryr2^{R176Q/WT} genotype, adult cardiomyocytes were isolated from WT and mutant mice. Like iPSC-CMs derived from a patient suffering from CPVT, Ryr2^{R171Q/WT} adult cardiomyocytes demonstrated abnormal Ca²⁺ release events after the cessation of steady rate pacing (FIG. 6A). Pre-incubation for 10 minutes with 2 μM ruxolitinib significantly reduced the frequency of these events in the Ryr2^{R176Q/WT} adult cardiomyocytes (FIGS. 6A and 6B). To investigate the effects of ruxolitinib on ventricular arrhythmias in CPVT, WT and Ryr2^{R176Q/WT} mice were treated with either ruxolitinib at a dose of 75 mg/kg or vehicle alone (DMSO) by peritoneal injection 10 minutes prior to electrophysiology testing. Programmed electrical stimulation from the right ventricle induced episodes of polymorphic and monomorphic ventricular tachycardia in Ryr2^{R176Q/WT} animals treated with vehicle only (FIG. 6C). However, animals treated with ruxolitinib demonstrated a substantial reduction in the frequency of induced ventricular arrhythmias (FIGS. 6C and 6D). Furthermore, the duration of induced arrhythmias in CPVT mice were also significantly reduced with ruxolitinib treatment (FIG. 6E). Consistent with known effects of CaMKII inhibition (2), ruxolitinib suppressed the adrenergic-induced heart rate increase (FIGS. 17A-17C). No other electrophysiology parameters were negatively affected (Table S3). To confirm that ruxolitinib inhibited CaMKII in our CPVT model whole heart lysates were made from animals immediately after electrophysiology testing. As above, ruxolitinib at a dose of 75 mg/kg significantly inhibited CaMKII-mediated phosphorylation at threonine-17 of phospholamban (FIGS. 18A, 18B).

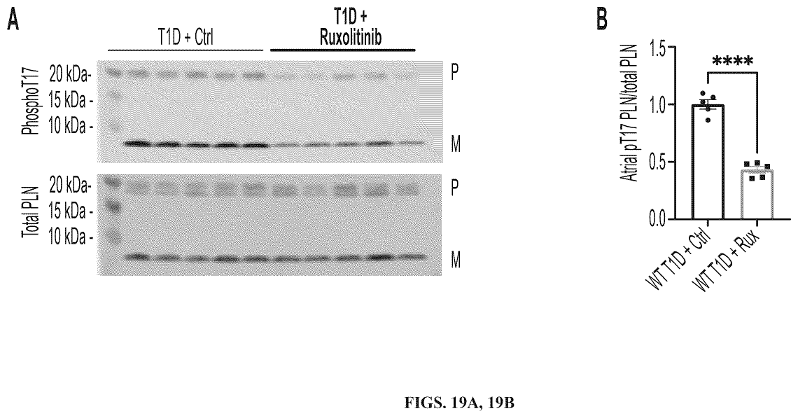

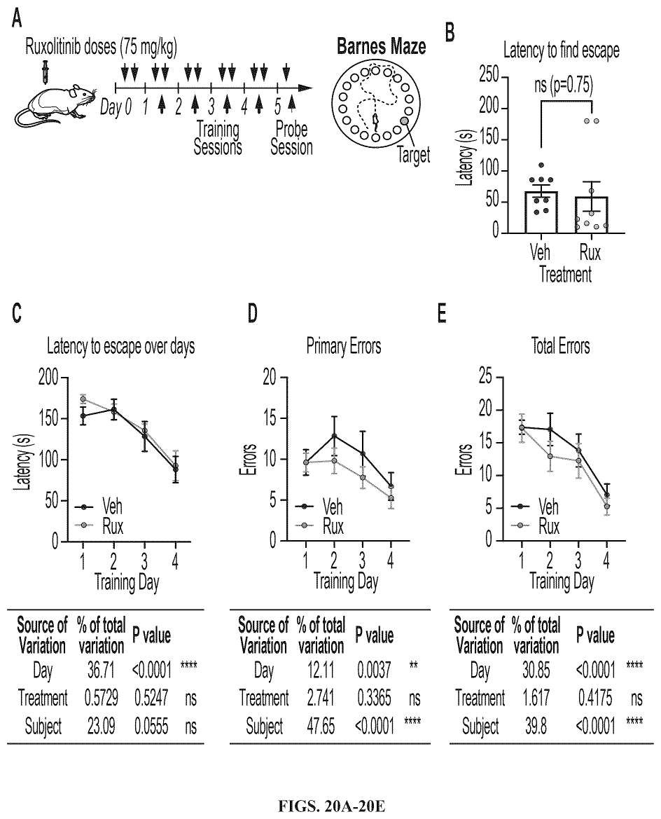

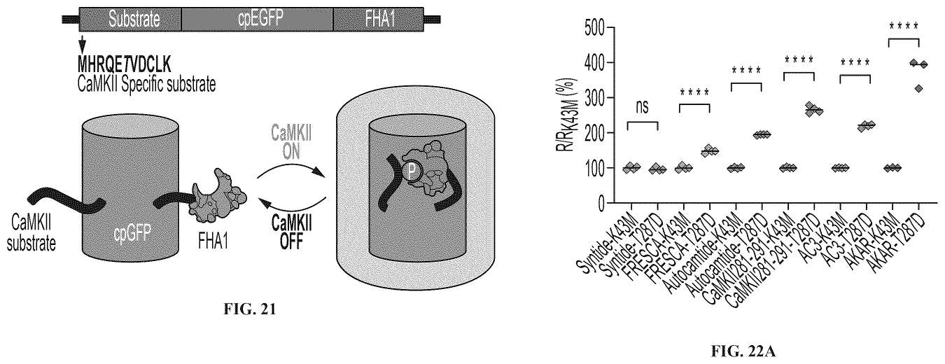

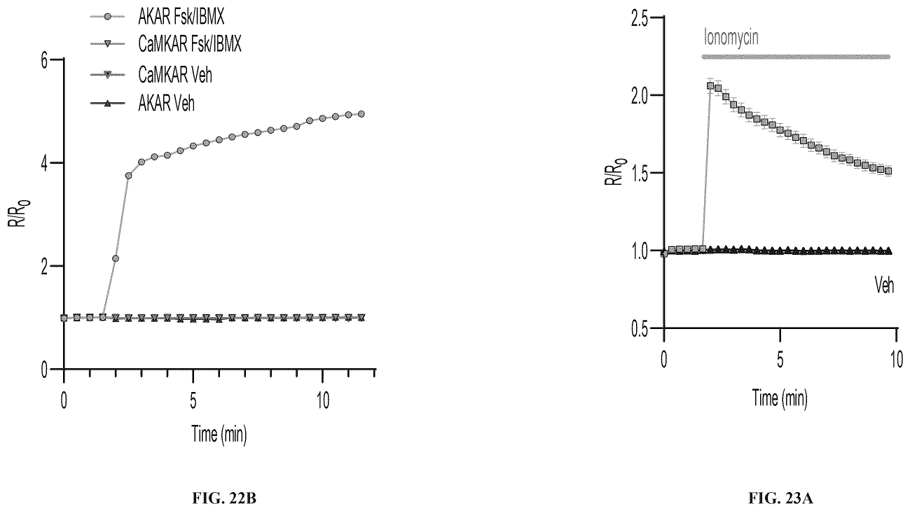

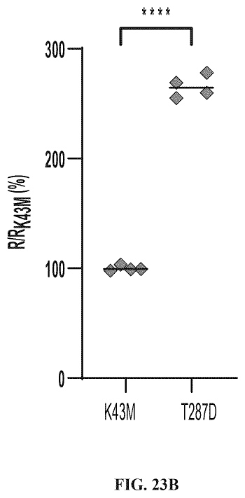

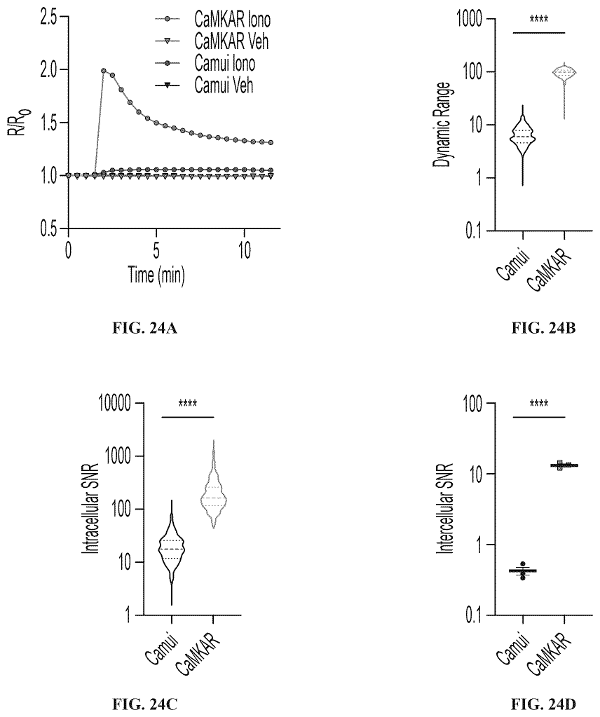

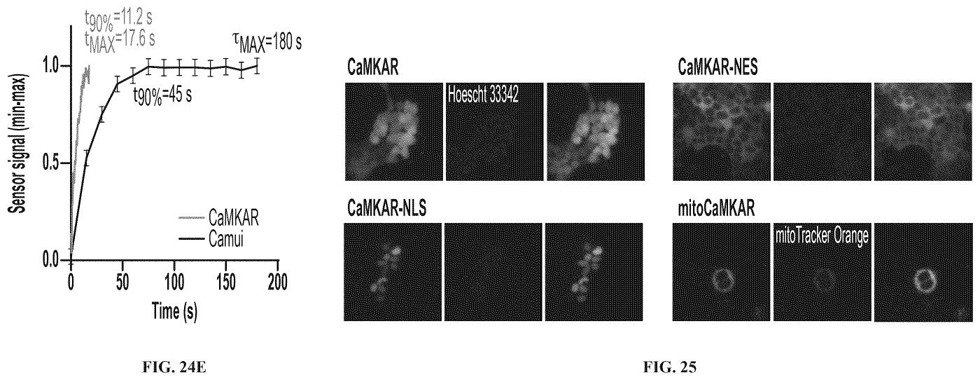

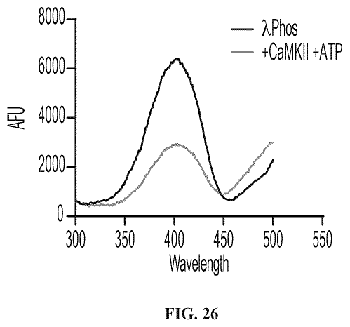

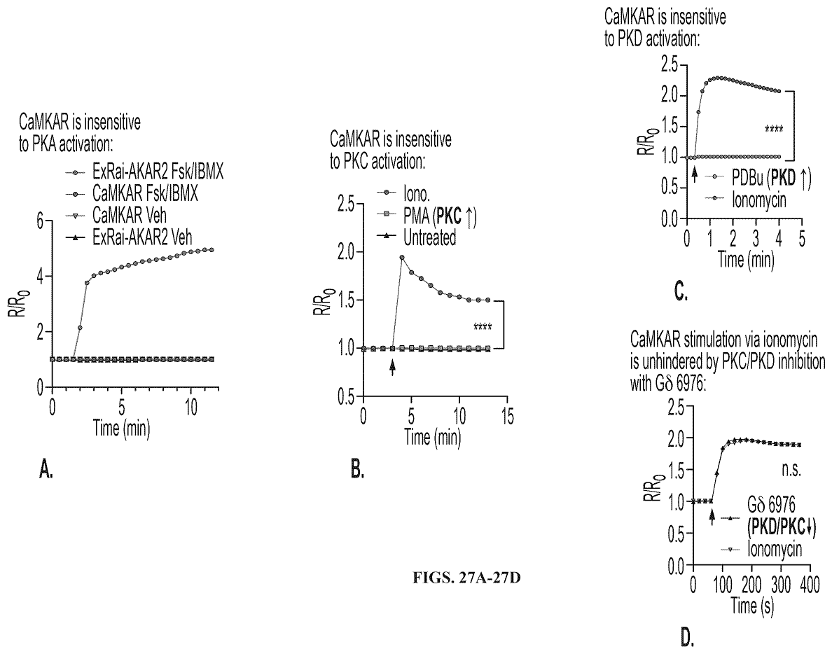

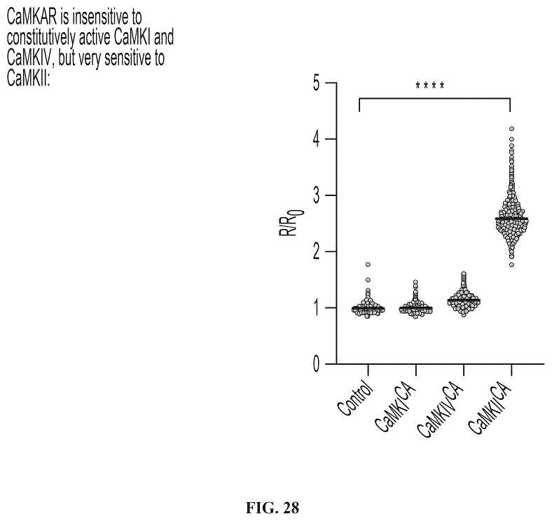

Next, it was tested whether ruxolitinib can prevent and rescue acquired arrhythmia. CaMKII is a pivotal pro-arrhythmic signal in atrial fibrillation (AF) (55-57). A mouse model of AF enhanced by diabetes, a clinical risk factor for AF where hyperglycemia is a known upstream CaMKII activator was previously validated (58, 59). Genetic and chemical interventions that reduced CaMKII activity suppressed AF in diabetic mice (57). 10-minute pretreatment of hyperglycemic mice with 75 mg/kg ruxolitinib abolished pacing-induced AF (FIGS. 7A and B). Examination of threonine-17 phosphorylated phospholamban in atria from these mice confirmed suppression of CaMKII activity in ruxolitinib treated mice (FIGS. 19A, 19B). In O-GlcNAc Transferase (OGT)-overexpressing mice, which develop dilated cardiomyopathy and have increased arrhythmic burden (60), ruxolitinib similarly prevented pacing-induced AF (FIG. 7C). Lastly, rescue of ongoing arrhythmia was tested using CREM-IbAC-X transgenic mice, a validated model of spontaneous AF; by 7 months of age, >70% of CREM-IbAC-X mice develop persistent AF (61, 62). 10 minutes after ruxolitinib treatment, mice showed reduced percent time in AF to 33.63% f 6.5% compared to vehicle which remained at 95.24% f 3.65% (FIGS. 7D and 7E). Collectively, these data support that ruxolitinib can effectively inhibit CaMKII in both atrial and ventricular myocardium and suppress arrhythmogenesis in experimental models of inherited and acquired arrhythmia.

Ruxolitinib does not Lead to Short Term or Spatial Memory Deficits

CaMKII is well known for its role in learning and memory (63). Hence, cognitive off-target effects have been a major criticism against developing CaMKII inhibitors for human use (24). Ruxolitinib is inefficient at crossing the blood-brain barrier, with brain concentration being 29-fold lower than plasma concentration in rats (64). Furthermore, ruxolitinib has been prescribed to patients for over a decade without reported overt cognitive deficits (65). Thus, it was hypothesized that there is a therapeutic window where efficient cardiac inhibition can be achieved without impairing cognition. To test if this was the case in the models, mice were treated with ruxolitinib and subjected them to the novel object recognition test (NORT) and the Y maze spatial memory test (FIG. 8A)—two established behavioral paradigms that test spatial short term memory. These were chosen because CaMKII inhibition is known to affect both short term and spatial memory (66-68), and importantly, numerous studies observe robust deficits in novel object recognition upon CaMKII inhibition (66, 69-72). Neither single dose (1 hour prior) nor multiple doses (twice daily for 7 days) of ruxolitinib (75 or 41 mg/kg) caused significant differences in novel object recognition, as measured by percent time spent with the novel object (FIGS. 8B and 8C). Total distance traveled during testing was similar for both groups, confirming equivalent locomotion (FIGS. 8D and 8E). In the Y maze test, single dosing using the lower dose showed decreased preference for the novel arm (FIG. 8F), but this effect was not seen in the higher dose or in either of the multiple dosing cohorts (FIGS. 8F and 8G).

To assess longer-term spatial memory formation, the effect of ruxolitinib treatment on Barnes maze performance was tested, a test that is also validated to detect CaMKII disruption (67). Mice were initiated on ruxolitinib (150 mg/kg/day) one day prior to training and were maintained on treatment throughout the entire study to ensure continuous exposure to drug (FIG. 20A). At the study endpoint, there was no difference in maze solving latency between the treatment or vehicle groups (FIG. 20B). During the training trials, there was no significant effect in latency, primary errors, and total errors due to ruxolitinib treatment; reassuringly, all three parameters were significantly decreased across days, demonstrating adequate learning (FIGS. 20C to 20D).

These results provide evidence that, at tested doses, ruxolitinib does not lead to detectable impairment of short term or spatial memory, demonstrating feasibility of cardiac CaMKII blockade without memory impairment in mice.

### DISCUSSION

In this work, CaMKAR—a fluorescent CaMKII activity biosensor uniquely suited for high-throughput screening was developed and validated. CaMKAR displays the highest dynamic range of all CaMKII sensors to date, a high degree of specificity, and versatility afforded by ratiometric, intensiometric, and in vitro functionality. These properties were demonstrated by screening a safe-in-human drug collection for CaMKII inhibitors. This revealed that long sought CaMKII inhibitors already exist in the human pharmacopeia and that FDA-approved ruxolitinib is a prime candidate for cardiovascular repurposing.

Ruxolitinib inhibits CaMKII in cardiac cells at clinically achievable concentrations in cells and in vivo. While a 90 mg/kg dose in mice is equivalent to the human maximal prescribed dose (25 mg) (47-49), 41 mg/kg was sufficient to ameliorate CaMKII activity and 75 mg/kg prevented and rescued cardiac arrhythmias. To demonstrate translational potential, CPVT and atrial fibrillation were focused on, two diseases driven by CaMKII hyperactivity (13, 53-57, 73-75). In both patient-derived human cardiomyocytes and in mice, ruxolitinib displayed near complete prevention of arrhythmic phenotypes and a normalization of Ca²⁺ handling. The therapeutic potential shown here can rapidly translate into patients for two main reasons: firstly, CPVT murine knock-in models harboring human disease mutations in RyR2 closely recapitulate human responses to stress (54); secondly, the CaMKII-RyR2-dependent mechanism in CPVT and atrial fibrillation is shared by numerous arrhythmogenic conditions, including inherited (Timothy Syndrome, Duchenne's muscular dystrophy, and Ankyrin B mutations) and acquired (such as glycoside toxicity, heart failure, and alcoholic cardiomyopathy) diseases (5).

Beyond therapeutic repurposing, the results herein add nuance as to whether CaMKII inhibition is safe. While animal studies have repeatedly demonstrated that CaMKII blockade is cardioprotective, pharmaceutical companies have remained cautious about developing CaMKII inhibitors due to its pivotal role in memory (63). The results herein, provide that several drugs already in circulation, taken by thousands-to-millions of patients, inhibit CaMKII. Cognitive testing in mice treated with ruxolitinib showed no overt memory deficit at doses that accomplished robust cardiac CaMKII blockade. This is likely aided by the fact that ruxolitinib displays poor blood-brain barrier penetrance with ˜29-fold higher concentration in plasma than brain (64). This is consistent with the lack of known cognitive side effects in humans. Taken together, the results herein support a reinvigoration of CaMKII inhibitor development and demonstrate that cardiac CaMKII blockade without impairing cognition is achievable with small molecules.

While the data herein provide evidence that ruxolitinib is a prime candidate for cardiac repurposing, excitement for such therapies must be tempered with careful evaluation of ruxolitinib's on-target effects, since our biochemical data suggest that CaMKII and JAK1/2 inhibition will be concomitant. Systemic ruxolitinib is used to treat polycythemia vera, myelofibrosis, and splenomegaly. Due to JAK1/2 inhibition, prolonged treatment can result in herpes zoster infection and reversible anemia and thrombocytopenia (76). Encouragingly, two large scale cohorts of patients taking long-term ruxolitinib (median exposures of 11.1 and of 34.4 months) determined that few patients discontinue treatment due to anemia (<2.6%) or thrombocytopenia (<3.6%)(76). Concern for adverse effects can be abrogated by limiting exposure length, and in agreement with this, several cohorts show that ruxolitinib is well-tolerated at up to 200 mg in healthy volunteers with short-term dosing (42, 77-79). Thus, it is predicted herein that repurposing will be most readily applicable for indications that can both benefit from ruxolitinib's fast onset and require short treatment courses, since these likely offer the optimal tradeoff between CaMKII inhibition and mitigation of undesirable JAK1/2 effects. Three unmet clinical scenarios that match these criteria were identified: 1) post-operative atrial fibrillation, which affects 20-40% of cardiac surgery patients and is independently associated with increased mortality, length of stay, and healthcare cost (80). A majority of cases happen within 3 days and >90% happen by day 6 post-surgery (80, 81). 2) post-myocardial infarction ventricular tachycardia, a significant source of peri-infarct mortality, also occurs predominantly (90%) within the first 48 hours (82). 3) Electrical storm, whereby patients experience multiple episodes of ventricular tachycardia and fibrillation within 24-48 hours. All three of these conditions are exacerbated by CaMKII (83-85), feature β-adrenergic blockade as a mainstay therapy—which has been shown to be insufficient to block CaMKII in human biopsies (14)—and all occur within short and predictable timeframes where a short course of ruxolitinib would be feasible. Other experimental CaMKI-modifying modalities, such as anti-sense oligonucleotides and inhibitor-encoding gene therapy take effect in the order of days-to-weeks, which lessens their utility in these acute indications. Importantly, we demonstrated that ruxolitinib is able to inhibit pre-activated CaMKII and rescue ongoing arrhythmia; this supports a therapeutic role for ruxolitinib as a ‘pill-in-the-pocket’ approach for AF.

Despite its impressive mechanistic response to CaMKII inhibition in animal models, there are no current CaMKII-inhibiting or disease-specific therapies available for CPVT. While the recent addition of flecainide therapy has ameliorated arrhythmic events in many patients, a substantial fraction of CPVT patients is refractory to flecainide therapy and patients still experience breakthrough events and sudden death despite maximal medical therapy and use of implantable cardiac defibrillators (ICDs). In fact, the use of ICDs is associated with potentially lethal ICD storms and death in some patients with exhausted therapies (86). Therefore, although CPVT would require chronic administration, patients resistant to all other therapies would likely find benefit that outweighs ruxolitinib's on-target effects.

CaMKAR's unique properties will enable it to address previously intractable questions. High sensitivity and fast kinetics will allow us to determine the dynamics of pathologic CaMKII activity with high spatiotemporal resolution. Furthermore, CaMKAR can be extensively scaled in cell culture, which will permit more ambitious chemical screens. The molecules identified here can also serve as scaffolds for medicinal chemistry to amplify their CaMKII potency while minimizing their originally intended effects. However, regulatory approval of these derived compounds will likely take decades, which underscores the advantage of exploring ruxolitinib as is. Lastly, while CaMKII is known to drive an extensive number of cardiac pathologies, it has been shown to underpin other illnesses including asthma and cancer (5).

TABLE S1
Comparison of 3 major CaMKII biosensors.

| | Camui | FRESCA | CaMKAR |
|---|---|---|---|
| Maximal amplitude | ~50-80% (34, 41) | 2.37 ± 0.06% (35) | 227 ± 11.1% |
| change | | | |
| Kinetics | ~1-3 min (34, 41) | 1.125 min (35) | 17.6 s |
| (Time-to-peak) | | | |
| Fluorophores | CFP/YFP, | CFP/YFP (35) | Circularly |
| | mNeonGreen/mRuby3 | | permuted GFP |
| | (34, 41) | | |
| Sensing mechanism | Förster resonance | Förster resonance | Fluorescence |
| | energy transfer | energy transfer | intensity increase |
| | (FRET) decrease | (FRET) decrease | secondary to |
| | secondary to | secondary to | CaMKII |
| | conformational | CaMKII | phosphorylation of |
| | change of built- | phosphorylation of | substrate peptide |
| | in CaMKIIα | substrate peptide | |
| Known Compatible | CaMKIIα (34, 41) | CaMKIIγ (35) | CaMKIIα/β/δ/γ |
| CaMKII isoforms | | | |

TABLE S2
Comparison of safety profiles of 5 identified CaMKII inhibitors. N/A = not available.

| | Ruxolitinib | Baricitinib | Abemaciclib | Silmitasertib | Crenolanib |
|---|---|---|---|---|---|
| Original | JAK1/2 | JAK1/2 | CDK4/6 | CK2 | FLT3, |
| target(s) | | | | | PDGFRα, |
| | | | | | PDGFRβ, |
| | | | | | C-Kit |
| Indications | Atopic dermatitis, | Rheumatoid | HR-positive, | Cholangiocarcinoma, | Acute |
| | Vitiligo, | Arthritis, | HER2- | Medulloblastoma | myeloid |
| | Polycythemia, | Alopecia | negative | | leukemia, |
| | splenomegaly, | areata, | advanced or | | GIST, |
| | myelofibrosis | COVID-19 | metastatic | | glioma |
| | | | breast cancer | | |
| Notable | Reversible | Opportunistic | Neutropenia, | Anemia, diarrhea, | Vomiting, |
| adverse | thrombocytopenia | respiratory | interstitial | fatigue, | nausea, |
| effects | and anemia, | infections, | lung disease, | thrombocytopenia | diarrhea, |
| | herpes zoster | neutropenia, | hepatoxicity, | (93) | transaminitis |
| | infection (76) | pulmonary | embryo-fetal | | (45, 94, 95) |
| | | embolism, | toxicity, | | |
| | | thrombosis | thrombosis | | |
| | | (90) | (91, 92) | | |
| Maximum | FDA-approved: | FDA-approved: | 200 mg BID | 1000 mg BID | 100 mg TID |
| dose used | 25 mg BID (96) | 4 mg QD (97) | (99) | (93) | (100) |
| in humans | Healthy | Healthy | | | |
| | volunteers: | volunteers: | | | |
| | 200 mg QD (76) | 40 mg (98) | | | |
| Treatment | Median 2.9 | Median 4.6 | Median 14 | 21 days | 9 weeks |
| duration | years, | years (90) | months | (93) | (102) |
| | (COMFORT | | (monarchE | | |
| | trials) (76) | | trial) (101) | | |
| Year of | 2011 | 2018 | 2017 | FDA orphan drug | N/A |
| FDA- | | | | status: 2017 | |
| approval | | | | | |
| Agency | FDA, EMA | FDA, EMA, | FDA, EMA, | FDA orphan drug | None |
| approvals | PMDA | PMDA | PMDA | status (Phase II | (Phase II |
| | | | | trials) | trials) |
| CNS | 3.5% (64) | 20% (103) | 11-16.6% | N/A | N/A |
| penetrance | | | (104) | | |

TABLE S3
Electrophysiologic parameters in mice treated with ruxolitinib and either
isoproterenol (ISO) or epinephrine (EPI). All parameters are in msec.

| | | DMSO | | | Ruxolitinib | | |
|---|---|---|---|---|---|---|---|
| | | Baseline | ISO | EPI | Baseline | ISO | EPI |
| WT | PR | 40.31 ± 2 | 36.73 ± 1.8 | 120.7 ± 84 | 43.4 ± 4 | 43.8 ± 4 | 45.9 ± 2.7 |
| | QRS | 10.8 ± 0.8 | 11.31 ± 1 | 9.9 ± 1 | 13.2 ± 2 | 11.5 ± 1 | 12.5 ± 1 |
| | QTc | 39.74 ± 6 | 41.9 ± 3 | 46.4 ± 6.2 | 48.6 ± 6 | 49.2 ± 4 | 55.7 ± 8 |
| RyR^{R176Q/WT} | PR | 41.1 ± 2 | 36.9 ± 3 | 40.02 ± 3 | 39.5 ± 1 | 43.1 ± 3 | 42.2 ± 4 |
| | QRS | 11.03 ± 1 | 8.8 ± 0.6 | 11.1 ± 1 | 9.3 ± 0.7 | 11.6 ± 1.2 | 11.5 ± 0.8 |
| | QTc | 38.5 ± 4 | 46.1 ± 5 | 50.7 ± 5 | 36.5 ± 5 | 51.6 ± 5 | 49.5 ± 4 |

### REFERENCES

1. G. A. Roth, et al. Global Burden of Cardiovascular Diseases and Risk Factors, 1990-2019. J. Am. Coll. Cardiol. 76, 2982-3021 (2020).
2. Y. Wu, Z. Gao, B. Chen, O. M. Koval, M. V. Singh, X. Guan, T. J. Hund, W. Kutschke, S. Sarma, I. M. Grumbach, X. H. T. Wehrens, P. J. Mohler, L.-S. Song, M. E. Anderson, Calmodulin kinase II is required for fight or flight sinoatrial node physiology. Proc. Natl. Acad Sci. (2009), doi:10.1073/pnas.0806422106.
3. M. M. Kreusser, L. et al. Cardiac CaM Kinase II Genes 6 and 7 Contribute to Adverse Remodeling but Redundantly Inhibit Calcineurin-Induced Myocardial Hypertrophy. Circulation 130, 1262-1273 (2014).
4. J. Beckendorf, M. M. G. van den Hoogenhof, J. Backs, Physiological and unappreciated roles of CaMKII in the heart. Basic Res. Cardiol. 113, 29 (2018).
5. O. E. Reyes Gaido, L. J. Nkashama, K. L. Schole, Q. Wang, P. Umapathi, O. O. Mesubi, K. Konstantinidis, E. D. Luczak, M. E. Anderson, CaMKII as a Therapeutic Target in Cardiovascular Disease. Annu. Rev. Pharmacol. Toxicol. 63, 249-272 (2023).
6. M. L. A. Joiner, et al. CaMKII determines mitochondrial stress responses in heart. Nature (2012), doi:10.1038/nature11444.
7. R. Zhang, M. S. C. Khoo, Y. Wu, Y. Yang, C. E. Grueter, G. Ni, E. E. Price, W. Thiel, S. Guatimosim, L. S. Song, E. C. Madu, A. N. Shah, T. A. Vishnivetskaya, J. B. Atkinson, V. V. Gurevich, G. Salama, W. J. Lederer, R. J. Colbran, M. E. Anderson, Calmodulin kinase II inhibition protects against structural heart disease. Nat. Med. (2005), doi:10.1038/nm1215.
8. C. E. Grueter, R. J. Colbran, M. E. Anderson, CaMKII, an emerging molecular driver for calcium homeostasis, arrhythmias, and cardiac dysfunction/. Mol. Med. (2007), doi:10.1007/s00109-006-0125-6.
9. B. J. He, M. L. A. Joiner, M. V. Singh, E. D. Luczak, P. D. Swaminathan, O. M. Koval, W. Kutschke, C. Allamargot, J. Yang, X. Guan, K. Zimmerman, I. M. Grumbach, R. M. Weiss, D. R. Spitz, C. D. Sigmund, W. M. Blankesteijn, S. Heymans, P. J. Mohler, M. E. Anderson, Oxidation of CaMKII determines the cardiotoxic effects of aldosterone. Nat. Med. (2011), doi:10.1038/nm.2506.
10. E. Di Pasquale, F. Lodola, M. Miragoli, M. Denegri, J. E. Avelino-Cruz, M. Buonocore, H. Nakahama, P. Portararo, R. Bloise, C. Napolitano, G. Condorelli, S. G. Priori, CaMKII inhibition rectifies arrhythmic phenotype in a patient-specific model of catecholaminergic polymorphic ventricular tachycardia. Cell Death Dis. (2013), doi:10.1038/cddis.2013.369.
11. K. Konstantinidis, V. J. Bezzerides, L. Lai, H. M. Isbell, A. C. Wei, Y. Wu, M. C. Viswanathan, I. D. Blum, J. M. Granger, D. Heims-Waldron, D. Zhang, E. D. Luczak, K. R. Murphy, F. Lu, D. H. Gratz, B. Manta, Q. Wang, Q. Wang, A. L. Kolodkin, V. N. Gladyshev, T. J. Hund, W. T. Pu, M. N. Wu, A. Cammarato, M. A. Bianchet, M. A. Shea, R. L. Levine, M. E. Anderson, MICAL1 constrains cardiac stress responses and protects against disease by oxidizing CaMKII. J. Clin. Invest. (2020), doi:10.1172/JCI133181.
12. Y. Wu, Q. Wang, N. Feng, J. M. Granger, M. E. Anderson, Myocardial death and dysfunction after ischemia-reperfusion injury require CaMKIIδ oxidation. Sci. Rep. (2019), doi:10.1038/s41598-019-45743-6.
13. V. J. Bezzerides, A. Caballero, S. Wang, Y. Ai, R. J. Hylind, F. Lu, D. A. Heims-Waldron, K. D. Chambers, D. Zhang, D. J. Abrams, W. T. Pu, Gene Therapy for Catecholaminergic Polymorphic Ventricular Tachycardia by Inhibition of Ca²⁺/Calmodulin-Dependent Kinase II. Circulation (2019), doi:10.1161/CIRCULATIONAHA.118.038514.
14. M. Dewenter, S. Neef, C. Vettel, S. Limmle, C. Beushausen, L. C. Zelarayan, S. Katz, A. von der Lieth, S. Meyer-Roxlau, S. Weber, T. Wieland, S. Sossalla, J. Backs, J. H. Brown, L. S. Maier, A. El-Armouche, Calcium/Calmodulin-Dependent Protein Kinase II Activity Persists During Chronic β-Adrenoceptor Blockade in Experimental and Human Heart Failure. Circ. Heart Fail. 10, e003840 (2017).
15. H. Tokumitsu, T. Chijiwa, M. Hagiwara, A. Mizutani, M. Terasawa, H. Hidaka, KN-62, 1-[N,O-bis(5-isoquinolinesulfonyl)-N-methyl-L-tyrosyl]-4-phenylpiperazi ne, a specific inhibitor of Ca2+/calmodulin-dependent protein kinase I. J. Biol. Chem. 265, 4315-4320 (1990).
16. M. Sumi, K. Kiuchi, T. Ishikawa, A. Ishii, M. Hagiwara, T. Nagatsu, H. Hidaka, The newly synthesized selective Ca2+/calmodulin dependent protein kinase H inhibitor KN-93 reduces dopamine contents in PC12h cells. Biochem. Biophys. Res. Commun. 181, 968-975 (1991).
17. M. Vila-Petroff, M. A. Salas, M. Said, C. A. Valverde, L. Sapia, E. Portiansky, R. J. Hajjar, E. G. Kranias, C. Mundifia-Weilenmann, A. Mattiazzi, CaMKII inhibition protects against necrosis and apoptosis in irreversible ischemia-reperfusion injury. Cardiovasc. Res. 73, 689-698 (2007).
18. J. Ledoux, D. Chartier, N. Leblanc, Inhibitors of calmodulin-dependent protein kinase are nonspecific blockers of voltage-dependent K+ channels in vascular myocytes. J. Pharmacol. Exp. Ther. 290, 1165-1174 (1999).
19. S. Rezazadeh, T. W. Claydon, D. Fedida, KN-93 (2-[N-(2-hydroxyethyl)]-N-(4-methoxybenzenesulfonyl)]amino-N-(4-chlorocinnamyl)-N-methylbenzylamine), a calcium/calmodulin-dependent protein kinase II inhibitor, is a direct extracellular blocker of voltage-gated potassium channels. J. Pharmacol. Exp. Ther. 317, 292-299 (2006).
20. B. Hegyi, Y. Chen-Izu, Z. Jian, R. Shimkunas, L. T. Izu, T. Banyasz, KN-93 inhibits IKr in mammalian cardiomyocytes. J. Mol. Cell. Cardiol. 89, 173-176 (2015).
21. M. H. Wong, A. B. Samal, M. Lee, J. Vlach, N. Novikov, A. Niedziela-Majka, J. Y. Feng, D. O. Koltun, K. M. Brendza, H. J. Kwon, B. E. Schultz, R. Sakowicz, J. S. Saad, G. A. Papalia, The KN-93 Molecule Inhibits Calcium/Calmodulin-Dependent Protein Kinase II (CaMKII) Activity by Binding to Ca2+/CaM. J. Mol. Biol. 431, 1440-1459 (2019).
22. B. H. Chang, S. Mukherji, T. R. Soderling, Characterization of a calmodulin kinase 1 inhibitor protein in brain. Proc. Natl. Acad. Sci. 95, 10890-5 (1998).
23. R. S. Vest, K. D. Davies, H. O'Leary, J. D. Port, K. U. Bayer, Dual Mechanism of a Natural CaMKII Inhibitor. Mol. Biol. Cell (2007), doi:10.1091/mbc.e07-02-0185.
24. P. Pellicena, H. Schulman, CaMKII inhibitors: From research tools to therapeutic agents Front. Pharmacol. (2014), doi:10.3389/fphar.2014.00021.
25. S. Neef, A. Steffens, P. Pellicena, J. Mustroph, S. Lebek, K. R. Ort, H. Schulman, L. S. Maier, Improvement of cardiomyocyte function by a novel pyrimidine-based CaMKII-inhibitor. J. Mol. Cell. Cardiol. 115, 73-81 (2018).
26. S. Lebek, A. Plöβl, M. Baier, J. Mustroph, D. Tarnowski, C. M. Licht, S. Schopka, B. Florchinger, C. Schmid, Y. Zausig, N. Pagratis, B. Marchand, D. O. Koltun, W. K. Hung, S. Ahmadyar, L. Belardinelli, L. S. Maier, S. Wagner, The novel CaMKII inhibitor GS-680 reduces diastolic SR Ca leak and prevents CaMKII-dependent pro-arrhythmic activity. J. Mol. Cell. Cardiol. 118, 159-168 (2018).
27. P. Beauverger, M.-L. Ozoux, G. Bégis, V. Glénat, V. Briand, M.-C. Philippo, C. Daveu, G. Tavares, S. Roy, A. Corbier, P. Briand, O. Dorchies, A.-L. Bauchet, E. Nicolai, O. Duclos, D. Tamarelle, M.-P. Pruniaux, A. J. Muslin, P. Janiak, Reversion of cardiac dysfunction by a novel orally available calcium/calmodulin-dependent protein kinase II inhibitor, RA306, in a genetic model of dilated cardiomyopathy. Cardiovasc. Res. 116, 329-338 (2020).
28. J. Zhang, R. Liang, K. Wang, W. Zhang, M. Zhang, L. Jin, P. Xie, W. Zheng, H. Shang, Q. Hu, J. Li, G. Chen, F. Wu, F. Lan, L. Wang, S.-Q. Wang, Y. Li, Y. Zhang, J. Liu, F. Lv, X. Hu, R.-P. Xiao, X. Lei, Y. Zhang, Novel CaMKII-S Inhibitor Hesperadin Exerts Dual Functions to Ameliorate Cardiac Ischemia/Reperfusion Injury and Inhibit Tumor Growth. Circulation 145, 1154-1168 (2022).
29. N. R. Lim, C. J. Thomas, L. S. Silva, Y. Y. Yeap, S. Yap, J. R. Bell, L. M. D. Delbridge, M. A. Bogoyevitch, O. L. Woodman, S. J. Williams, C. N. May, D. C. H. Ng, Cardioprotective 3′,4′-dihydroxyflavonol attenuation of JNK and p38MAPK signalling involves CaMKII inhibition. Biochem. J. 456, 149-161 (2013).
30. A. J. Boyle, C. Schultz, J. B. Selvanayagam, S. Moir, R. Kovacs, N. Dib, D. Zlotnick, M. Al-Omary, S. Sugito, A. Selvarajah, N. Collins, G. McLachlan, Calcium/Calmodulin-Dependent Protein Kinase II Delta Inhibition and Ventricular Remodeling After Myocardial Infarction: A Randomized Clinical Trial. JAMA Cardiol. 6, 762-768 (2021).
31. M. E. Anderson, To Be or Not to Be a CaMKII Inhibitor?JAMA Cardiol. 6, 769-770 (2021).
32. J. Lisman, R. Yasuda, S. Raghavachari, Mechanisms of CaMKII action in long-term potentiation. Nat. Rev. Neurosci. 13, 169-182 (2012).
33. K. Takao, K.-I. Okamoto, T. Nakagawa, R. L. Neve, T. Nagai, A. Miyawaki, T. Hashikawa, S. Kobayashi, Y. Hayashi, Visualization of synaptic Ca2+/calmodulin-dependent protein kinase II activity in living neurons. J. Neurosci. Off J. Soc. Neurosci. 25, 3107-3112 (2005).
34. J. R. Erickson, R. Patel, A. Ferguson, J. Bossuyt, D. M. Bers, FRET-based sensor Camui provides new insight into mechanisms of CaMKII activation in intact cardiomyocytes. Circ. Res. 109, 729-738 (2011).
35. G. Ardestani, M. C. West, T. J. Maresca, R. A. Fissore, M. M. Stratton, FRET-based sensor for CaMKII activity (FRESCA): A useful tool for assessing CaMKII activity in response to Ca(2+) oscillations in live cells. J Biol Chem 294, 11876-11891 (2019).
36. A. I. Kostyuk, A. D. Demidovich, D. A. Kotova, V. V. Belousov, D. S. Bilan, Circularly Permuted Fluorescent Protein-Based Indicators: History, Principles, and Classification. Int. J. Mol. Sci. 20, 4200 (2019).
37. S. Mehta, Y. Zhang, R. H. Roth, J. Zhang, A. Mo, B. Tenner, R. L. Huganir, J. Zhang, Single-fluorophore Biosensors for Sensitive and Multiplexed Detection of Signaling Activities. Nat. Cell Biol. 20, 1215-1225 (2018).
38. J.-F. Zhang, B. Liu, I. Hong, A. Mo, R. H. Roth, B. Tenner, W. Lin, J. Z. Zhang, R. S. Molina, M. Drobizhev, T. E. Hughes, L. Tian, R. L. Huganir, S. Mehta, J. Zhang, An ultrasensitive biosensor for high-resolution kinase activity imaging in awake mice. Nat. Chem. Biol. 17, 39-46 (2021).
39. D. E. Levy, H. Schulman, B. R. Paraselli, N. S. Kumar, B. Dabbugoddu, C. Balasubramanyam, CaMKII inhibitors and uses thereof (2017) (available at https://patents.google.com/patent/US20170247374A1/en).
40. D. E. Levy, H. Schulman, B. R. Paraselli, N. S. Kumar, B. Dabbugoddu, C. Balasubramanyam, CaMKII inhibitors and uses thereof (2020) (available at https://patents.google.com/patent/US20200385383A1/en?inventor=Howard+Schulman).
41. B. T. Bajar, E. S. Wang, A. J. Lam, B. B. Kim, C. L. Jacobs, E. S. Howe, M. W. Davidson, M. Z. Lin, J. Chu, Improving brightness and photostability of green and red fluorescent proteins for live cell imaging and FRET reporting. Sci. Rep. 6, 20889 (2016).
42. N. Punwani, S. Yeleswaram, X. Chen, J. Bowman, M. Soloviev, W. Williams, Evaluation of the effect of ruxolitinib on cardiac repolarization: A thorough QT study. Clin. Pharmacol. Drug Dev. 3, 207-214 (2014).
43. X. Zhao, X. Y. Sheng, C. D. Payne, X. Zhang, F. Wang, Y. M. Cui, Pharmacokinetics, Safety, and Tolerability of Single- and Multiple-Dose Once-Daily Baricitinib in Healthy Chinese Subjects: A Randomized Placebo-Controlled Study. Clin. Pharmacol. Drug Dev. 9, 952-960 (2020).
44. B. Zhong, O. Campagne, R. Salloum, T. Purzner, C. F. Stewart, LC-MS/MS method for quantitation of the CK2 inhibitor silmitasertib (CX-4945) in human plasma, CSF, and brain tissue, and application to a clinical pharmacokinetic study in children with brain tumors. J. Chromatogr. B Analyt. Technol. Biomed. Life. Sci. 1152, 122254 (2020).
45. J. C. Panetta, S. D. Baker, H. M. Kantadjian, C. Stewart, B. Pond, M. Macaraeg, T. Makinde, S. Vali, N. Daver, A. Ramachandran, R. Collins, J. E. Cortes, Population Pharmacokinetics of Crenolanib, a Type I FLT3 Inhibitor, in Patients with Relapsed/Refractory AML. Blood 126, 3695 (2015).
46. A. Patnaik, et al., Efficacy and Safety of Abemaciclib, an Inhibitor of CDK4 and CDK6, for Patients with Breast Cancer, Non-Small Cell Lung Cancer, and Other Solid Tumors. Cancer Discov. 6, 740-753 (2016).
47. N. Mollé, S. Krichevsky, P. Kermani, R. T. Silver, E. Ritchie, J. M. Scandura, Ruxolitinib can cause weight gain by blocking leptin signaling in the brain via JAK2/STAT3. Blood 135, 1062-1066 (2020).
48. P. M. Reeves, M. A. Abbaslou, F. R. W. Kools, K. Vutipongsatorn, X. Tong, C. Gavegano, R. F. Schinazi, M. C. Poznansky, Ruxolitinib sensitizes ovarian cancer to reduced dose Taxol, limits tumor growth and improves survival in immune competent mice. Oncotarget 8, 94040-94053 (2017).
49. R. Das, P. Guan, L. Sprague, K. Verbist, P. Tedrick, Q. A. An, C. Cheng, M. Kurachi, R. Levine, E. J. Wherry, S. W. Canna, E. M. Behrens, K. E. Nichols, Janus kinase inhibition lessens inflammation and ameliorates disease in murine models of hemophagocytic lymphohistiocytosis. Blood 127, 1666-1675 (2016).
50. M. A. Movsesian, M. Nishikawa, R. S. Adelstein, Phosphorylation of phospholamban by calcium-activated, phospholipid-dependent protein kinase. Stimulation of cardiac sarcoplasmic reticulum calcium uptake. J. Biol. Chem. 259, 8029-8032 (1984).
51. H. K. Simmerman, J. H. Collins, J. L. Theibert, A. D. Wegener, L. R. Jones, Sequence analysis of phospholamban. Identification of phosphorylation sites and two major structural domains. J. Biol. Chem. 261, 13333-13341 (1986).
52. A. Mattiazzi, E. Kranias, The role of CaMKII regulation of phospholamban activity in heart disease. Front. Pharmacol. 5 (2014) (available at https://www.frontiersin.org/articles/10.3389/fphar.2014.00005).
53. N. Liu, Y. Ruan, M. Denegri, T. Bachetti, Y. Li, B. Colombi, C. Napolitano, W. A. Coetzee, S. G. Priori, Calmodulin kinase II inhibition prevents arrhythmias in RyR2(R4496C+/−) mice with catecholaminergic polymorphic ventricular tachycardia. J. Mol. Cell. Cardiol. 50, 214-222 (2011).
54. P. J. Kannankeril, B. M. Mitchell, S. A. Goonasekera, M. G. Chelu, W. Zhang, S. Sood, D. L. Kearney, C. I. Danila, M. De Biasi, X. H. T. Wehrens, R. G. Pautler, D. M. Roden, G. E. Taffet, R. T. Dirksen, M. E. Anderson, S. L. Hamilton, Mice with the R176Q cardiac ryanodine receptor mutation exhibit catecholamine-induced ventricular tachycardia and cardiomyopathy. Proc. Natl. Acad. Sci. U.S.A 103, 12179-12184 (2006).
55. M. G. Chelu, S. Sarma, S. Sood, S. Wang, R. J. van Oort, D. G. Skapura, N. Li, M. Santonastasi, F. U. Müller, W. Schmitz, U. Schotten, M. E. Anderson, M. Valderribano, D. Dobrev, X. H. T. Wehrens, Calmodulin kinase II-mediated sarcoplasmic reticulum Ca2+ leak promotes atrial fibrillation in mice. J. Clin. Invest. 119, 1940-1951 (2009).
56. A. Purohit, A. G. Rokita, X. Guan, B. Chen, O. M. Koval, N. Voigt, S. Neef, T. Sowa, Z. Gao, E. D. Luczak, H. Stefansdottir, A. C. Behunin, N. Li, R. N. El-Accaoui, B. Yang, P. D. Swaminathan, R. M. Weiss, X. H. T. Wehrens, L.-S. Song, D. Dobrev, L. S. Maier, M. E. Anderson, Oxidized Ca2+/Calmodulin-Dependent Protein Kinase II Triggers Atrial Fibrillation. Circulation 128, 1748-1757 (2013).
57. O. O. Mesubi, A. G. Rokita, N. Abrol, Y. Wu, B. Chen, Q. Wang, J. M. Granger, A. Tucker-Bartley, E. D. Luczak, K. R. Murphy, P. Umapathi, P. S. Banerjee, T. N. Boronina, R. N. Cole, L. S. Maier, X. H. Wehrens, J. L. Pomerantz, L.-S. Song, R. S. Ahima, G. W. Hart, N. E. Zachara, M. E. Anderson, Oxidized CaMKII and O-GlcNAcylation cause increased atrial fibrillation in diabetic mice by distinct mechanisms. J. Clin. Invest. 131 (2021), doi:10.1172/JCI95747.
58. W. B. Kannel, R. D. Abbott, D. D. Savage, P. M. McNamara, Epidemiologic features of chronic atrial fibrillation: the Framingham study. N. Engl. J. Med. 306, 1018-1022 (1982).
59. J. R. Erickson, L. Pereira, L. Wang, G. Han, A. Ferguson, K. Dao, R. J. Copeland, F. Despa, G. W. Hart, C. M. Ripplinger, D. M. Bers, Diabetic hyperglycaemia activates CaMKII and arrhythmias by O-linked glycosylation. Nature 502, 372-6 (2013).
60. P. Umapathi, P. S. Banerjee, N. E. Zachara, N. Abrol, Q. Wang, O. O. Mesubi, E. D. Luczak, Y. Wu, J. M. Granger, A.-C. Wei, O. E. Reyes Gaido, L. Florea, C. C. Talbot Jr., G. W. Hart, M. E. Anderson, Excessive O—GlcNAcylation Causes Heart Failure and Sudden Death. Circulation, CIRCULATIONAHA.120.051911 (2021).
61. F. U. Müller, G. Lewin, H. A. Baba, P. Boknik, L. Fabritz, U. Kirchhefer, P. Kirchhof, K. Loser, M. Matus, J. Neumann, B. Riemann, W. Schmitz, Heart-directed expression of a human cardiac isoform of cAMP-response element modulator in transgenic mice. J. Riol. Chem. 280, 6906-6914 (2005).
62. N. Li, D. Y. Chiang, S. Wang, Q. Wang, L. Sun, N. Voigt, J. L. Respress, S. Ather, D. G. Skapura, V. K. Jordan, F. T. Horrigan, W. Schmitz, F. U. Müller, M. Valderrabano, S. Nattel, D. Dobrev, X. H. T. Wehrens, Ryanodine-Receptor Mediated Calcium Leak Drives Progressive Development of an Atrial Fibrillation Substrate in a Transgenic Mouse Model. Circulation 129, 1276-1285 (2014).
63. K. U. Bayer, H. Schulman, CaM kinase: Still inspiring at 40. Neuron 103, 380-394 (2019).
64. Q. Zhang, Y. Zhang, S. Diamond, J. Boer, J. J. Harris, Y. Li, M. Rupar, E. Behshad, C. Gardiner, P. Collier, P. Liu, T. Burn, R. Wynn, G. Hollis, S. Yeleswaram, The Janus Kinase 2 Inhibitor Fedratinib Inhibits Thiamine Uptake: A Putative Mechanism for the Onset of Wernicke's Encephalopathy. Drug Metab. Dispos. 42, 1656-1662 (2014).
65. G. L. Plosker, Ruxolitinib: A Review of Its Use in Patients with Myelofibrosis. Drugs 75, 297-308 (2015).
66. H. Wang, R. Feng, L. P. Wang, F. Li, X. Cao, J. Z. Tsien, CaMKII activation state underlies synaptic labile phase of LTP and short-term memory formation. Curr. Biol. CB 18, 1546-1554 (2008).
67. M. E. Bach, R. D. Hawkins, M. Osman, E. R. Kandel, M. Mayford, Impairment of spatial but not contextual memory in CaMKII mutant mice with a selective loss of hippocampal LTP in the range of the theta frequency. Cell 81, 905-915 (1995).
68. G. Zalcman, N. Federman, A. Romano, CaMKII Isoforms in Learning and Memory: Localization and Function. Front. Mol. Neurosci. (2018), doi:10.3389/fnmol.2018.00445.
69. H. Wang, E. Shimizu, Y.-P. Tang, M. Cho, M. Kyin, W. Zuo, D. A. Robinson, P. J. Alaimo, C. Zhang, H. Morimoto, M. Zhuo, R. Feng, K. M. Shokat, J. Z. Tsien, Inducible protein knockout reveals temporal requirement of CaMKII reactivation for memory consolidation in the brain. Proc. Natl. Acad. Sci. 100, 4287-4292 (2003).
70. S. Moriguchi, H. Tagashira, Y. Sasaki, J. Z. Yeh, H. Sakagami, T. Narahashi, K. Fukunaga, CaMKII activity is essential for improvement of memory-related behaviors by chronic rivastigmine treatment. J. Neurochem. 128, 927-937 (2014).
71. A. D. Bachstetter, S. J. Webster, T. Tu, D. S. Goulding, J. Haiech, D. M. Watterson, L. J. Van Eldik, Generation and Behavior Characterization of CaMKII$ Knockout Mice. PLoS ONE 9, e105191 (2014).
72. N. Federman, V. de la Fuente, G. Zalcman, N. Corbi, A. Onori, C. Passananti, A. Romano, Nuclear Factor xB-Dependent Histone Acetylation is Specifically Involved in Persistent Forms of Memory. J. Neurosci. 33, 7603-7614 (2013).
73. R. J. van Oort, M. D. McCauley, S. S. Dixit, L. Pereira, Y. Yang, J. L. Respress, Q. Wang, A. C. De Almeida, D. G. Skapura, M. E. Anderson, D. M. Bers, X. H. T. Wehrens, Ryanodine receptor phosphorylation by calcium/calmodulin-dependent protein kinase II promotes life-threatening ventricular arrhythmias in mice with heart failure. Circulation 122, 2669-2679 (2010).
74. X. Pan, L. Philippen, S. K. Lahiri, C. Lee, S. H. Park, T. A. Word, N. Li, K. E. Jarrett, R. Gupta, J. O. Reynolds, J. Lin, G. Bao, W. R. Lagor, X. H. T. Wehrens, In Vivo Ryr 2 Editing Corrects Catecholaminergic Polymorphic Ventricular Tachycardia. Circ. Res. 123, 953-963 (2018).
75. S.-J. Park, D. Zhang, Y. Qi, Y. Li, K. Y. Lee, V. J. Bezzerides, P. Yang, S. Xia, S. L. Kim, X. Liu, F. Lu, F. S. Pasqualini, P. H. Campbell, J. Geva, A. E. Roberts, A. G. Kleber, D. J. Abrams, W. T. Pu, K. K. Parker, Insights Into the Pathogenesis of Catecholaminergic Polymorphic Ventricular Tachycardia From Engineered Human Heart Tissue. Circulation 140,390-404 (2019).
76. S. Verstovsek, R. A. Mesa, J. Gotlib, V. Gupta, J. F. DiPersio, J. V. Catalano, M. W. N. Deininger, C. B. Miller, R. T. Silver, M. Talpaz, E. F. Winton, J. H. Harvey, M. O. Arcasoy, E. O. Hexner, R. M. Lyons, R. Paquette, A. Raza, M. Jones, D. Kornacki, K. Sun, H. Kantarjian, Long-term treatment with ruxolitinib for patients with myelofibrosis: 5-year update from the randomized, double-blind, placebo-controlled, phase 3 COMFORT-I trial. J. Hematol. Oncol. J Hematol Oncol 10, 55 (2017).
77. J. G. Shi, X. Chen, T. Emm, P. A. Scherle, R. F. McGee, Y. Lo, R. R. Landman, E. G. McKeever, N. G. Punwani, W. V. Williams, S. Yeleswaram, The effect of CYP3A4 inhibition or induction on the pharmacokinetics and pharmacodynamics of orally administered ruxolitinib (INCB018424 phosphate) in healthy volunteers. J. Clin. Pharmacol. 52, 809-818 (2012).
78. J. G. Shi, X. Chen, R. F. McGee, R. R. Landman, T. Emm, Y. Lo, P. A. Scherle, N. G. Punwani, W. V. Williams, S. Yeleswaram, The Pharmacokinetics, Pharmacodynamics, and Safety of Orally Dosed INCB018424 Phosphate in Healthy Volunteers. J. Clin. Pharmacol. 51, 1644-1654 (2011).
79. Y. Ogama, T. Mineyama, A. Yamamoto, M. Woo, N. Shimada, T. Amagasaki, K. Natsume, A randomized dose-escalation study to assess the safety, tolerability, and pharmacokinetics of ruxolitinib (INC424) in healthy Japanese volunteers. Int. J. Hematol. 97, 351-359 (2013).
80. D. Dobrev, M. Aguilar, J. Heijman, J.-B. Guichard, S. Nattel, Postoperative atrial fibrillation: mechanisms, manifestations and management. Nat. Rev. Cardiol. 16,417-436 (2019).
81. J. P. Mathew, M. L. Fontes, I. C. Tudor, J. Ramsay, P. Duke, C. D. Mazer, P. G. Barash, P. H. Hsu, D. T. Mangano, for the Investigators of the Ischemia Research and Education Foundation and the Multicenter Study of Perioperative Ischemia Research Group, A Multicenter Risk Index for Atrial Fibrillation After Cardiac Surgery. JAMA 291, 1720-1729 (2004).
82. R. H. Mehta, A. Z. Starr, R. D. Lopes, J. S. Hochman, P. Widimsky, K. S. Pieper, P. W. Armstrong, C. B. Granger, for the APEX AMI Investigators, Incidence of and Outcomes Associated With Ventricular Tachycardia or Fibrillation in Patients Undergoing Primary Percutaneous Coronary Intervention. JAMA 301, 1779-1789 (2009).
83. J. Heijman, A. P. Muna, T. Veleva, C. E. Molina, H. Sutanto, M. Tekook, Q. Wang, I. H. Abu-Taha, M. Gorka, S. Künzel, A. El-Armouche, H. Reichenspurner, M. Kamler, V. Nikolaev, U. Ravens, N. Li, S. Nattel, X. H. T. Wehrens, D. Dobrev, Atrial Myocyte NLRP3/CaMKII Nexus Forms a Substrate for Postoperative Atrial Fibrillation. Circ. Res. 127, 1036-1055 (2020).
84. J. R. Bell, M. Vila-Petroff, L. M. D. Delbridge, CaMKII-dependent responses to ischemia and reperfusion challenges in the heart. Front. Pharmacol. 5, 96 (2014).
85. Y. Tsuji, M. Hojo, N. Voigt, A. El-Armouche, Y. Inden, T. Murohara, D. Dobrev, S. Nattel, I. Kodama, K. Kamiya, Ca2+-Related Signaling and Protein Phosphorylation Abnormalities Play Central Roles in a New Experimental Model of Electrical Storm. Circulation 123, 2192-2203 (2011).
86. T. M. Roston, K. Jones, N. M. Hawkins, J. M. Bos, P. J. Schwartz, F. Perry, M. J. Ackerman, Z. W. M. Laksman, P. Kaul, K. V. V. Lieve, J. Atallah, A. D. Krahn, S. Sanatani, Implantable cardioverter-defibrillator use in catecholaminergic polymorphic ventricular tachycardia: A systematic review. Heart Rhythm 15, 1791-1799 (2018).
87. S. Mishra, N. Sadagopan, B. Dunkerly-Eyring, S. Rodriguez, D. C. Sarver, R. P. Ceddia, S. A. Murphy, H. Knutsdottir, V. P. Jani, D. Ashok, C. U. Oeing, B. O'Rourke, J. A. Gangoiti, D. D. Sears, G. W. Wong, S. Collins, D. A. Kass, Inhibition of phosphodiesterase type 9 reduces obesity and cardiometabolic syndrome in mice. J. Clin. Invest. 131 (2021), doi:10.1172/JCI148798.
88. N. Pavlaki, K. A. De Jong, B. Geertz, V. O. Nikolaev, A. Froese, Cardiac Hypertrophy Changes Compartmentation of cAMP in Non-Raft Membrane Microdomains. Cells 10, 535 (2021).
89. S. Eid, S. Turk, A. Volkamer, F. Rippmann, S. Fulle, KinMap: a web-based tool for interactive navigation through human kinome data. BMC Bioinformatics 18, 16 (2017).
90. P. C. Taylor, T. Takeuchi, G. R. Burmester, P. Durez, J. S. Smolen, W. Deberdt, M. Issa, J. R. Terres, N. Bello, K. L. Winthrop, Safety of baricitinib for the treatment of rheumatoid arthritis over a median of 4.6 and up to 9.3 years of treatment: final results from long-term extension study and integrated database. Ann. Rheum. Dis. 81, 335-343 (2022).
91. H. S. Rugo, J. Huober, J. A. Garcia-Shenz, N. Masuda, J. H. Sohn, V. A. M. Andre, S. Barriga, J. Cox, M. Goetz, Management of Abemaciclib-Associated Adverse Events in Patients with Hormone Receptor-Positive, Human Epidermal Growth Factor Receptor 2-Negative Advanced Breast Cancer: Safety Analysis of MONARCH 2 and MONARCH 3. The Oncologist 26, e53-e65 (2021).
92. S. Johnston, M. Martin, A. Di Leo, S.-A. Im, A. Awada, T. Forrester, M. Frenzel, M. C. Hardebeck, J. Cox, S. Barriga, M. Toi, H. Iwata, M. P. Goetz, MONARCH 3 final PFS: a randomized study of abemaciclib as initial therapy for advanced breast cancer. Npj Breast Cancer 5, 1-8 (2019).
93. M. J. Borad, L.-Y. Bai, M.-H. Chen, J. M. Hubbard, K. Mody, S. Y. Rha, D. A. Richards, S. L. Davis, J. Soong, C.-E. C.-E. Huang, E. Tse, D. H. Ahn, H.-M. Chang, C.-J. Yen, D.-Y. Oh, J. O. Park, C. Hsu, C. R. Becerra, J.-S. Chen, Y.-Y. Chen, Silmitasertib (CX-4945) in combination with gemcitabine and cisplatin as first-line treatment for patients with locally advanced or metastatic cholangiocarcinoma: A phase Ib/II study. J. Clin. Oncol. 39, 312-312 (2021).
94. E. S. Wang, A. D. Goldberg, R. B. Walter, R. Collins, R. M. Stone, Long-term results of a phase 2 trial of crenolanib combined with 7±3 chemotherapy in adults with newly diagnosed FLT3 mutant AML. J. Clin. Oncol. 40, 7007-7007 (2022).
95. M. Wu, C. Li, X. Zhu, FLT3 inhibitors in acute myeloid leukemia. J. Hematol Oncol. J Hematol Oncol 11, 133 (2018).
96. Incyte Corporation, JAKAFI® (ruxolitinib) tablets, for oral use [package insert] (available at https://www.accessdata.fda.gov/drugsatfda_docs/label/2021/202192 s0231bl.pdf).
97. Eli Lilly and Company, OLUMIANT (baricitinib) tablets, for oral use (available at https://www.accessdata.fda.gov/drugsatfda_docs/label/2022/207924 s0061bl.pdf).
98. S. C. J. Jorgensen, C. L. Y. Tse, L. Burry, L. D. Dresser, Baricitinib: A Review of Pharmacology, Safety, and Emerging Clinical Experience in COVID-19. Pharmacotherapy 40, 843-856 (2020).
99. Eli Lilly and Company, VERZENIO® (abemaciclib) tablets, for oral use (available at https://www.accessdata.fda.gov/drugsatfda_docs/label/2021/208716 s006 s007 s0081bl.pdf). 100. A. Galanis, H. Ma, T. Rajkhowa, A. Ramachandran, D. Small, J. Cortes, M. Levis, Crenolanib is a potent inhibitor of FLT3 with activity against resistance-conferring point mutants. Blood 123, 94-100 (2014).
101. S. R. D. Johnston, et al. Abemaciclib Combined With Endocrine Therapy for the Adjuvant Treatment of HR+, HER2−, Node-Positive, High-Risk, Early Breast Cancer (monarchE). J. Clin. Oncol. 38, 3987-3998 (2020).
102. J. K. Randhawa, H. M. Kantarjian, G. Borthakur, P. A. Thompson, M. Konopleva, N. Daver, N. Pemmaraju, E. Jabbour, T. M. Kadia, Z. Estrov, A. Ramachandran, J. Paradela, M. Andreef, M. Levis, F. Ravandi, J. E. Cortes, Results of a Phase II Study of Crenolanib in Relapsed/Refractory Acute Myeloid Leukemia Patients (Pts) with Activating FLT3 Mutations. Blood 124, 389 (2014).
103. C. Gavegnano, W. B. Haile, S. Hurwitz, S. Tao, Y. Jiang, R. F. Schinazi, W. R. Tyor, Baricitinib reverses HIV-associated neurocognitive disorders in a SCID mouse model and reservoir seeding in vitro. J. Neuroinflammation 16, 182 (2019).
104. T. J. Raub, G. N. Wishart, P. Kulanthaivel, B. A. Staton, R. T. Ajamie, G. A. Sawada, L. M. Gelbert, H. E. Shannon, C. Sanchez-Martinez, A. D. Dios, Brain Exposure of Two Selective Dual CDK4 and CDK6 Inhibitors and the Antitumor Activity of CDK4 and CDK6 Inhibition in Combination with Temozolomide in an Intracranial Glioblastoma Xenograft. Drug Metab. Dispos. 43, 1360-1371 (2015).

### Other Embodiments

From the foregoing description, it will be apparent that variations and modifications may be made to the disclosure described herein to adopt it to various usages and conditions. Such embodiments are also within the scope of the following claims.

All citations to sequences, patents and publications in this specification are herein incorporated by reference to the same extent as if each independent patent and publication was specifically and individually indicated to be incorporated by reference.

1. A synthetic biosensor comprising:
an enzyme substrate;
a detectably labelled protein; and
a phospho-amino acid binding protein.

2. The synthetic biosensor of claim 1, wherein the enzyme substrate is a kinase.

3. The synthetic biosensor of claim 2, wherein the kinase is calcium/calmodulin-dependent protein kinase II (CaMKII).

4. The synthetic biosensor of claim 3, wherein the CaMKII substrate comprises an amino acid sequence having at least about 70% sequence identity to MHRQETVDCLK (SEQ ID NO: 1).

5. The synthetic biosensor of claim 4, wherein the CaMKII substrate comprises an amino acid sequence having at least about 90% sequence identity to MHRQETVDCLK (SEQ ID NO: 1).

6. The synthetic biosensor of claim 4, wherein the CaMKII substrate comprises an amino acid sequence having at least about 95% sequence identity to MHRQETVDCLK (SEQ ID NO: 1).

7. The synthetic biosensor of claim 4, wherein the CaMKII substrate comprises the amino acid sequence MHRQETVDCLK (SEQ ID NO: 1).

8. The synthetic biosensor of claim 1, wherein the detectably labelled protein comprises a radio labeled molecule, a fluorophore, a radiochemical, a luminescent compound, an electron-dense reagent, an enzyme, biotin, a radioactive compound, a non-radioactive compound, digoxigenin or a hapten.

9. (canceled)

10. The synthetic biosensor of claim 1, further comprising an intracellular or extracellular localization sequence.

11. The synthetic biosensor of claim 10, wherein the localization sequence is fused to the biosensor.

12. The synthetic biosensor of claim 10, wherein the localization sequence is covalently linked through a flexible linker.

13. (canceled)

14. An expression vector encoding a biosensor comprising:
an enzyme substrate;
a detectably labelled protein, and a
phospho-amino acid binding protein.

15. The expression vector of claim 14, wherein the enzyme substrate is a kinase.

16. The expression vector of claim 14, wherein the kinase is calcium/calmodulin-dependent protein kinase II (CaMKII).

17. The expression vector of claim 16, wherein the CaMKII substrate comprises an amino acid sequence having at least about 70% sequence identity to MHRQETVDCLK (SEQ ID NO: 1).

18. The expression vector of claim 17, wherein the CaMKII substrate comprises an amino acid sequence having at least about 90% sequence identity to MHRQETVDCLK (SEQ ID NO: 1).

19-21. (canceled)

22. A host cell comprising the synthetic biosensor of claim 1.

23. A method of identifying modulators of kinases comprising:
contacting a biosensor or a cell expressing a biosensor, with one or more candidate agents, wherein the biosensor comprises an enzyme substrate, a detectably labelled protein, and a phospho-amino acid binding protein;
assaying for the presence or absence of a signal from the detectable label; and,
identifying modulators of kinases.

24-53. (canceled)

54. A kit comprising the synthetic biosensor of claim 1.

55. A method of preventing and treating a heart disease comprising administering to a subject in need thereof, a pharmaceutical composition comprising a therapeutically effective amount of ruxolitinib, crenolanib, baricitinib, abemaciclib, silmitasertib, or combinations thereof.

56-61. (canceled)


SEQUENCE LISTING (2 sequences)

SEQ ID NO: 1 - protein, 11 aa, Homo sapiens
MHRQETVDCLK

SEQ ID NO: 2 - protein, 21 aa, synthetic construct
  VARIANT 1: note = amino acids analogs or variants thereof
  VARIANT 2: note = amino acids analogs or variants thereof
  VARIANT 3: note = amino acids analogs or variants thereof
  VARIANT 4: note = amino acids analogs or variants thereof
  VARIANT 5: note = amino acids analogs or variants thereof
  VARIANT 17: note = amino acids analogs or variants thereof
  VARIANT 18: note = amino acids analogs or variants thereof
  VARIANT 19: note = amino acids analogs or variants thereof
  VARIANT 20: note = amino acids analogs or variants thereof
  VARIANT 21: note = amino acids analogs or variants thereof
  VARIANT 1..5: note = This region may encompass 1-5 residues
  VARIANT 17..21: note = This region may encompass 1-5 residues
XXXXXMHRQETVDCLKXXXXX